(19) United States
(12) Patent Application Publication
(10) Pub. No.: US 2025/0115681 A1
(43) Pub. Date: Apr. 10, 2025

(54) CONJUGATES OF NUCLEIC ACIDS OR DERIVATIVES THEREOF AND CELLS, METHODS OF PREPARATION, AND USES THEREOF

(71) Applicant: WESTLAKE UNIVERSITY, Hangzhou, Zhejiang (CN)

(72) Inventors: Zhike LU, Hangzhou, Zhejiang (CN); Lijia MA, Hangzhou, Zhejiang (CN); Yingzheng LIU, Hangzhou, Zhejiang (CN)

(21) Appl. No.: 18/833,709

(22) PCT Filed: Jan. 20, 2023
(86) PCT No.: PCT/CN2023/073366

§ 371 Date: Jul. 26, 2024

(30) Foreign Application Priority Data
Jan. 28, 2022 (WO) PCT/CN2022/074563

(51) Int. Cl. C07K 19/00; C12N 9/52; C12Q 1/6806

(52) U.S. Cl. CPC C07K 19/00; C12N 9/52; C12Q 1/6806; C12Y 304/2207; C12Y 304/22071

(57) ABSTRACT

The present disclosure provides a conjugate of a nucleic acid or derivative thereof and a sortase. The present disclosure also provides a conjugate of a nucleic acid or derivative thereof and a cell, and a method of preparing such a conjugate mediated by a sortase. The present disclosure further provides a method of delivering a nucleic acid or derivative thereof to a cell, mediated by a sortase.

### CROSS-REFERENCE TO RELATED APPLICATION

This application claims priority to International Patent Application No. PCT/CN2022/074563, filed on Jan. 28, 2022, the contents of which are hereby incorporated by reference.

### SEQUENCE LISTING

This application contains a Sequence Listing as an XML file entitled “Seq.xml” having a size of 86 KB and created on Jan. 20, 2023. The information contained in the Sequence Listing is incorporated by reference herein.

### FIELD

The present disclosure relates to a novel reaction of a nucleic acid or derivative mediated by a sortase, as well as products of such a reaction, and uses of such a reaction and such products.

### BACKGROUND

Sortase (Srt), e.g., Sortase A (SrtA) Sortase B (SrtB), sortase C (SrtC), sortase D (SrtD), sortase E (SrtE), and sortase F (SrtF), is a group of transpeptidases that mediate attaching peptides to bacteria cell walls and assembling pili 1. In Staphylococcus aureus (S. aureus), peptides with an LPXTG motif were reported to be recognized by SrtA and were covalently anchored to an NH₂-GGG peptide on the cell wall through a transpeptidation reaction, in which the LPXTG motif served as a sorting signal (the first substrate) and the NH₂-GGG served as a nucleophile² (the second substrate).

Although sortase, such as Sortase A, is not essential for bacterial viability, it attracts broad interests as it displays a diverse array of proteins to bacterial surface²². These displayed surface proteins immediately interact with bacterial environment and participating in essential bacterial physiological and pathological processes, e.g., formation of biofilm and mediating host cell entry^{2, 3}. Thus, sortase is recognized as an import virulence factor and conserved in gram positive bacteria.

So far, natural substrates that can be recognized by a sortase as sorting signals are amino acid motifs, although the sequence of the amino acid motifs are distinct across different sortase classes^{1, 2}. As for the nucleophile, an N-terminal penta-glycine is known to be the canonical substrate of SrtA. However, other nucleophiles, including amino sugar ⁴ (e.g., puromycin) and an internal lysine side chain can also serve as nucleophiles through isopeptide bonds ⁵. Molecules with unbranched primary amines can serve as nucleophiles to ligate with an LPXTG-containing moiety as well ⁶.

To extend the applications of Srt-mediated bioconjugation, diverse enzyme variants of Srt with distinct characteristics have been developed. Liu and colleagues employed yeast display to evolve a sortase variant (eSrtA, 5M), with a 140-fold increase in recognizing activity to an LPETG (SEQ ID NO: 68)-containing peptide ⁷. Another variant, Srt7M, has been later demonstrated that it can mediate bioconjugation between an LPXTG substrate and various amines⁶. Chen and colleagues developed a FRET-based platform for directed evolution, and identified another variant (Chen2016) based on the 5M variant, which significantly improved kinetics of the conjugation reaction ⁸. Chen and colleagues later evolved a “promiscuous” SrtA variant, mgSrtA, which is capable of attaching an LPXTG-containing peptide to N-terminal monoglycine instead of oligoglycine ⁹. Recently, an LMVGG (SEQ ID NO: 69)-recognizing SrtA variant was developed, which enabled sortagging (sortase-mediated transpeptidation) of an endogenous amyloid-β (Aβ) protein, an Alzheimer's disease (AD)-associated protein m. With these efforts, sortagging now can enable efficient bioconjugates of proteins.

### SUMMARY

The inventors surprisingly found that a nucleic acid, e.g., DNA and RNA, or a nucleic acid derivative, e.g., PNA (peptide nucleic acid), can serve as a substrate for a sortase. For example, the inventors surprisingly found that a nucleic acid or a nucleic acid derivative, e.g., DNA oligo, RNA oligo, or PNA, can stably anchor to the surface of a cell in the presence of a sortase, such as mgSrtA. It is unexpected and surprising because sortase has been considered as a transpeptidase that ligates a peptide having a motif such as LPXTG to the N-terminal oligoglycine residues of a protein. Nucleic acids, such as DNA or RNA oligos, have not been reported as substrates for a sortase before. Such a reaction of a nucleic acid or its derivative facilitated by a sortase was previously unknown.

In one embodiment, the present disclosure provides a conjugate of a nucleic acid or derivative thereof and a sortase.

In one embodiment, the present disclosure provides a conjugate of a cell and a nucleic acid or derivative thereof via a sortase.

In one embodiment, the present disclosure provides a nucleic acid comprising an anchor region, preferably guanine enriched, suitable for ligating to a cell. In one embodiment, the present disclosure provides a nucleic acid comprising an anchor region, a region for PCR amplification, a programmable region to distinguish individual cells (e.g., a barcode region), and a capture sequence for sequence enrichment. For example, the anchor region can be enriched with guanine. For another example, the region for PCR amplification can be guanine-depleted. For another example, the capture sequence can be a poly A sequence or a capture sequence suitable for high throughput sequencing.

In one embodiment, the present disclosure provides a method of preparing a conjugate of a cell and a nucleic acid or derivative thereof, comprising contacting the nucleic acid or derivative thereof, the cell, and a sortase, wherein the nucleic acid or derivative thereof is conjugated to the cell, and wherein the conjugation of the nucleic acid or derivative thereof and the cell is mediated by the sortase.

In one embodiment, the present disclosure provides a method of delivering a nucleic acid or derivative thereof to a cell, comprising providing the nucleic acid or derivative thereof and a sortase to the vicinity of the cell, wherein the nucleic acid or derivative thereof is conjugated to the cell mediated by the sortase and wherein the nucleic acid or derivative thereof is internalized into the cell.

In one embodiment, the present disclosure provides a method of identifying a cell, comprising contacting a nucleic acid or derivative thereof, the cell, and a sortase, wherein the nucleic acid or derivative thereof is conjugated to the cell, wherein the conjugation of the nucleic acid or derivative thereof and the cell is mediated by the sortase, and wherein the nucleic acid or derivative thereof comprises an anchor region, a region for PCR amplification, a barcode region, and a capture sequence for sequence enrichment.

In one embodiment, the present disclosure provides a kit comprising a sortase and a nucleic acid or derivative thereof as described herein.

### BRIEF DESCRIPTION OF THE DRAWINGS

FIG. 1 shows a schematic of a method of using a sortase to enhance the efficiency of oligonucleotide drugs by local injection to targeting cells. The top panel (FIG. 1A) illustrates diffusions of the oligonucleotides after local injection without a sortase. The bottom panel (FIG. 1B) illustrates that after local injection with a sortase, the oligonucleotides are conjugated to the cell membranes facilitated by the sortase, which lead to subsequent internalization of the oligonucleotides into the cells.

FIG. 2 shows a schematic of examples of locations for local injections of nucleic acid drugs. The nucleic acid drugs or their bioconjugates can be locally injected with a sortase to (A) tumor sites; (B) epidural sites; (C) intravitreal sites; or (D) intracerebral sites.

FIG. 3 shows a schematic of nucleic acid drugs, delivered to cells as described herein, sensed by receptors in the cells. The receptors may include Toll-like receptors (TLR) on the membrane of endosome, cGAS proteins in cytoplasm, and RIG-I proteins in cytoplasm. The schema shows examples of interactions in the endosome between the heterodimer of TLR7/TLR8 receptors and a single-stranded RNA (ssRNA), between the TLR9 dimer and unmethylated CpG, as well as between the TLR3 dimer and double-stranded RNA (dsRNA). FIG. 3 also shows examples of interactions in the cytoplasm between the cGAS dimer and dsDNA, and between RIG-1 and double-stranded RNA (dsRNA).

FIG. 4 shows a schematic of examples of downstream mechanisms of action by nucleic acid drugs delivered to cells as described herein. FIG. 4A, FIG. 4B, and FIG. 4C illustrate that the nucleic acid drugs can hybridize with a targeting mRNA, resulting in degradation of the mRNA. FIG. 4D and FIG. 4E illustrate that the nucleic acid drugs can serve as steric-blocking oligonucleotides to regulate the expression of a targeting mRNA without degradation of the mRNA. FIG. 4F illustrates that the nucleic acid drugs can also target circular RNA by sequence hybridization and cause degradation of the circular RNA. “RISC” means “RNA-induced silencing complex,” “ASO” means “antisense oligonucleotide,” “mRNA” means “messenger RNA”.

FIG. 5 shows a schematic of protein, peptide, or antigen products produced from nucleic acid drugs delivered into cells, facilitated by a sortase, as described herein. After internalization of the nucleic acid drugs, the nucleic acids are translated in the cytoplasm and their products can go to various intracellular or extracellular destinations for downstream functions. Examples of the destinations include (1) nucleus; (2) cytoplasm; (3) cell membrane; and (4) presentation to extracellular sites by MHC complexes.

FIG. 6A shows fluorescence signals of FITC (Fluorescein isothiocyanate), Biotin (Biotin subsequently detected by Streptavidin-Phycoerythrin, SAv-PE), and TAMRA-modified oligos attached to K562 cells with the presence of mgSrtA. The fluorescence signals of FITC, PE (Biotin), and TAMRA were collected by flow cytometry, and were each plotted across five samples, including a negative control (NC), 4-nt polyadenosine modified respectively by FITC, Biotin, and TAMRA (4-nt polyA), 4-nt polythymine modified respectively by FITC, Biotin, and TAMRA (4-nt polyT), 4-nt polycytosine modified respectively by FITC, Biotin, and TAMRA (4-nt polyC), and 4-nt polyguanine modified respectively by FITC, Biotin, and TAMRA (4-nt polyG).

FIG. 6B shows FITC signals collected from FITC-modified oligonucleotides attached to K562 cells, and plotted across six samples including a negative control (NC), FITC-modified 32-nt polyA (32-nt polyA), FITC-modified 32-nt polyT (32-nt polyT), FITC-modified 32-nt polyC (32-nt polyC), FITC-modified 4-nt polyG (32-nt polyG), and FITC-modified 34-nt mixed nucleotides (34-nt Mix). The sequence of the 34-nt Mix is set forth in SEQ ID NO: 1: GTGACTGGAGTTCAGACGTGTGCTCTTCCGATCT. An amino acid sequence of mgSrtA is set forth in SEQ ID NO: 2:

KPHIDNYLHDKDKDEKIEQYDKNVKEQASKDKKQQAKPQIPKDKSKVAGY
IEIPDADIKEPVYPGPATREQLNRGVSFAEENESLDDQNISIAGHTFIGR
PNYQFTNLKAAKKGSMVYFKVGNETRKYKMTSIRNVKPTAVGVLDEQKGK
DKQLTLITCDDLNRETGVWETRKILVATEVK.

The mgSrtA as used in this application is SEQ ID NO: 2 unless otherwise indicated.

FIG. 7 shows plots of the percentage of the cells positively labeled by FITC, TAMRA and Biotin-modified oligonucleotides and the mean fluorescence intensity of the labeled cells. The biotin quantity was represented by SAv-PE. The cells were labeled with FITC, TAMRA-modified, and Biotin-modified 4-nt or 32-nt polyA, polyT, polyC, or polyG, respectively, with (mgSrtA+) or without (mgSrtA−) the presence of mgSrtA. A FITC-modified 34-nt oligo with mixed A, T, C, and G nucleotides (34Mix) was included to compare the labeling efficiencies of that oligonucleotide (SEQ ID NO: 1). For each 4-nt oligo, three different modifications (FITC, TAMRA, and Biotin) were included to confirm the mgSrtA-dependent labeling of the cells.

FIG. 7A shows fluorescence signals of FITC represented as the percentage of positively labeled cells and the mean fluorescence intensity, respectively, for cells labeled by FITC-modified 4-nt polyA, polyT, polyC, or polyG, respectively, with or without the presence of mgSrtA.

FIG. 7B shows fluorescence signals of TAMRA represented as the percentage of positively labeled cells and the mean fluorescence intensity, respectively, for cells labeled by TAMRA-modified 4-nt polyA, polyT, polyC, or polyG, respectively, with or without the presence of mgSrtA.

FIG. 7C shows fluorescence signals of anti-biotin antibody represented as the percentage of positively labeled cells and the mean fluorescence intensity, respectively, for cells labeled by Biotin-modified 4-nt polyA, polyT, polyC, or polyG, respectively, with or without the presence of mgSrtA.

FIG. 7D shows fluorescence signals of FITC represented as the percentage of positively labeled cells and the mean fluorescence intensity, respectively, for cells labeled by FITC-modified 32-nt polyA, polyT, polyC, or polyG, respectively, with or without the presence of mgSrtA. A FITC-modified 34-nt oligo with mixed A, T, C, and G nucleotides (34Mix) was included to compare the labeling efficiencies of the oligos.

FIG. 8 is a schematic for screening preferred oligonucleotides for cell labeling facilitated by a sortase such as mgSrtA. For example, oligonucleotides of 79-nt were designed, which included a PCR handle, 12-nt random nucleotides, and a polyA tail. The oligonucleotides were incubated with cells at the presence of mgSrtA. The cells labeled with the oligonucleotides were then subjected to a SMART-seq protocol. The oligonucleotides were amplified in two sequential PCR. The first PCR enriched the oligonucleotides from the endogenous RNAs. And the second PCR added the P5 and P7 adapter sequences for high throughput sequencing on an Illumina platform. The screen experiment used an oligonucleotide library (mixed sequences) to label cells rather than an individual oligo with a fixed sequence. The 12-nt random sequence can be referenced as a 12-nt barcode, which is composed of 4¹² possible sequences. At the end of the screen, oligos that labeled the most cells are reflected by the highest abundance from the high throughput sequencing data.

FIG. 9 shows motifs identified from high throughput sequencing after a screen experiment illustrated in FIG. 8. The top panel shows that the guanine nucleotide was dominantly enriched from the screen experiment with the presence of mgSrtA (mgSrtA+). The bottom panel shows the motif analysis without the presence of mgSrtA (mgSrtA−), which served as control. The top and bottom panels of FIG. 9 show the nucleotide distributions across the 12-nt barcode region. The x-axis represented the sequence positions on the 12-nt barcode region, and the y-axis was proportionally occupied by the four different nucleotides. A bigger letter (e.g., “G” at position 1) means a higher proportion of that nucleotide in that position, and a smaller letter (e.g., “T” at position 6) means a lower proportion of that nucleotide in that position.

FIG. 10 shows Cy5 signals collected from cells labeled by Cy5-modified RNA oligos. FIG. 10A shows the mean fluorescence intensity (the left y-axis, also referred to as “MFI”) and the percentage of positively labeled cells (the right y-axis) of both K562 cells and Jurkat cells. The experiments were performed in triplicates. The K562 and Jurkat cells were labeled with RNA oligos of different concentrations, including 50 nM, 100 nM, 500 nM, and 1 μM. “NC” represented blank cells (without mgSrtA or RNA oligo). FIG. 10B shows multi-histograms of the Cy5 fluorescence signals from one representative replicate of the triplicates noted for FIG. 10A. The sequence of the RNA oligo is set forth in SEQ ID NO: 3: G*G*G*GUGGGGCGGGGAAACACAUCCACUACCAACACUCUGCUUUAAGG*C*C*G, in which the “*” means phosphorothioate modification.

FIG. 11 shows FITC fluorescence signals collected from DNA sequences in various strand formats. FIG. 11A shows the FITC signals collected from three replicates. FIG. 11B shows multi-histograms of the FITC fluorescence signals from one representative replicate of the triplicates noted for FIG. 11A. For each format, the strand with a circled “F” represented a 45-nt DNA oligo modified with FITC (denoted as “45*”). In the formats denoted as “45*+30RC” and “45*+45RC,” the bottom strand represented a DNA oligo that was complementary with the 45* strand (the complementary strand of 30-nt or 45-nt denoted as “30RC” or “45RC”). In the formats denoted as “45*+30” and “45*+45,” the bottom strand represented a DNA oligo that shared the same sequence as the 45*, except that the bottom strand (denoted as “30” or “45”) did not have an FITC modification. In each format of “45*+30RC,” “45*+30,” “45*+45RC,” and “45*+45”, the same molar of FITC-modified oligo was mixed with the other oligo. In the formats denoted as 45* and 45, single strand DNA oligos, with an FITC modification or without, were used without the presence of other DNA oligos.

The sequence of the “45*” and “45” is set forth in SEQ ID NO: 4:

ATCGATCGATGCTAGCTAGCGTTCAGACGTGTGCTCTTCCGATCT;

The sequence of the “30RC” is set forth in SEQ ID NO: 5:

ACGTCTGAACGCTAGCTAGCATCGATCGAT;

The sequence of the “30” is set forth in SEQ ID NO: 6:

ATCGATCGATGCTAGCTAGCGTTCAGACGT;

The sequence of the “45RC” is set forth in SEQ ID NO: 7:

AGATCGGAAGAGCACACGTCTGAACGCTAGCTAGCATCGATCGAT.

FIG. 12 shows FITC signals collected from cell labeling using DNA sequences in various strand formats. The “Cell only” column represented blank cells without mgSrtA or single-stranded or double-stranded DNA sequences; and the other columns represented cells labeled by DNA oligos in presence of mgSrtA. ss*: a 20-nt (dark bar) or 60-nt (grey bar) FITC modified DNA oligo; ss*+ss: two 20-nt (dark bar) or two 60-nt (grey bar) DNA oligos having the same sequence but only one of two 20-nt oligos or only one of two 60-nt oligos was FITC-modified; ss*+ss(RC): a 20-bp (dark bar) or 60-bp (grey bar) double-stranded DNA with one strand modified by FITC.

The sequence of the “ss*” or “ss” of 20-nt is set forth in SEQ ID NO: 8:

ATCGATCGATGCTAGCTAGC;

The sequence of the “ss(RC)” of 20-nt is set forth in SEQ ID NO 9:

GCTAGCTAGCATCGATCGAT;

The sequence of the “ss*” or “ss” of 60-nt is set forth in SEQ ID NO 10:

ATCGATCGATGCTAGCTAGCGTTCAGACGTGTGCTCTTCCGATCTGTGAC
TGGAGTTCAG;

The sequence of the “ss(RC)” of 60-nt is set forth in SEQ ID NO 11:

CTGAACTCCAGTCACAGATCGGAAGAGCACACGTCTGAACGCTAGCTAGC
ATCGATCGAT.

FIG. 13 shows Phycoerythrin (PE) signals collected from cells labeled by biotin-modified PNA (peptide nucleic acids). The PE signals quantitatively represented the biotin through the affinity between the biotin and a streptavidin-PE antibody. FIG. 13A shows that cells were labeled by PNA in the presence of mgSrtA. “Cell only” means blank cells pre-stained with streptavidin-PE antibodies as the other samples. FIG. 13B shows the multi-histogram showing the fluorescence signals from one representative replicate out of the triplicate experiments in FIG. 13A. FIG. 13C shows the structure of the PNA.

FIG. 14A shows confocal images showing the distribution of TAMRA signals in K562 cells labeled by TAMRA-modified DNA oligo. FIG. 14B shows confocal images showing the distribution of FITC signals in K562 cells labeled by FITC-modified DNA oligo. FIG. 14C shows confocal images showing the distribution of Cy5 signals in K562 cells labeled by Cy5-modified DNA oligo. From top to bottom in FIGS. 14A, 14B, and 14C, each row represented a sample with (“+”) or without (denoted as “-”) the presence of mgSrtA or oligo. TD: transmitted light detector. Oligonucleotide (+) concentration: 100 nM; mgSrtA(+) concentration: 20 uM. “Merge” means a confocal image wherein the fluorescence image (TAMRA, FITC, or Cy5) and the image captured under transmitted light (TD) were merged.

The sequence of the 3′-TAMRA-modified DNA oligo is set forth in SEQ ID NO: 12:

GGGGCGGGGTGGGGCGGGGAAATCATCTCAACCACTCACATCCACTACC
AACACTCTHHACTCACTTAAHHHHHBAAAAAAAAAAAAAAAAAAAAAAA
AA;

The sequence of the 3′-FITC-modified DNA oligo is set forth in SEQ ID NO: 13:

GGGGCGGGGTGGGGGGGGGAAATCATCTCAACCACTCACATCCACTACC
AACACTCTHHAACATATCTCHHHHHBAAAAAAAAAAAAAAAAAAAAAAA
AA;

The sequence of the 3′-Cy5-modified DNA oligo is set forth in SEQ ID NO: 14:

GGGGCGGGGTGGGGCGGGGAAATCATCTCAACCACTCACATCCACTACC
AACACTCTHHAACATATCTCHHHHHBAAAAAAAAAAAAAAAAAAAAAAA
AA.

In the sequences of SEQ ID NO: 12, 13 and 14, the letter H represented A, C or T nucleotide and the letter B represented C, G, or T nucleotide.

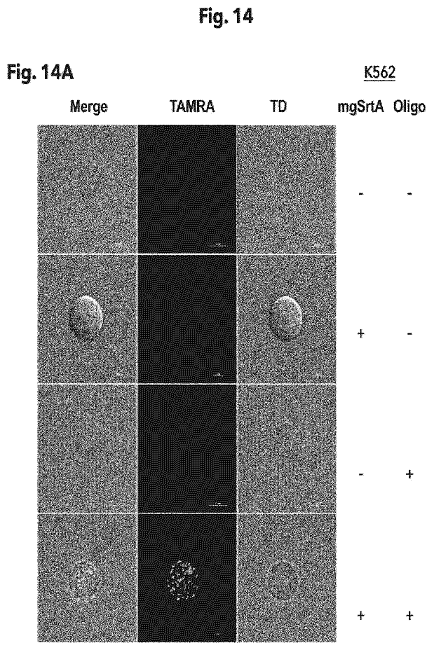

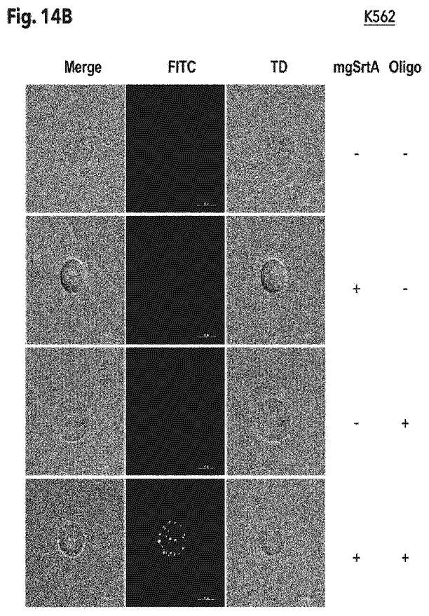

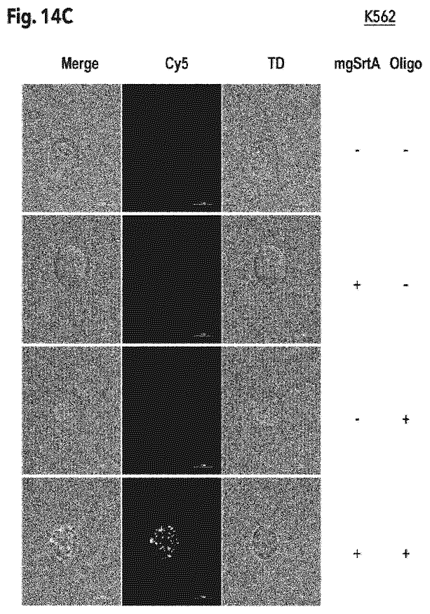

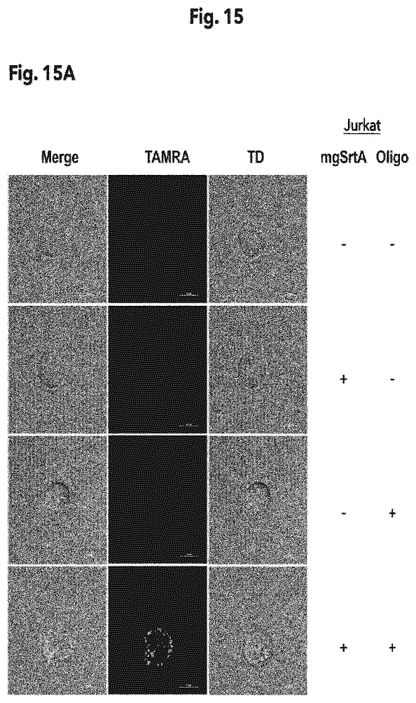

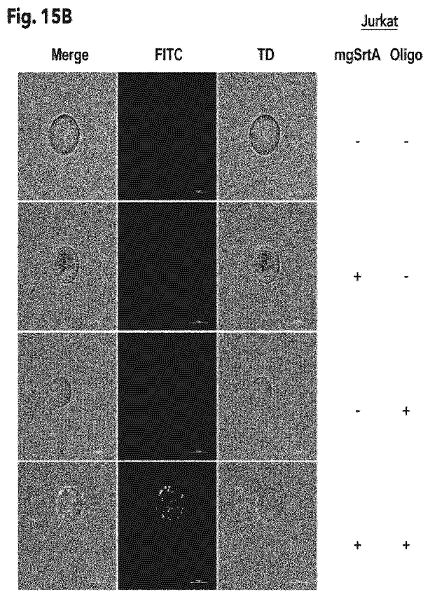

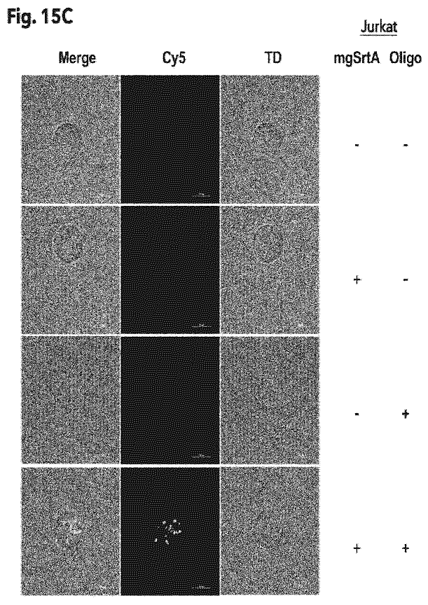

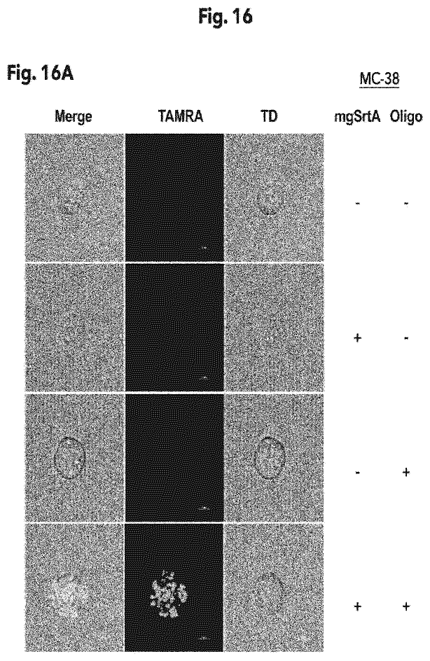

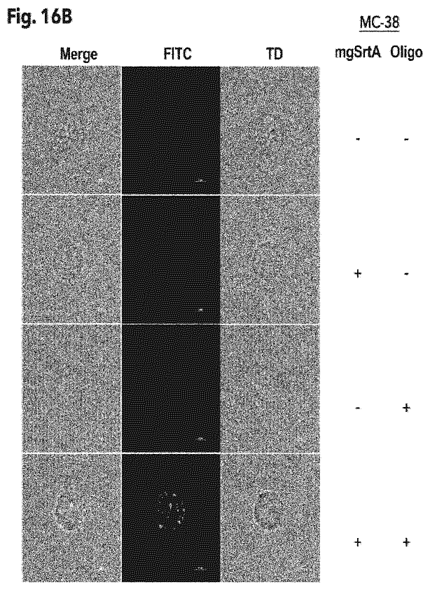

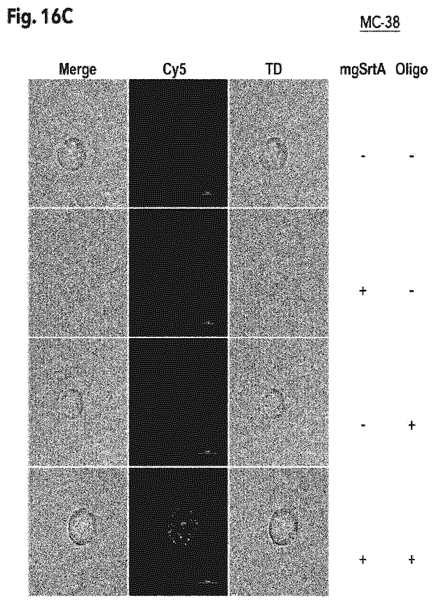

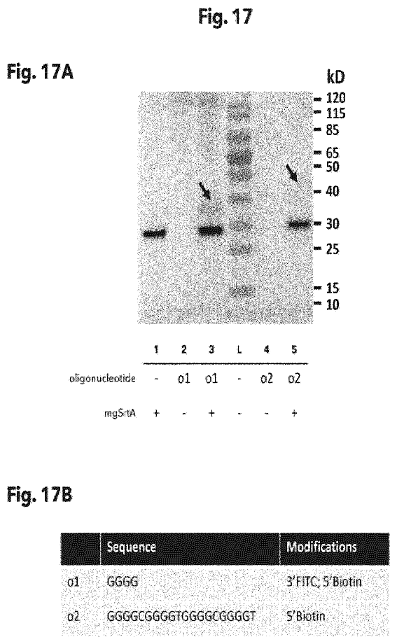

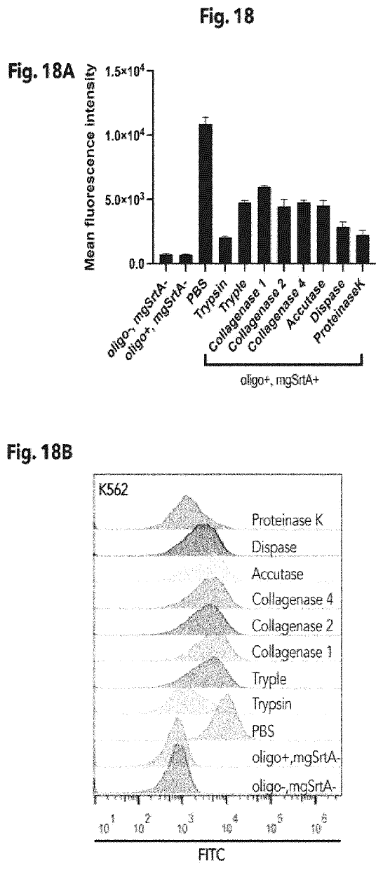

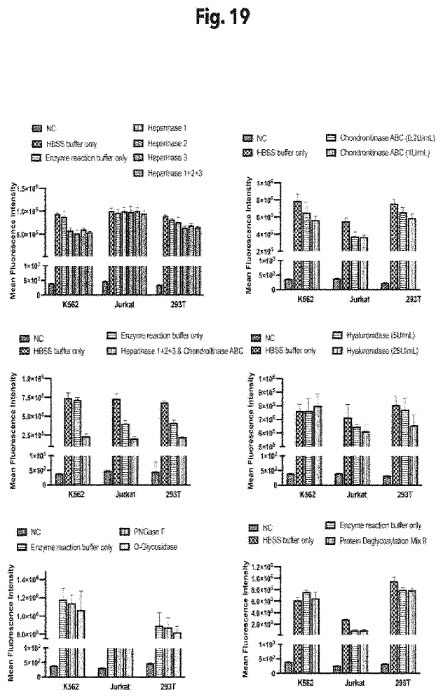

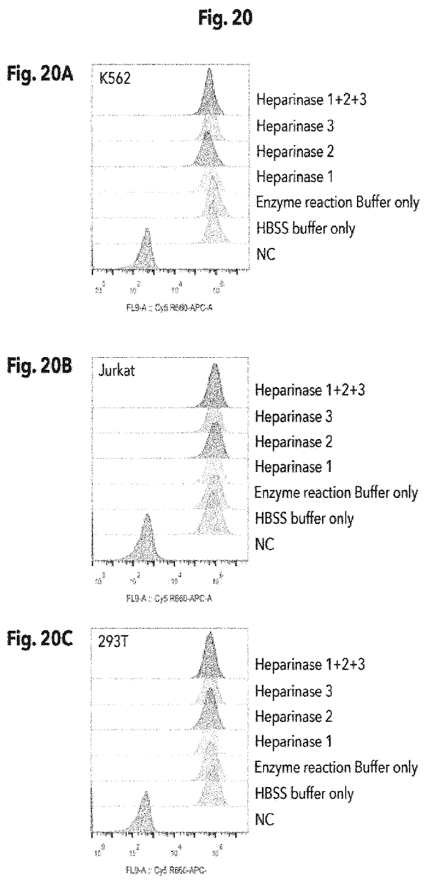

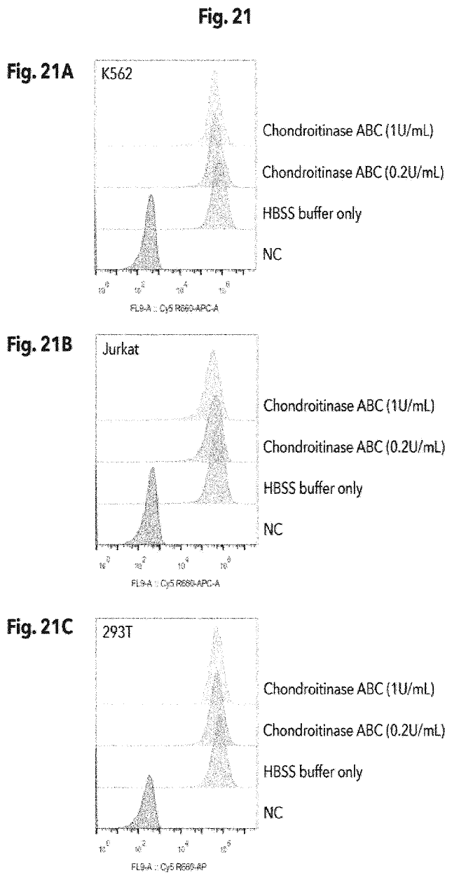

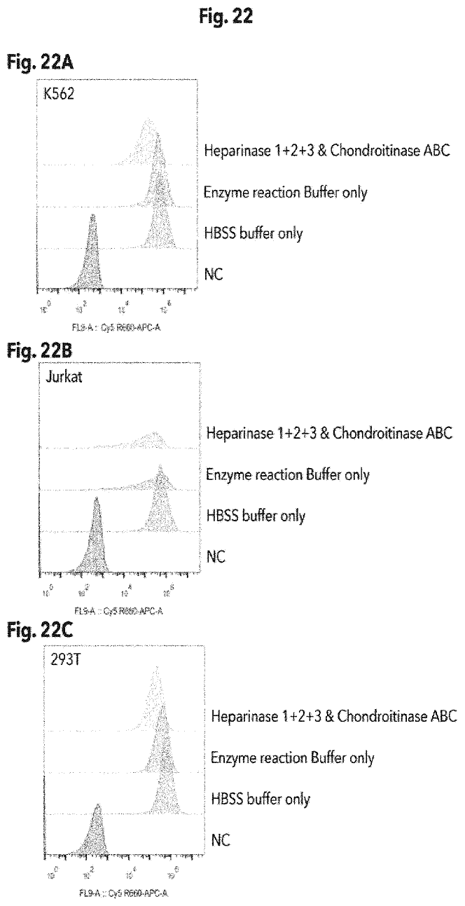

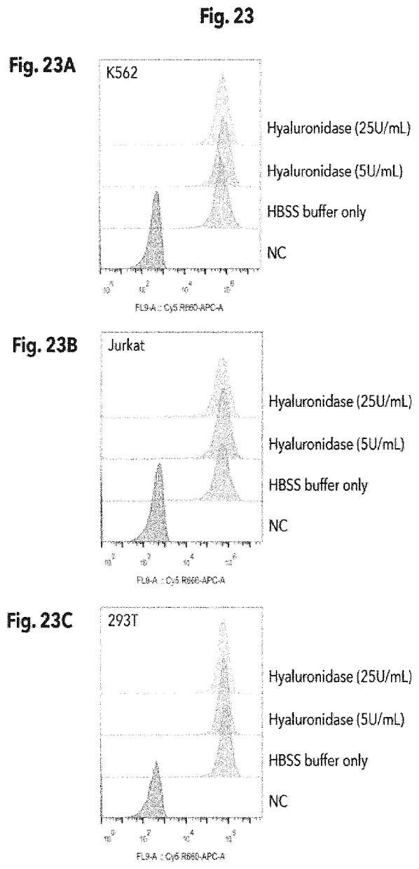

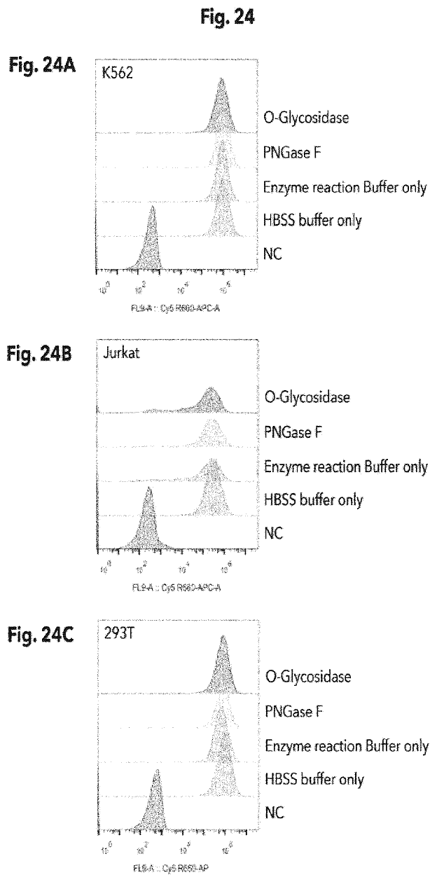

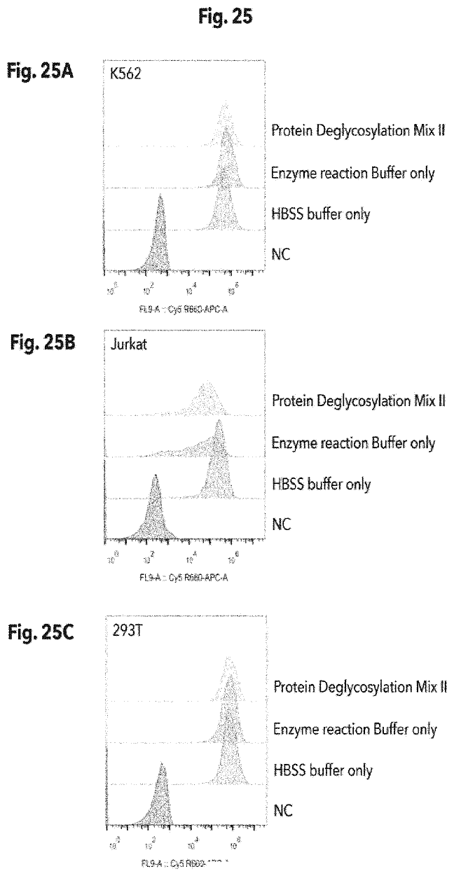

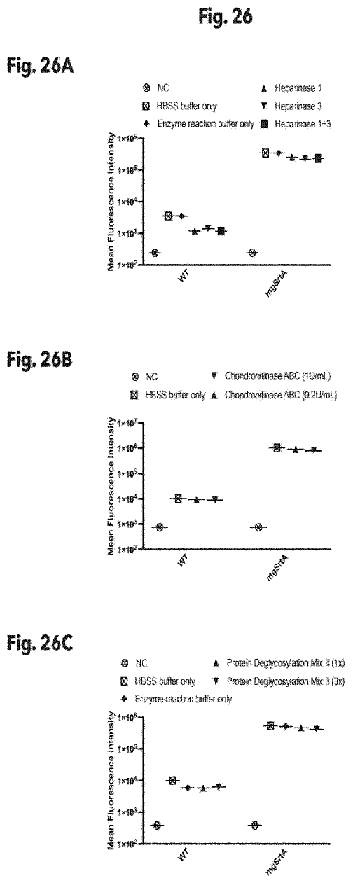

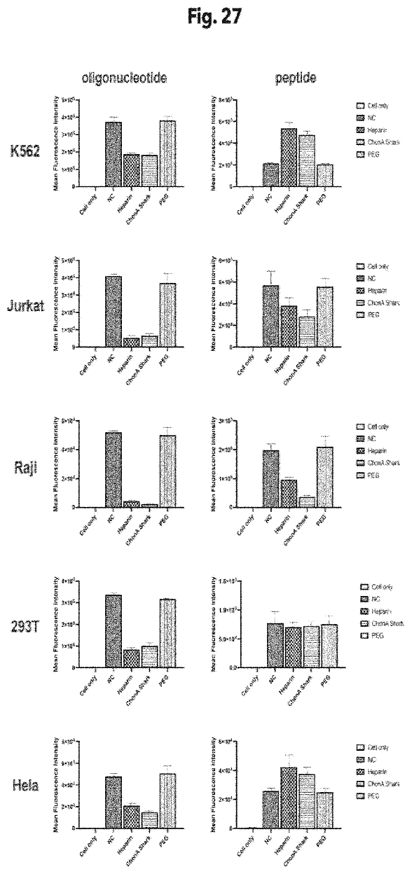

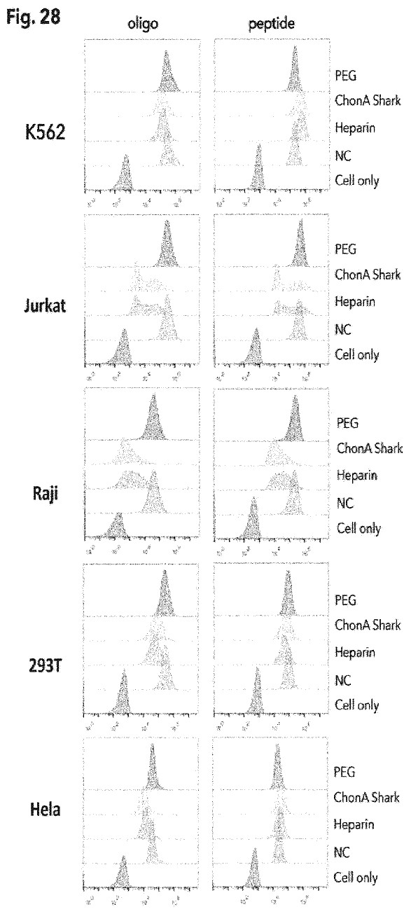

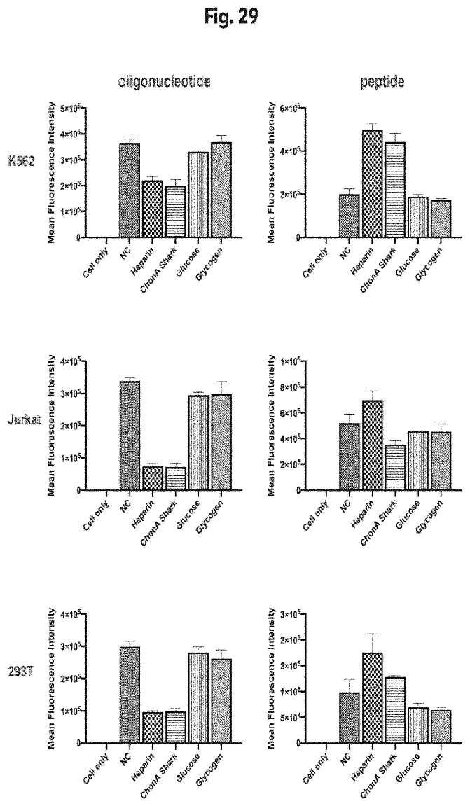

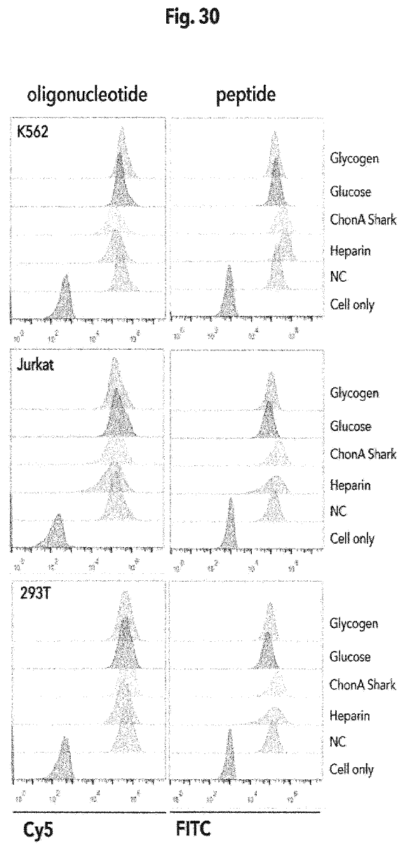

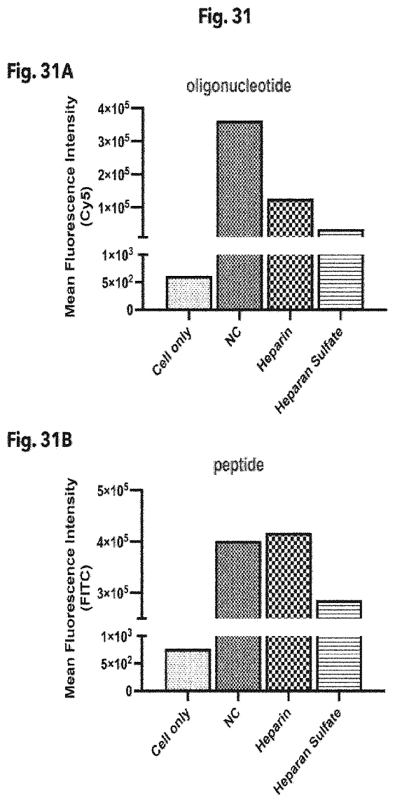

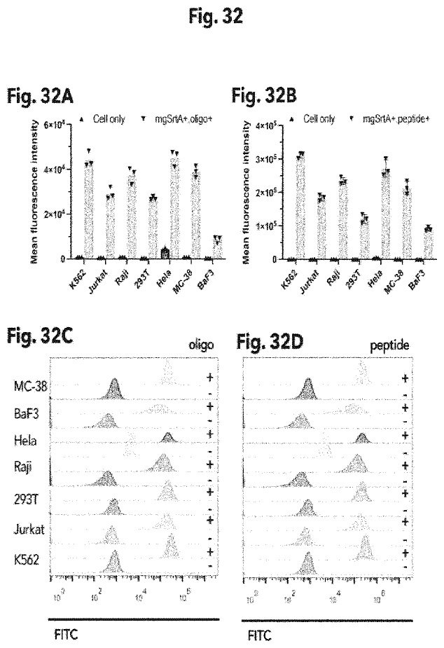

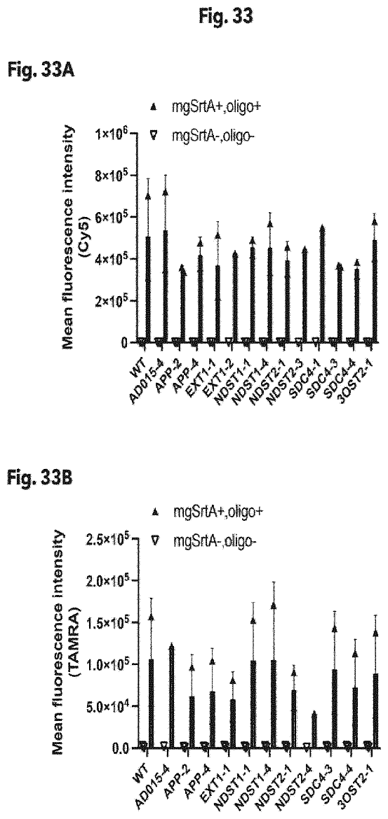

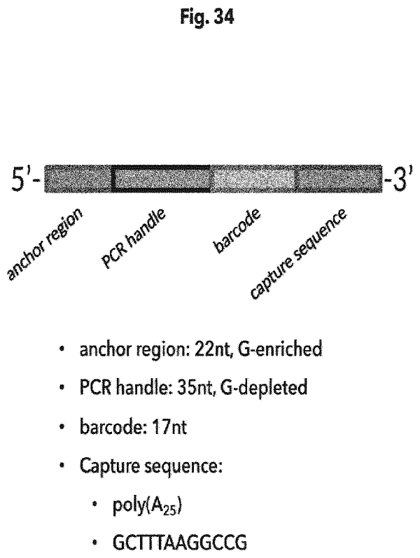

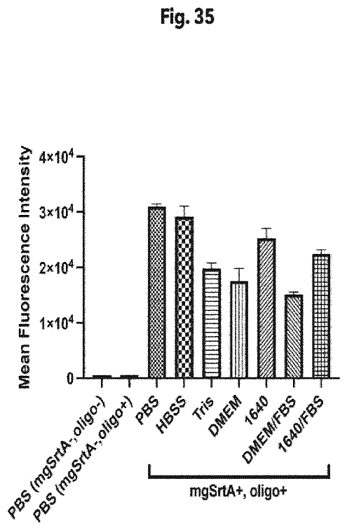

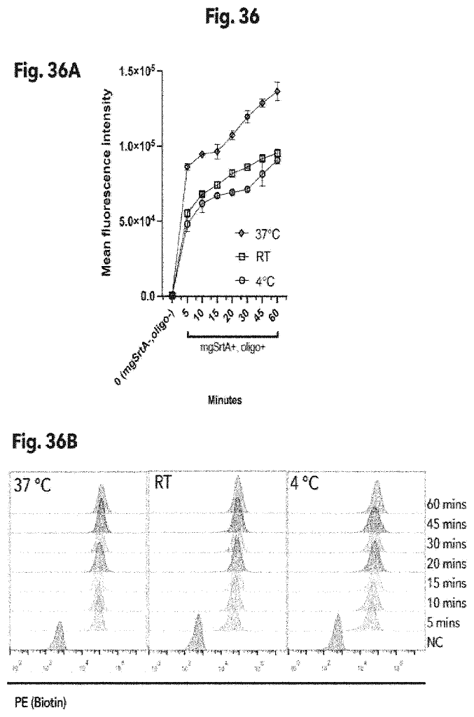

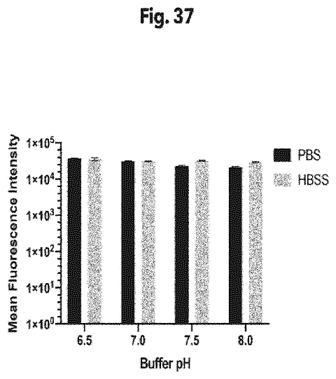

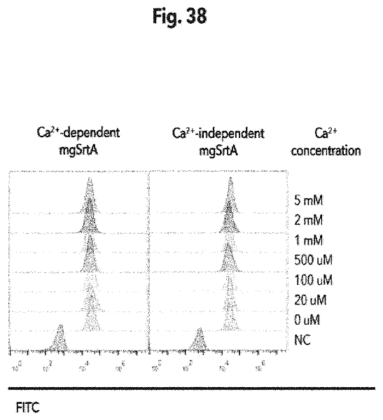

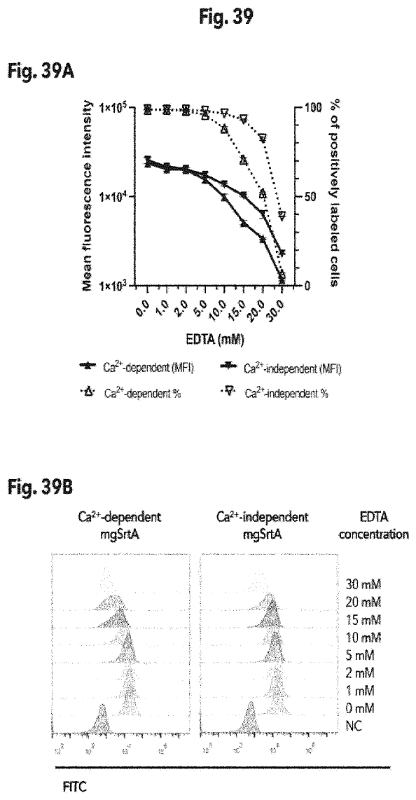

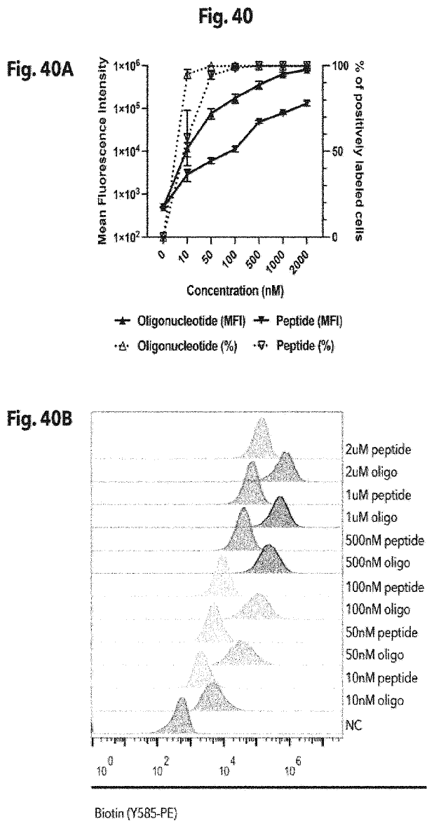

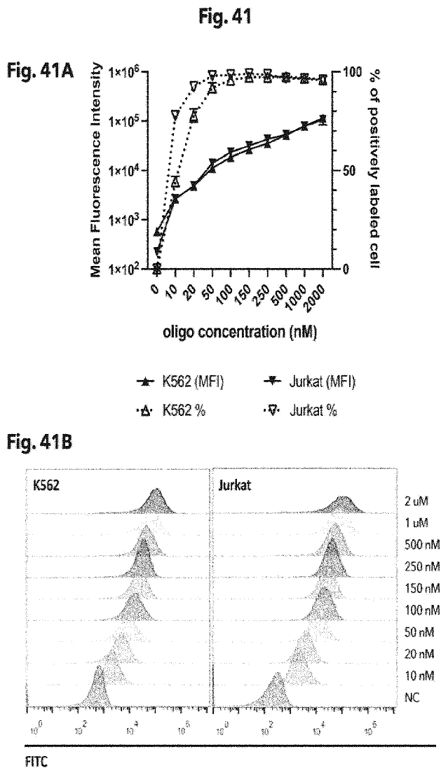

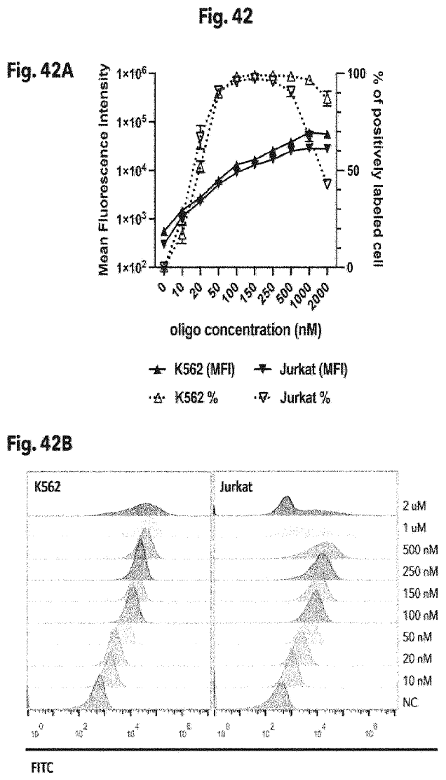

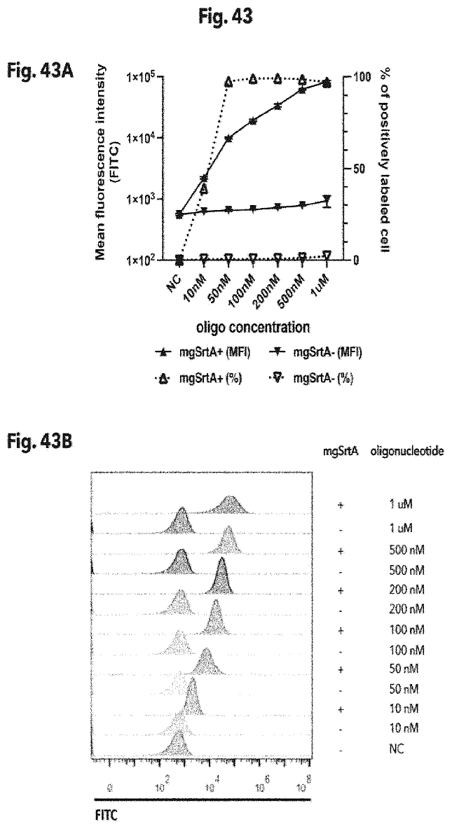

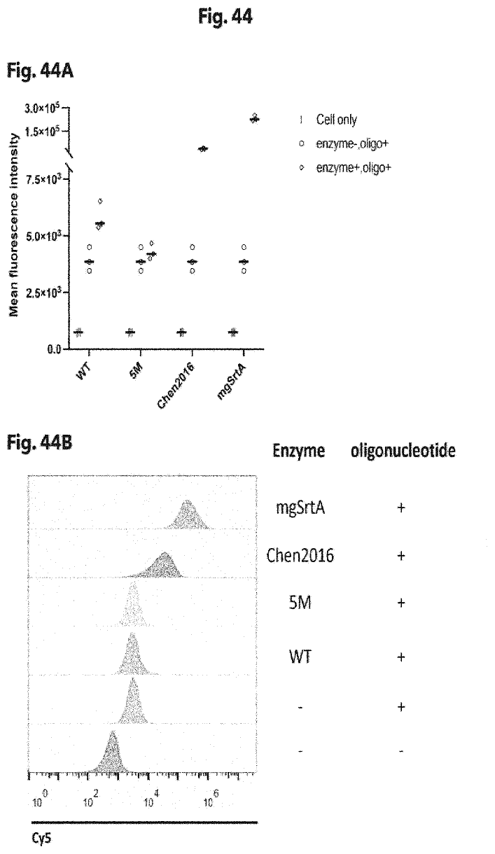

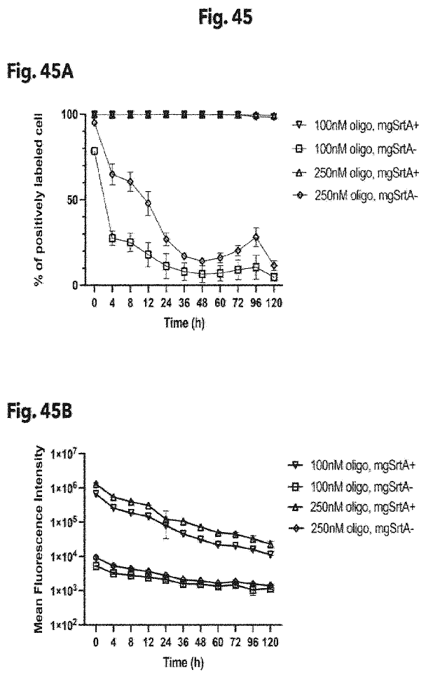

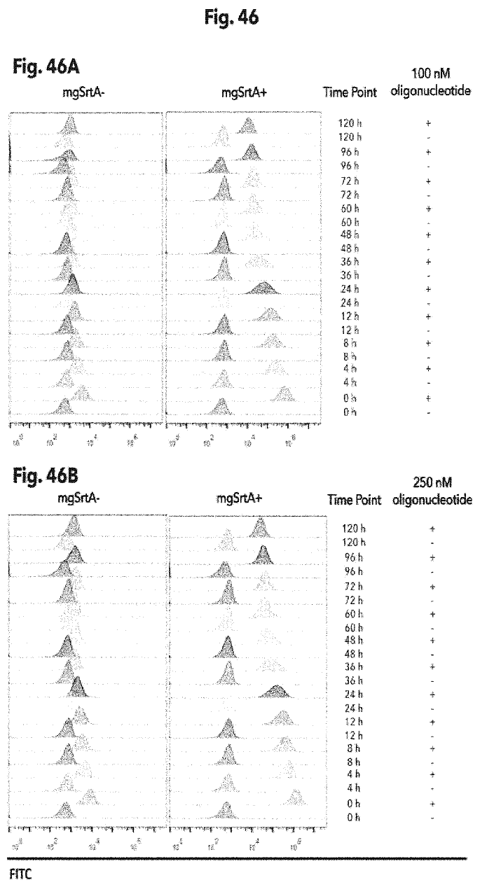

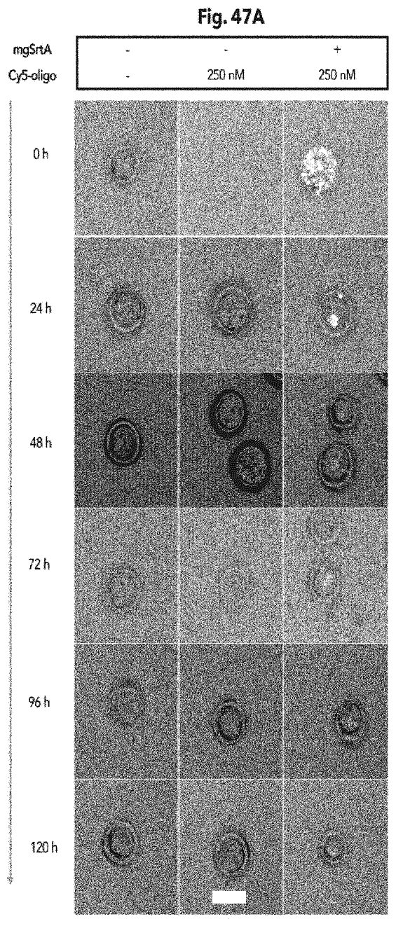

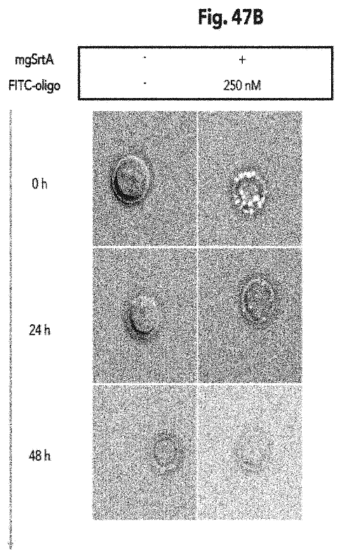

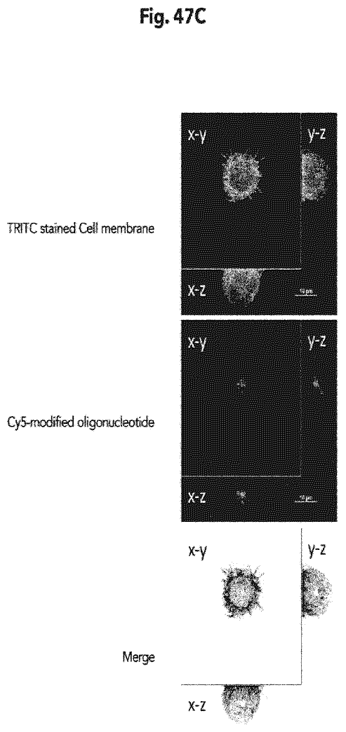

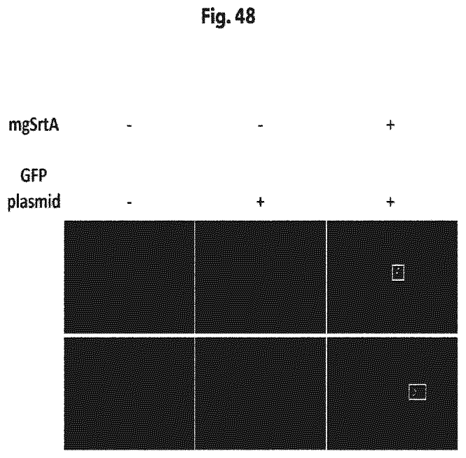

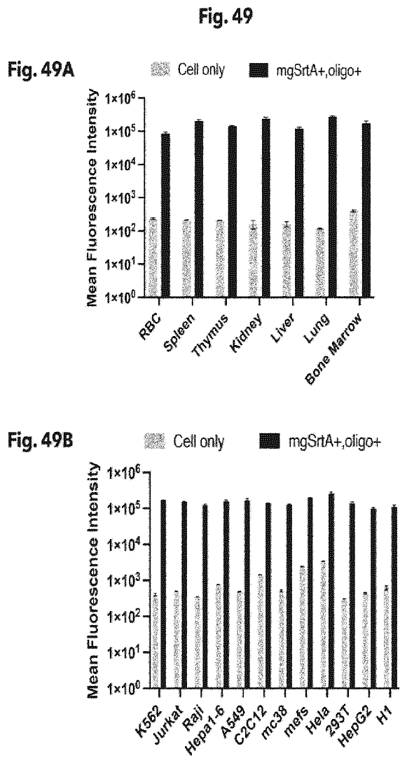

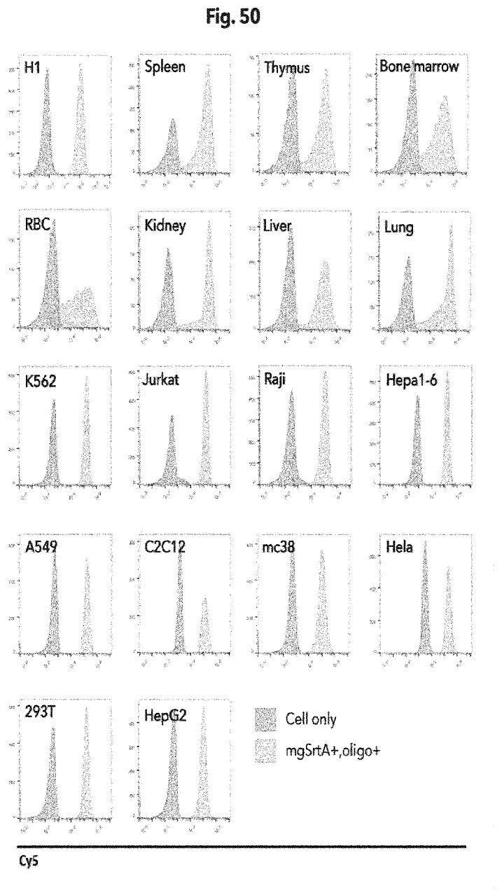

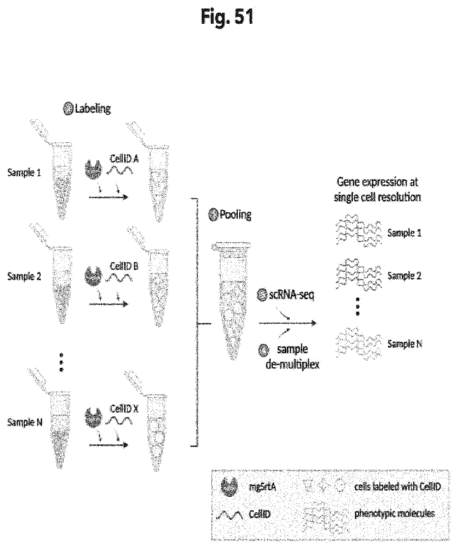

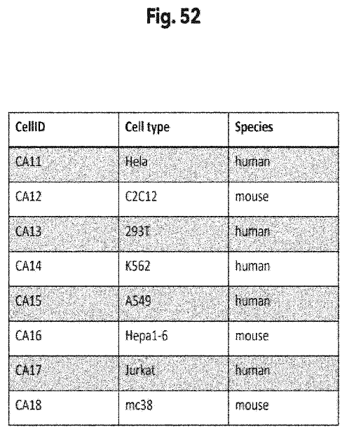

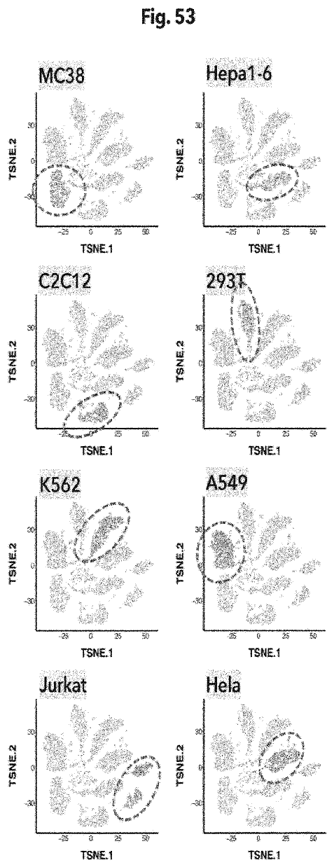

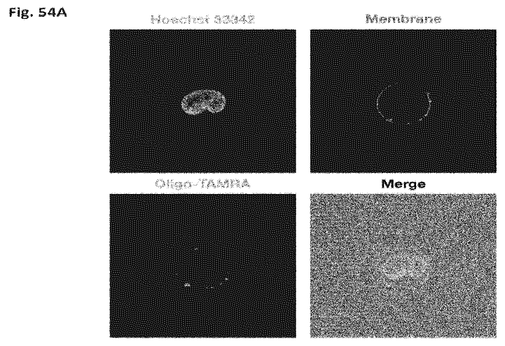

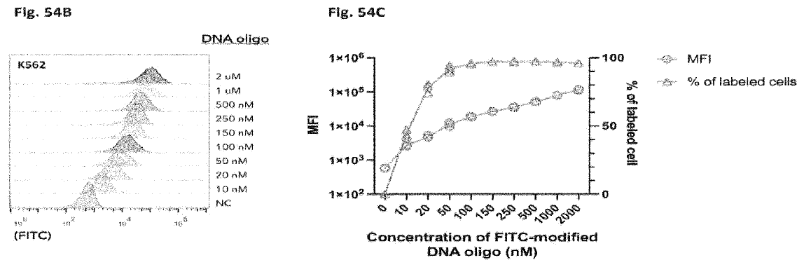

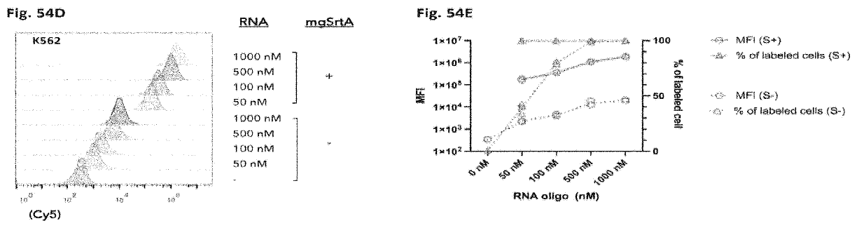

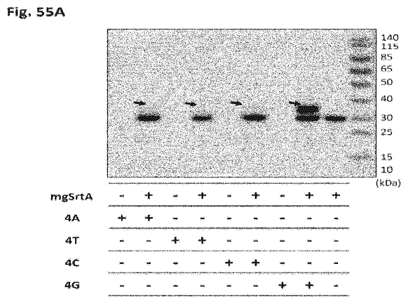

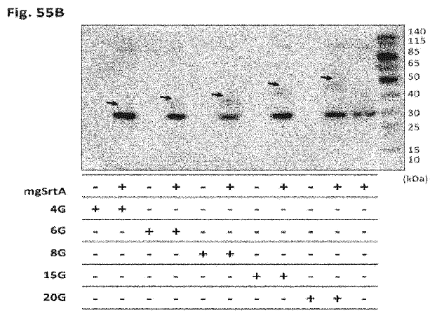

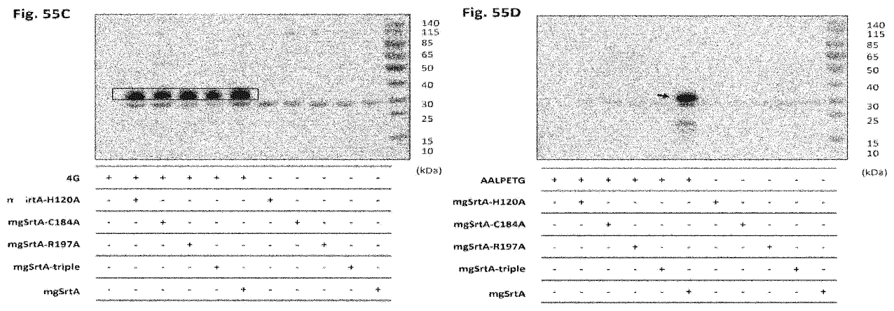

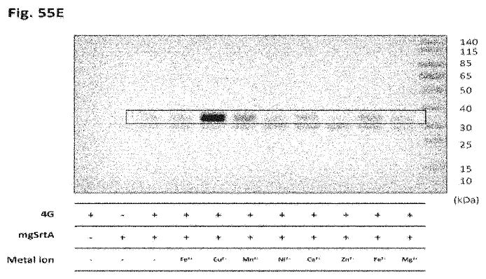

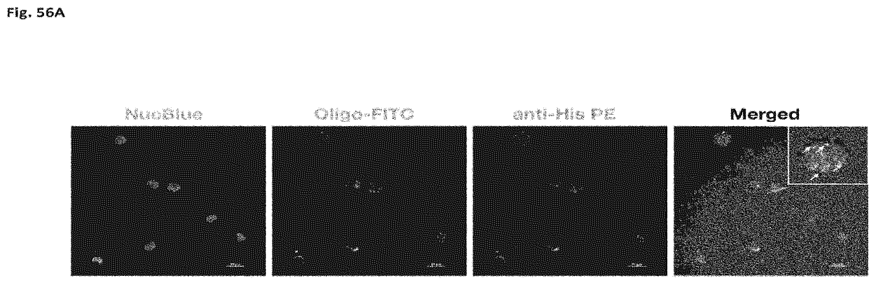

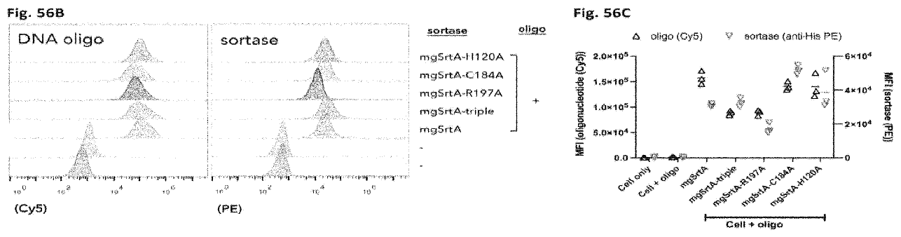

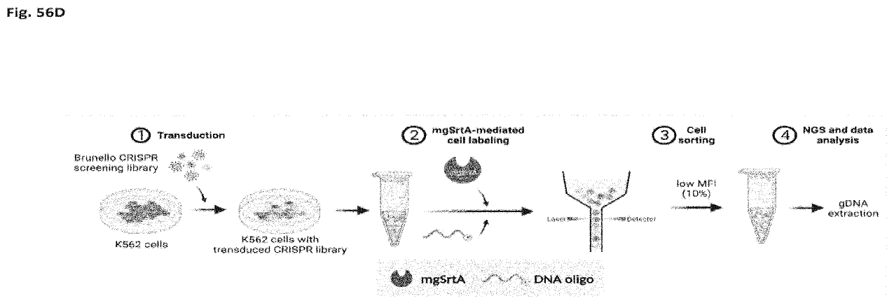

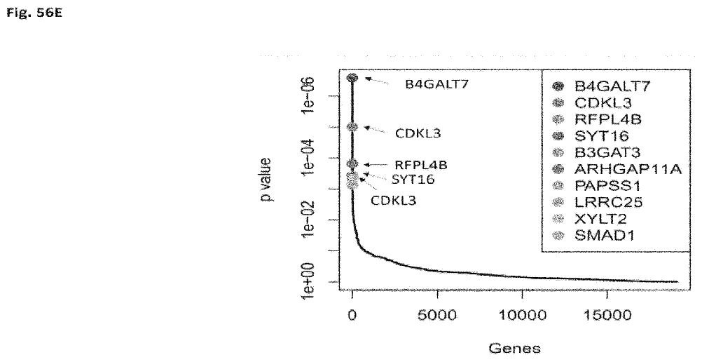

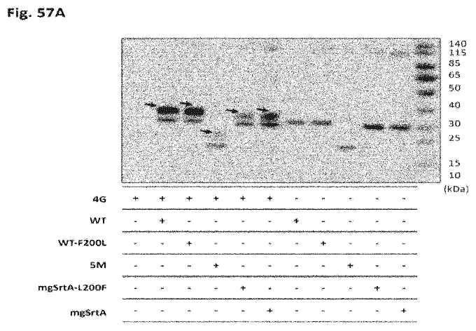

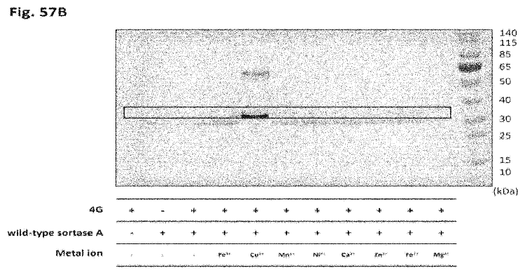

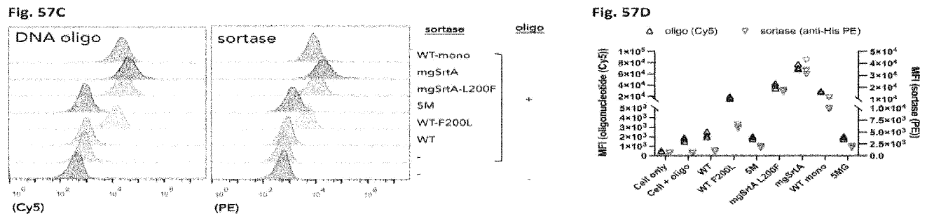

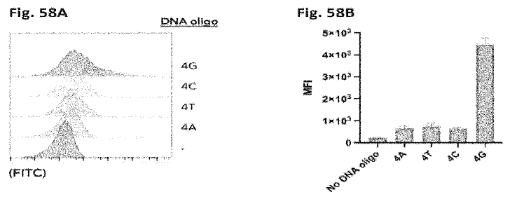

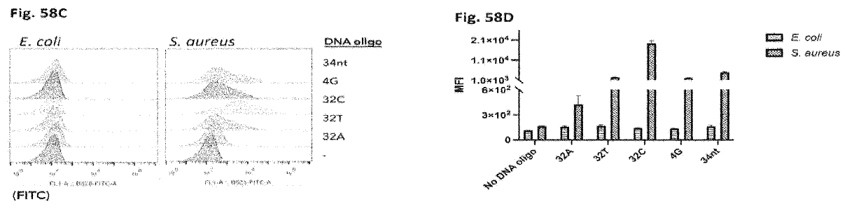

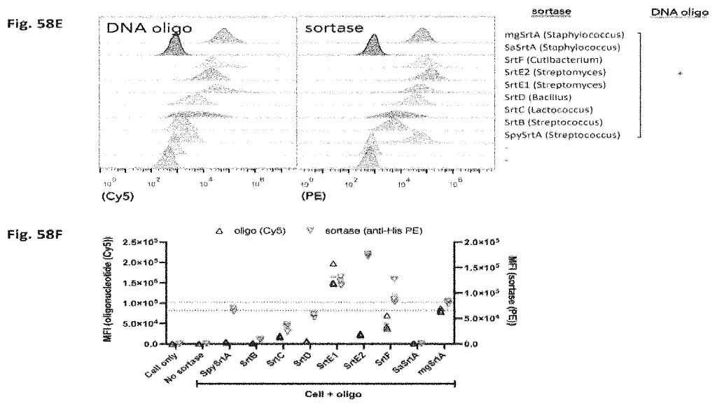

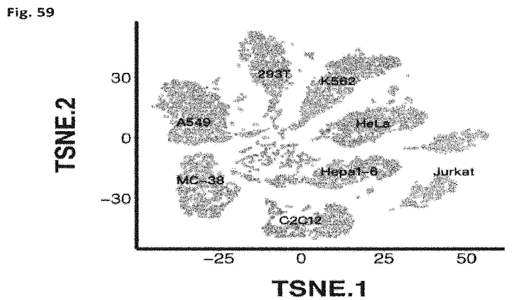

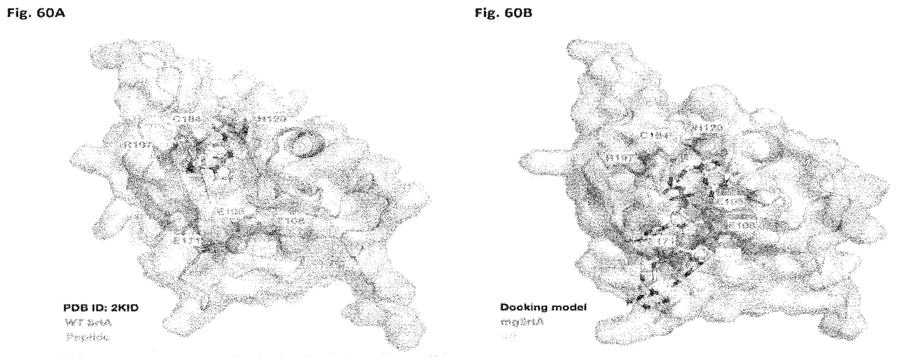

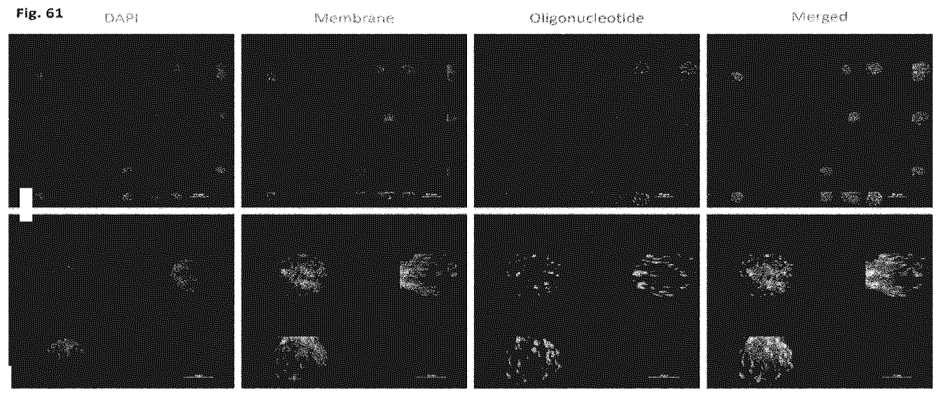

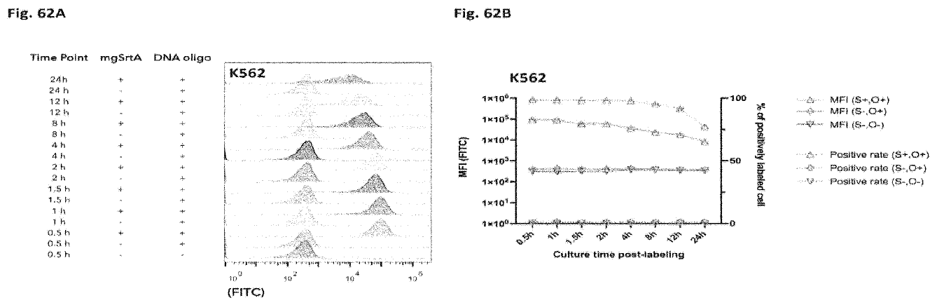

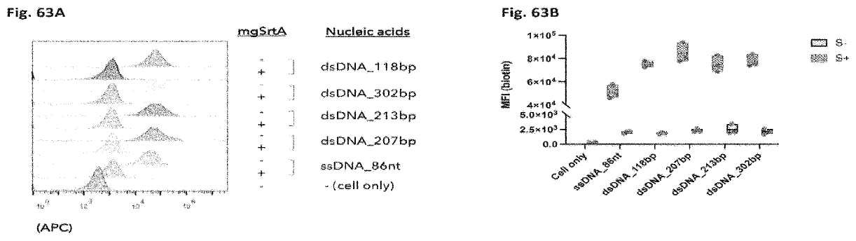

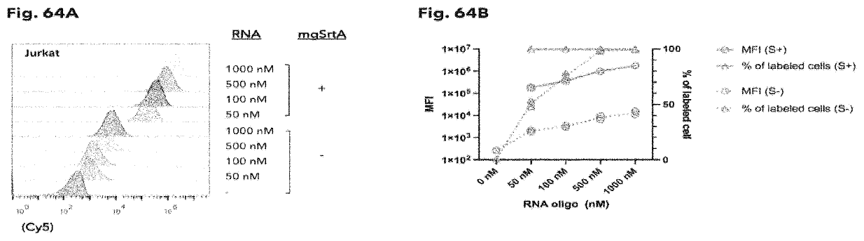

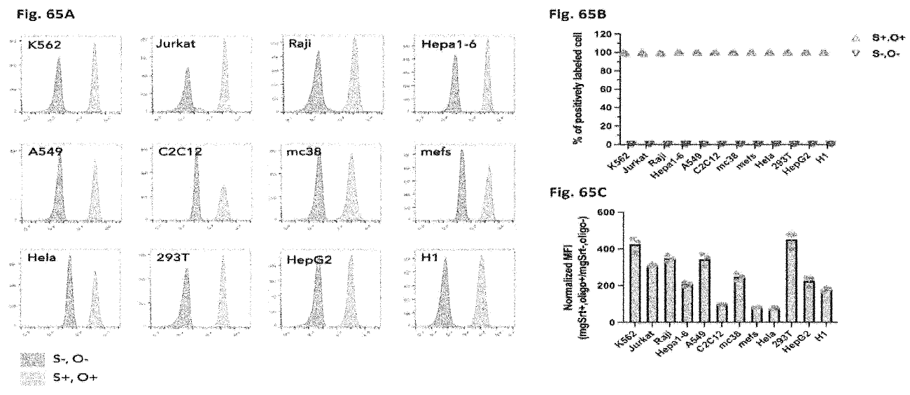

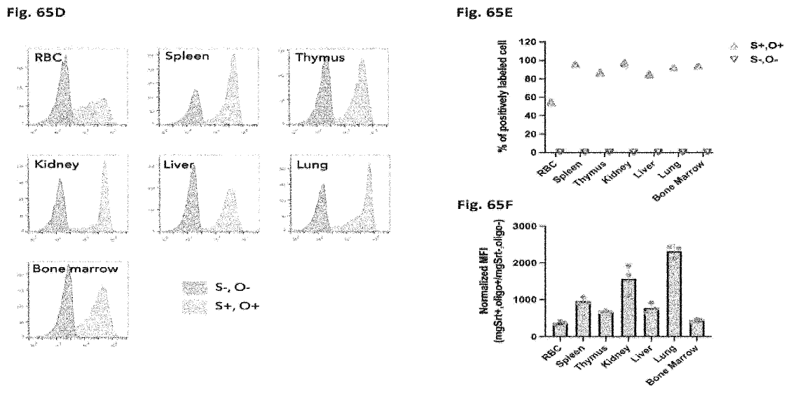

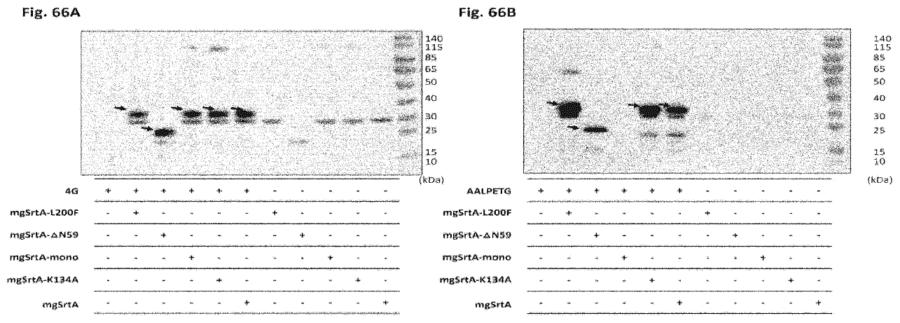

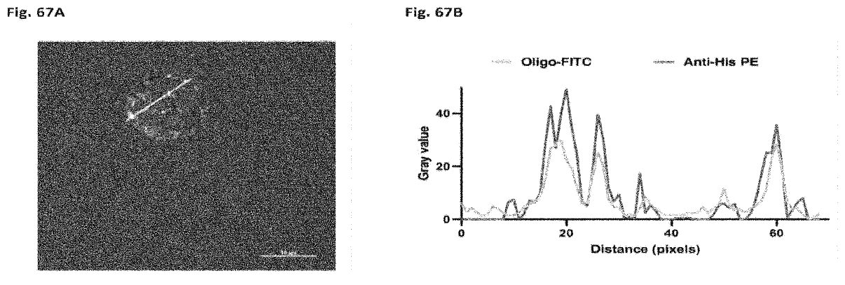

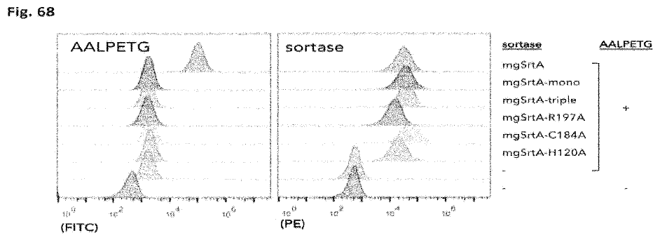

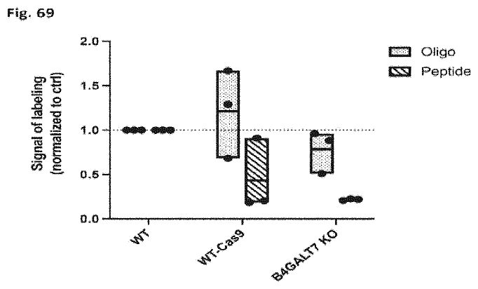

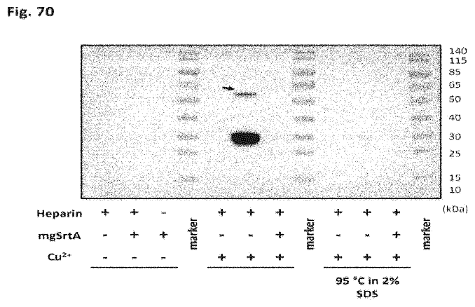

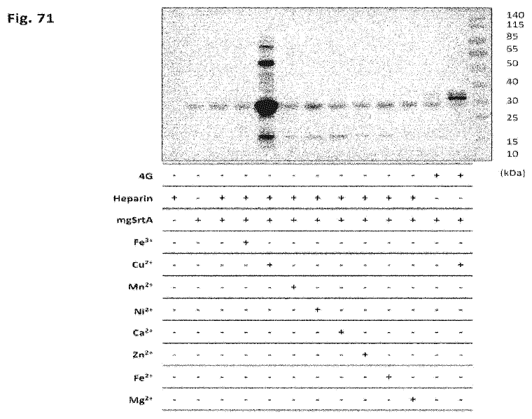

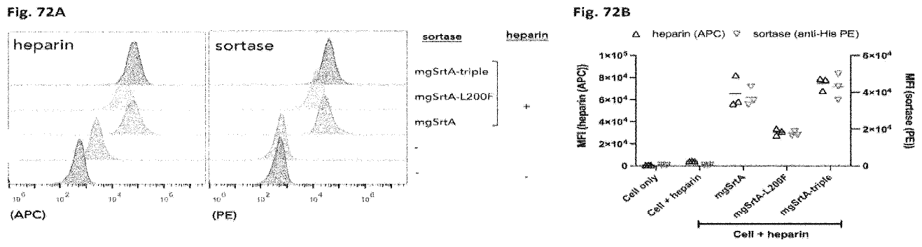

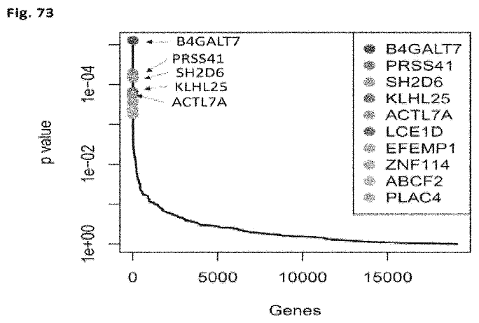

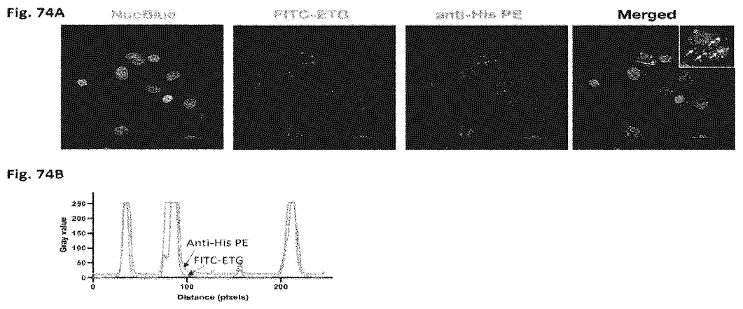

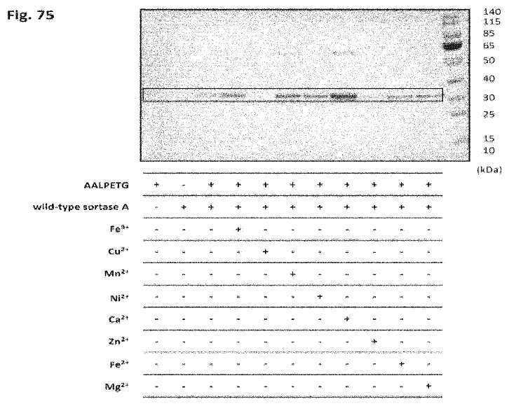

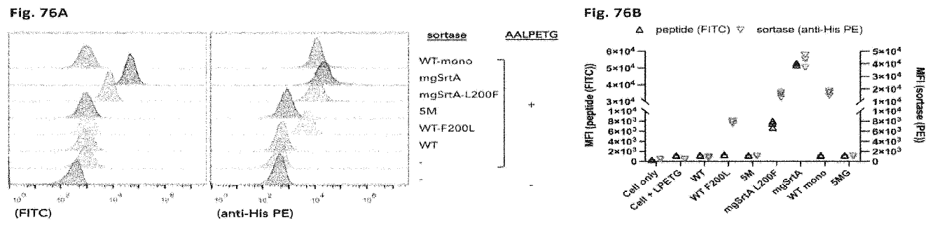

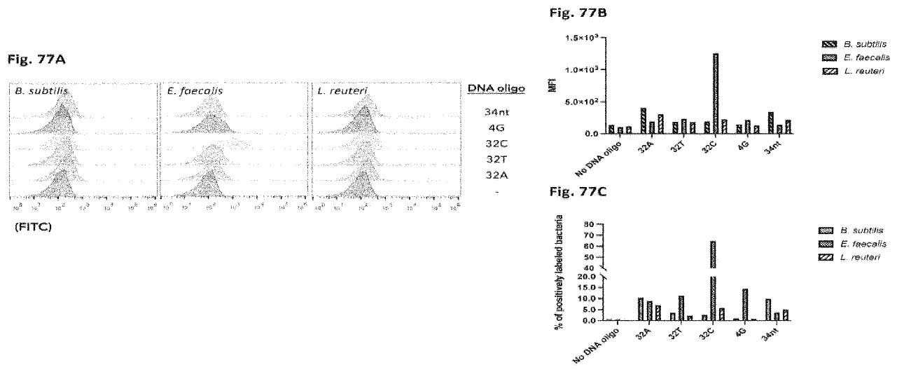

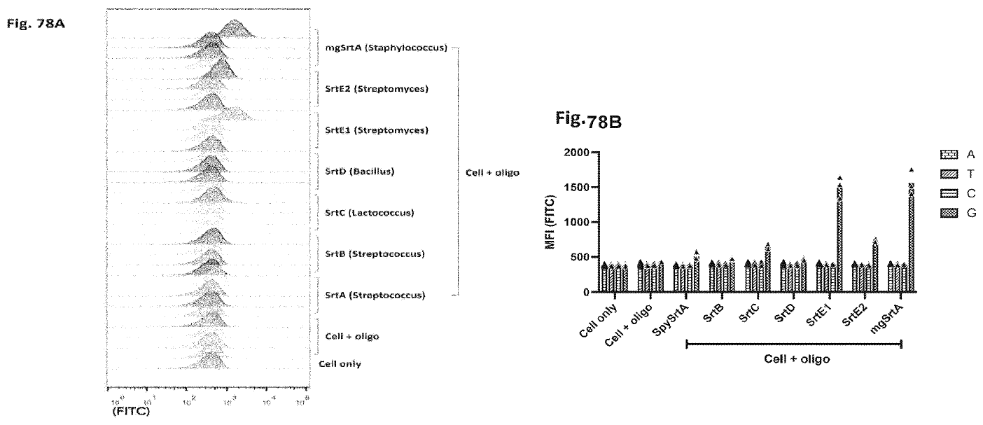

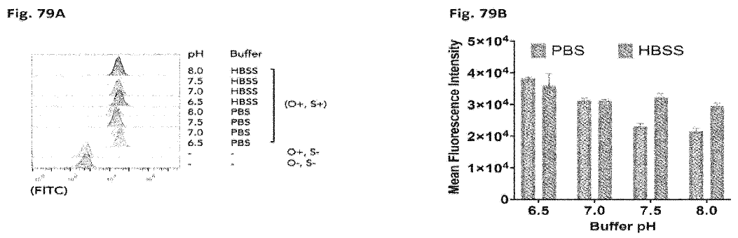

FIG. 15A shows confocal images showing the distribution of TAMRA signals in Jurkat cells. FIG. 15B shows confocal images showing the distribution of FITC signals in Jurkat cells. FIG. 15C shows confocal images showing the distribution of Cy5 signals in Jurkat cells. The materials, notations, and test conditions were the same as in FIG. 14.

FIG. 16A shows confocal images showing the distribution of TAMRA signals in MC-38 cells. FIG. 16B shows confocal images showing the distribution of FITC signals in MC-38 cells. FIG. 16C shows confocal images showing the distribution of Cy5 signals in MC-38 cells. The materials, notations, and test conditions were same as in FIG. 14.

FIG. 17A shows western blot images showing the reaction of two oligonucleotides and mgSrtA. The western blots showed that the intermediate products of mgSrtA and biotin-modified oligos were detected by an anti-biotin antibody, which indicated that oligonucleotides reacted with mgSrtA in a cell-free condition. FIG. 17B shows the sequence and modifications of each oligonucleotide (01, SEQ ID NO: 15 and 02, SEQ ID NO: 16).

FIG. 18A shows a bar plot showing the mean fluorescence intensity of K562 cells that were treated with a proteinase and then labeled with an FITC-modified oligonucleotide. The first two bars represented the blank cell control (oligo-, mgSrtA−) and the no-sortase control (oligo+, mgSrtA−). The “PBS” bar represented a sample without being treated by a proteinase but with the presence of sortase and oligos (oligo+ and mgSrtA+). The experiments were conducted in triplicates and the error bars were represented as +/−1 standard deviation. FIG. 18B shows multi-histograms of the FITC fluorescence signals from one representative replicate of the triplicates noted for FIG. 18A.

The sequence of the 3′-FITC modified oligonucleotide is set forth in SEQ ID 17:

GTGACTGGAGTTCAGACGTGTGCTCTTCCGATCTNNNNNNNNNNNNBAA
AAAAAAAAAAAAAAAAAAAAAAAAAAAAAA.

In the sequence of SEQ ID NO: 17, the letter N represented A, T, G or C nucleotide and the letter B represented C, G or T nucleotide.

FIG. 19 shows bar plots showing the mean fluorescence intensity of K562, Jurkat, and 293T cells that were treated with glycosidases in their respective enzyme reaction buffers and then labeled with an oligonucleotide (SEQ ID NO: 14) in presence of mgSrtA. A total of six groups of experiments was plotted. Within each group, an “NC”, a “HBSS buffer only,” and/or an “Enzyme reaction buffer only” sample were included as controls and compared with other samples that underwent different enzyme digestions. The experiments comprised two steps: (1) a glycosidases digestion step and (2) a nucleic acid labeling step. In the glycosidases digestion step, in the samples of “NC” and “HBSS buffer only,” the cells were incubated in an HBSS buffer but without a digestive enzyme; and in the “Enzyme reaction buffer only” samples, the cells were incubated in an enzyme reaction buffer but without a digestive enzyme. In the labeling step, in the samples of “HBSS buffer only,” the cells were incubated with mgSrtA and oligonucleotide; but in the samples of “NC”, no sortase enzyme or oligonucleotide were added. The samples of “Enzyme reaction buffer only” underwent similar treatments as the “HBSS buffer only” samples except that the samples of “Enzyme reaction buffer only” comprised an enzyme reaction buffer, not an HBSS buffer.

FIG. 20 shows multi-histograms of cells that were treated with heparinases and then labeled with oligonucleotides in presence of mgSrtA, showing one representative run from triplicate experiments in FIG. 19. FIG. 20A: K562 cells; FIG. 20B: Jurkat cells; FIG. 20C: 293T cells. Other notations were the same as in FIG. 19.

FIG. 21 shows multi-histograms of cells that were treated with chondroitinase ABC and then labeled with oligonucleotides in presence of mgSrtA, showing one representative run from triplicate experiments in FIG. 19. FIG. 21A: K562 cells; FIG. 21B: Jurkat cells; FIG. 21C: 293T cells. Other notations were the same as in FIG. 19.

FIG. 22 shows multi-histograms of cells that were treated with heparinase and chondroitinase combined digestion and then labeled with oligonucleotides in presence of mgSrtA, showing one representative run from triplicate experiments in FIG. 19. FIG. 22A: K562 cells; FIG. 22B: Jurkat cells; FIG. 22C: 293T cells. Other notations were the same as in FIG. 19.

FIG. 23 shows multi-histograms of cells that were treated with hyaluronidase digestion and then labeled with oligonucleotides in presence of mgSrtA, showing one representative run from triplicate experiments in FIG. 19. FIG. 23A: K562 cells; FIG. 23B: Jurkat cells; FIG. 23C: 293T cells. Other notations were the same as in FIG. 19.

FIG. 24 shows multi-histograms of cells that were treated with O-Glycosidase and PNGase F digestion and then labeled with oligonucleotides in presence of mgSrtA, showing one representative run from triplicate experiments in FIG. 19. FIG. 24A: K562 cells; FIG. 24B: Jurkat cells; FIG. 24C: 293T cells. Other notations were the same as in FIG. 19.

FIG. 25 shows multi-histograms of cells that were treated with Protein Deglycosylation Mix II digestion and then labeled with oligonucleotides in presence of mgSrtA, showing one representative from triplicate experiments in FIG. 19. FIG. 25A: K562 cells; FIG. 25B: Jurkat cells; FIG. 25C: 293T cells. Other notations were the same as in FIG. 19.

FIG. 26 shows comparisons between wild type (WT) SrtA and mgSrtA. FIG. 26A, FIG. 26B, and FIG. 26C show the labeling efficiencies of oligos and influences from the glycosidases as indicated in the figures. Other notations were the same as in FIG. 19. The amino acid sequence of the wild type SrtA is set forth in SEQ ID NO: 18:

KPHIDNYLHDKDKDEKIEQYDKNVKEQASKDKKQQAKPQIPKDKSKVAG
YIEIPDADIKEPVYPGPATPEQLNRGVSFAEENESLDDQNISIAGHTFI
DRPNYQFTNLKAAKKGSMVYFKVGNETRKYKMTSIRDVKPTDVGVLDEQ
KGKDKQLTLITCDDYNEKTGVWEKRKIFVATEVK.

FIG. 27 shows bar plots showing the mean fluorescence intensity of cells that were incubated with mgSrtA and an oligonucleotide (left, SEQ ID NO: 14) or a peptide (right) in K562 cells, Jurkat cells, Raji cells, 293T cells, and Hela cells. “NC” represented the incubation of cells, mgSrtA, and oligos. “PEG”, “Heparin,” and “ChonA Shark” represented the addition of 3000 ng/uL PEG8000, 300 ng/uL Heparin, and 300 ng/uL of Chondriotin sulfate Shark, respectively. The oligonucleotide was Cy5-modified and the peptide was FITC-modified. The peptide sequence is set forth in SEQ ID NO: 19: AALPET*G (FITC-Ahx-AALPET-(2-hydroxyacetic acid)-G).

FIG. 28 shows multi-histograms of cells labeled by an oligonucleotide (left panels, SEQ ID NO: 14) or a peptide (right panels, SEQ ID NO: 19), with the addition of PEG, heparin and chondroitin A Shark (ChonA Shark), respectively, showing one representative from triplicate experiments in K562, Jurkat, Raji, 293T and Hela in FIG. 27. The oligonucleotide was Cy5-modified and the peptide were FITC-modified. “NC” represented the incubation of cells, mgSrtA, and the oligonucleotide or the peptide.

FIG. 29 shows bar plots showing the mean fluorescence intensity of cells that were incubated with mgSrtA and an oligonucleotide (left, SEQ ID NO: 14) and a peptide (right, SEQ ID NO: 19) in K562 cells, Jurkat cells, and 293T cells. “NC” represented incubation of cells, mgSrtA and oligonucleotide or peptide. “Glucose”, “Glycogen”, “Heparin,” and “ChonA Shark” represented the addition of 300 ng/uL glucose, 300 ng/uL glycogen, 300 ng/uL Heparin, and 300 ng/uL of Chondriotin sulfate Shark, respectively.

FIG. 30 shows multi-histograms of cells labeled by an oligonucleotide (left panels, SEQ ID NO: 14) or a peptide (right panels, SEQ ID NO: 19), with the addition of glucose, glycogen, heparin, and chondroitin A Shark (ChonA Shark), respectively, showing one representative from triplicate experiments in K562 cells, Jurkat cells, and 293T cells in FIG. 29. The oligonucleotide was Cy5-modified and the peptide were FITC-modified. “NC” represented the incubation of cells, mgSrtA, and the oligonucleotide or the peptide.

FIG. 31 shows bar plots showing the mean fluorescence intensity of cells that were incubated with (A) an oligonucleotide (SEQ ID NO: 14) and (B) a peptide (SEQ ID NO: 19). “NC” represented the incubation of cells, mgSrtA, and oligos. “Heparin” and “Heparan sulfate” represented the addition of 300 ng/uL Heparin and Heparan Sulfate, respectively. The oligonucleotide was Cy5-modified and the peptide was FITC-modified.

FIG. 32 shows bar plots and multi-histograms of signals showing the labeling efficiencies of an oligonucleotide (SEQ ID NO: 13) and a peptide (SEQ ID NO: 19) across different cell lines. FIG. 32A shows normalized mean fluorescence intensity of oligonucleotides that were conjugated to K562, Jurkat, Raji, 293T, Hela, MC-38, and BaF3 cells. FIG. 32B shows normalized mean fluorescence intensity of peptides that were conjugated to these cells. The multi-histograms of FIG. 32C and FIG. 32D show the fluorescence signals from one representative replicate out of triplicate experiments.

FIG. 33 shows bar plots of oligonucleotide labeling on wildtype or various knock-out cells. The X-axis indicated the genotype of cells, and the y-axis indicated the labelling efficiencies represented by the mean fluorescence intensity (MFI). Two fluorescence modifications of the oligonucleotide by Cy5 (FIG. 33A) and TAMRA (FIG. 33B), respectively, were included. The Cy5-modified oligonucleotide of SEQ ID NO:14 and the TAMRA-modified oligonucleotide of SEQ ID NO: 12 were used.

FIG. 34 illustrates an example of a CellID oligonucleotide sequence design. From the most 5′ end to the most 3′ end, the oligonucleotide comprises a 22-nt anchor region enriched with guanine, a 35-nt PCR handle that is guanine-depleted, a 17-nt barcode region, and a capture sequence. The “capture sequence” can be designed as poly(A) or other specific sequence (e.g., GCTTTAAGGCCG (SEQ ID NO: 20), a capture sequence used from the 10× Genomics single cell platform) that can be used to enrich the CellID sequence.

The sequence of a 10× Capture Sequence 1 is set forth in SEQ ID NO: 20: GCTTTAAGGCCG;

GCTTTAAGGCCG;

The sequence of a 10× Capture Sequence 2 is set forth in SEQ ID NO: 21:

GCTCACCTATTAGC.

FIG. 35 shows a bar plot showing the mean fluorescence intensity collected from oligonucleotide (SEQ ID NO: 13) labeled cells in various buffers. The Y-axis of the bars represented the mean value, and the error bars represented the standard deviation from triplicate experiments.

FIG. 36A shows a line plot showing the mean fluorescence intensity collected from cells labeled with an oligonucleotide (SEQ ID NO: 12) under different temperatures and over the course of different length of incubation time. The multi-histogram shows the fluorescence signals from one representative replicate out of triplicate experiments performed in HBSS buffer. FIG. 36B shows multi-histograms showing one representative run from triplicate experiments of the labeling reactions performed at 4° C., RT, and 37° C. as noted for FIG. 36A.

FIG. 37 shows a bar plot showing the mean fluorescence intensity collected from cells labeled with an oligonucleotide (SEQ ID NO: 13) under different pH in PBS or HBSS buffer.

FIG. 38 shows multi-histograms showing that the addition of Ca²⁺ at different concentrations did not affect the labeling efficiencies of FITC-labeled oligonucleotide by the Ca²⁺-dependent (SEQ ID NO: 2) or the Ca²⁺-independent mgSrtA.

The amino acid sequence of Ca²⁺-independent mgSrtA is set forth in SEQ ID NO: 22:

KPHIDNYLHDKDKDEKIEQYDKNVKEQASKDKKQQAKPQIPKDKSKVAG
YIEIPDADIKEPVYPGPATREQLNRGVSFAKENQSLDDQNISIAGHTFI
GRPNYQFTNLKAAKKGSMVYFKVGNETRKYKMTSIRNVKPTAVGVLDEQ
KGKDKQLTLITCDDLNRETGVWETRKILVATEVK.

FIG. 39A shows a line plot of cell labeling efficiency across different concentrations of EDTA. The solid lines and the filled triangles represented the mean fluorescence intensity collected from cells labeled with an oligonucleotide (SEQ ID NO: 39) and then terminated with EDTA, and the intensities were marked on the left y-axis. The dashed lines and hollow triangles represented the percentage of positively labeled cells under the same conditions, and the percentages were marked on the right y-axis. Different EDTA concentrations were tested and both the Ca²⁺-dependent (SEQ ID NO: 2) and the Ca²⁺-independent mgSrtA (SEQ ID NO: 22) were used in the test. FIG. 39B shows multi-histograms showing the fluorescence signals from one representative replicate out of triplicate experiments illustrated in FIG. 39A.

FIG. 40A shows a line plot of cell labeling efficiency across different concentrations of an oligonucleotide and a peptide, respectively. The solid lines indicate the mean fluorescence intensity under different oligonucleotide or peptide concentrations, and the intensities were marked on the left y-axis. The dashed lines and hollow triangles indicate the percentage of positively labeled cells under the same conditions, and the percentages were marked on the right y-axis. FIG. 40B shows multi-histograms showing the fluorescence signals from one representative replicate out of triplicate experiments illustrated in FIG. 40A. In these experiments, the cells and the mgSrtA were incubated first and then the oligonucleotide or peptide was added. The peptide with N-terminal biotinylation (used in FIG. 40) is set forth in SEQ ID NO: 23: AALPET*G, in which the “*” denotes 2-hydroxyacetic acid.

The oligonucleotide with 3′-biotin (used in FIG. 40) is set forth in SEQ ID NO: 24: GGGGCGGGGTGGGGCGGGGAAATCATCTCAACCACTCACATCCACTACCAACACTCTH HCATCATCAATHHHHHGCTTTAAGG*C*C*G, in which the “*” denotes phosphorothioate.

FIG. 41A shows line plots indicating the mean fluorescence intensity and the percentage of positively labeled cells under different oligonucleotide concentrations. FIG. 41B shows multi-histograms showing the fluorescence signals from one representative replicate out of triplicate experiments, illustrated in FIG. 41A. In these experiments, the cells and the mgSrtA were incubated first and then the oligonucleotides were added. The experiments were conducted with the K562 and the Jurkat cell lines. The oligonucleotide of SEQ ID NO: 13 was used.

FIG. 42A shows line plots of cell labeling efficiency across different concentrations of an oligonucleotide (SEQ ID NO: 13), respectively. The solid lines indicate the mean fluorescence intensity under different oligonucleotide concentrations, and the intensities was marked on the left y-axis. The dashed lines and hollow triangles indicate the percentage of positively labeled cells under the same conditions, and the percentages were marked on the right y-axis. FIG. 42B shows multi-histograms showing the fluorescence signals from one representative replicate out of triplicate experiments, illustrated in FIG. 42A. In these experiments, the cells, the mgSrtA and the oligonucleotide or peptide were incubated together.

FIG. 43A shows line plots that compared the labeling signals between cells that were incubated with FITC labeled oligos with mgSrtA (mgSrtA+) or without mgSrtA (mgSrtA−). Both the mean fluorescence intensity (left y-axis) and the percentage of positively labeled cells (right y-axis) were shown. FIG. 43B shows multi-histograms showing the fluorescence signals from one representative replicate out of triplicate experiments, illustrated in FIG. 43A.

The oligonucleotide with 5′-FITC is set forth in SEQ ID NO: 25:

GGGGCGGGGGGGGAAAGTGACTGGAGTTCAGACGTGTGCTCTTCCGATC
THHHAAGTTCAAGTTAGACHHHAGCTTTAAGGCCGGTCCTAGCAA.

FIG. 44 shows comparisons of labeling efficiencies when using different sortase or sortase mutants to label K562 cells. FIG. 44A shows the mean fluorescence intensity of Cy5 signals from wild type sortase (WT, SEQ ID NO: 18), 5M, Chen2016, and mgSrtA. FIG. 44B shows multi-histograms showing the fluorescence signals from one representative replicate out of triplicate experiments illustrated in FIG. 44A. Vertical bars indicated the median from triplicates. The oligonucleotide of SEQ ID NO: 14 was used.

The amino acid sequence of 5M is set forth in SEQ ID NO: 26:

QAKPQIPKDKSKVAGYIEIPDADIKEPVYPGPATREQLNRGVSFAEENE
SLDDQNISIAGHTFIDRPNYQFTNLKAAKKGSMVYFKVGNETRKYKMTS
IRNVKPTAVEVLDEQKGKDKQLTLITCDDYNEETGVWETRKIFVATEV
K;

The amino acid sequence of Chen2016 is set forth in SEQ ID NO: 27:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGHTFI
GRPNYQFTNLKAAKKGSMVYFKVGNETRKYKMTSIRNVKPTAVGVLDEQ
KGKDKQLTLITCDDLNRETGVWETRKIFVATEVK.

FIG. 45 shows line plots showing the fluorescence signals collected from cells that were labeled with oligonucleotides (SEQ ID NO: 14) and cultured for 120 hrs. Two oligonucleotide concentrations (100 nM and 250 nM) and both mgSrtA+ and mgSrtA− were tested. FIG. 45A shows the mean fluorescence intensity and FIG. 45B shows the percentage of positively labeled cells. Experiments were conducted in triplicates and the mean and ±SD were illustrated.

FIG. 46 shows multi-histograms showing the fluorescence signals at different time points during the cell culture process from one representative replicate out of the three triplicate experiments as illustrated in FIG. 45. FIG. 46A shows signals collected from cells that were labeled with 100 nM oligonucleotides, and FIG. 46B shows signals collected from cells that were labeled with 250 nM oligonucleotides.

FIG. 47 shows confocal images across 120 hours during the cell culture process of the cells labeled by Cy5-oligo. K562 cells were labeled with 250 nM Cy5-modified (FIG. 47A) or FITC-modified (FIG. 47B) oligonucleotides with or without mgSrtA. Images of FIG. 47A were collected at 0 hrs, 12 hrs, 24 hrs, 48 hrs, 72 hrs, 96 hrs, and 120 hrs, and images of FIG. 47B were collected at 0 hrs, 24 hrs and 48 hrs. Images of FIG. 47C were orthogonal views of signals collected at 48 hrs after cells were labeled with Cy5-oligo, in which cell membrane was stained with TRITC (Tetramethylrhodamine). The orthogonal view is a commonly used image processing technique, in which an object was viewed from the x-y plane, the y-z plane, as well as the z-x plane. The oligonucleotide of SEQ ID NO: 14 was used in FIG. 47A and FIG. 47C, and the oligonucleotide of SEQ ID NO: 13 was used in FIG. 47B.

FIG. 48 shows fluorescence images of 293T cells at 48 hrs after labeled with a GFP plasmid at the presence of mgSrtA. The plasmid carries a GFP (green fluorescence protein) coding sequence. The green fluorescence indicated that the plasmids internalized into the cells, and the GFP protein was successfully expressed within the cell that was labeled with the GFP plasmid (white frame). The three columns represented images taken from cells with different treatment, and the two rows represented images taken from two microscope fields of view.

The sequence of the plasmid is set forth in SEQ ID NO: 28:

caactttgtatagaaaagttgctgtgccttctagttgccagccatctgttgtttgcccctcccccgtgccttccttgaccctggaaggtgccact
cccactgtcctttcctaataaaatgaggaaattgcatcgcattgtctgagtaggtgtcattctattctggggggtggggggggcaggacagcaag
ggggaggattgggaagacaatagcaggcatgctggggatgcggtgggctctatgggcgttacataacttacggtaaatggcccgcctggctgacc
gcccaacgacccccgcccattgacgtcaataatgacgtatgttcccatagtaacgccaatagggactttccattgacgtcaatgggtggagtatt
tacggtaaactgcccacttggcagtacatcaagtgtatcatatgccaagtccgccccctattgacgtcaatgacggtaaatggcccgcctggcat
tatgcccagtacatgaccttacgggactttcctacttggcagtacatctacgtattagtcatcgctattaccatgcctcgagggtacttatataa
gggggtgggggcgcgttcgtcctcagtcgcgatcgaacactcgagccgagcagacgtgcctacggaccgtctagacaagtttgtacaaaaaagca
ggctgccaccatgcccgccatgaagatcgagtgccgcatcaccggcaccctgaacggcgtggagttcgagctggtgggggcggagagggcacccc
cgagcagggccgcatgaccaacaagatgaagagcaccaaaggcgccctgaccttcagcccctacctgctgagccacgtgatgggctacggctt
ctaccacttcggcacctaccccagcggctacgagaaccccttcctgcacgccatcaacaacggggctacaccaacacccgcatcgagaagta
cgaggacggcggcgtgctgcacgtgagcttcagctaccgctacgaggccggccgcgtgatcggcgacttcaaggtggtgggcaccggcttcc
ccgaggacagcgtgatcttcaccgacaagatcatccgcagcaacgccaccgtggagcacctgcaccccatgggcgataacgtgctggtgggc
agcttcgcccgcaccttcagcctgcgcgacggcggctactacagcttcgtggtggacagccacatgcacttcaagagcgccatccaccccagc
atcctgcagaacgggggccccatgttcgccttccgccgcgtggaggagctgcacagcaacaccgagctgggcatcgtggagtaccagcacgc
cttcaagacccccatcgccttcgccagatctcgagctcgatgacgcaccaaggaagccctcgaggacgcgtaaaggtaccaaaggatcccgac
ctaccgacccagctttcttgtacaaagtggtgatggccggccgcttcgagcagacatgataagatacattgatgagtttggacaaaccacaact
agaatgcagtgaaaaaaatgctttatttgtgaaatttgtgatgctattgctttatttgtaaccattataagctgcaataaacaagttaacaacaa
caattgcattcattttatgtttcaggttcagggggaggtgtgggaggttttttaaagcaagtaaaacctctacaaatgtggtagcggccgcggcg
ctcttccgcttcctcgctcactgactcgctgcgctcggtcgttcggctgcggcgagcggtatcagctcactcaaaggcggtaatacggttatcca
cagaatcaggggataacgcaggaaagaacatgtgagcaaaaggccagcaaaaggccaggaaccgtaaaaaggccgcgttgctggcgtttttccat
aggctccgcccccctgacgagcatcacaaaaatcgacgctcaagtcagaggtggcgaaacccgacaggactataaagataccaggcgtttccccc
tggaagctccctcgtgcgctctcctgttccgaccctgccgcttaccggatacctgtccgcctttctcccttcgggaagcgtggcgctttctcata
gctcacgctgtaggtatctcagttcggtgtaggtcgttcgctccaagctgggctgtgtgcacgaaccccccgttcagcccgaccgctgcgcctta
tccggtaactatcgtcttgagtccaacccggtaagacacgacttatcgccactggcagcagccactggtaacaggattagcagagcgaggtatgt
aggcggtgctacagagttcttgaagtggtggcctaactacggctacactagaagaacagtatttggtatctgcgctctgctgaagccagttacct
tcggaaaaagagttggtagctcttgatccggcaaacaaaccaccgctggtagcggtggtttttttgtttgcaagcagcagattacgcgcagaaaa
aaaggatctcaagaagatcctttgatcttttctacggggtctgacgctcagtggaacgaaaactcacgttaagggattttggtcatgagattatc
aaaaaggatcttcacctagatccttttaaattaaaaatgaagttttaaatcaatctaaagtatatatgagtaaacttggtctgacagttaccaat
gcttaatcagtgaggcacctatctcagcgatctgtctatttcgttcatccatagttgcctgactccccgtcgtgtagataactacgatacgggag
ggcttaccatctggccccagtgctgcaatgataccgcgagacccacgctcaccggctccagatttatcagcaataaaccagccagccggaagggc
cgagcgcagaagtggtcctgcaactttatccgcctccatccagtctattaattgttgccgggaagctagagtaagtagttcgccagttaatagtt
tgcgcaacgttgttgccattgctacaggcatcgtggtgtcacgctcgtcgtttggtatggcttcattcagctccggttcccaacgatcaaggcga
gttacatgatcccccatgttgtgcaaaaaagcggttagctccttcggtcctccgatcgttgtcagaagtaagttggccgcagtgttatcactcat
ggttatggcagcactgcataattctcttactgtcatgccatccgtaagatgcttttctgtgactggtgagtactcaaccaagtcattctgagaat
agtgtatgcggcgaccgagttgctcttgcccggcgtcaatacgggataataccgcgccacatagcagaactttaaaagtgctcatcattggaaaa
cgttcttcggggcgaaaactctcaaggatcttaccgctgttgagatccagttcgatgtaacccactcgtgcacccaactgatcttcagcatcttt
tactttcaccagcgtttctgggtgagcaaaaacaggaaggcaaaatgccgcaaaaaagggaataagggcgacacggaaatgttgaatactcatac
tcttcctttttcaatattattgaagcatttatcagggttattgtctcatgagcggatacatatttgaatgtatttagaaaaataaacaaataggg
gttccgcgcacatttccccgaaaagtgccacctgacgtctaagaaaccattattatcatgacattaacctataaaaataggcgtatcacgaggc
cctttcgtcggcgcgccgcggccgc.

FIG. 49 shows plots showing the mean fluorescence intensity collected from different cell types after labeled with oligonucleotides (SEQ ID NO: 14). “Cell only” was included and served as the negative control. FIG. 49A shows the mean fluorescence intensity for various primary cells and FIG. 49B shows the mean fluorescence intensity for various immortalized cells. The measurements were collected from triplicates.

FIG. 50 shows multi-histograms showing the fluorescence signals from one representative replicate out of the three triplicate experiments as illustrated in FIG. 49.

FIG. 51 shows a schematic of CellID labeling for a 10× single cell RNA-seq (scRNA-seq) experiment. In step I, “labeling”: the cells in Samples 1 to 3 were labeled with different CellID oligos and each sample will hold a CellID with a unique sequence. In step II, “pooling”, cells from different samples were pooled. The pooled cells were subjected to scRNA-seq (e.g., 10× platform) as a single sample in step 3. In step 3, “scRNA-seq”, cells were lysated, and mRNA molecules were libraried and sequenced. During the process, CellIDs were also libraried together with mRNA molecules. The resulted data were demultiplexed in step 4 and information from individual samples were retrieved based on the identity of the respective CellIDs.

FIG. 52 lists the CellIDs that were used in a sample labeling for a scRNA-seq experiment. Each CellID represented one cell type. And the species that the cell line was derived from were also listed.

The sequence CellID CA11 is set forth in SEQ ID NO: 29:

GGGGCGGGGTGGGGCGGGGAAATCATCTCAACCACTCACATCCACTACC
AACACTCTHHCATATCACTAHHHHHBAAAAAAAAAAAAAAAAAAAAAAA
AA;

The sequence CellID CA12 is set forth in SEQ ID NO: 30:

GGGGCGGGGTGGGGGGGGGAAATCATCTCAACCACTCACATCCACTACC
AACACTCTHHCATCATCAATHHHHHBAAAAAAAAAAAAAAAAAAAAAAA
AA;

The sequence CellID CA13 is set forth in SEQ ID NO: 31:

GGGGCGGGGTGGGGGGGGGAAATCATCTCAACCACTCACATCCACTACC
AACACTCTHHTATACACCATHHHHHBAAAAAAAAAAAAAAAAAAAAAAA
AA;

The sequence CellID CA14 is set forth in SEQ ID NO: 32:

GGGGCGGGGTGGGGGGGGGAAATCATCTCAACCACTCACATCCACTACC
AACACTCTHHACATTACTACHHHHHBAAAAAAAAAAAAAAAAAAAAAAA
AA;

The sequence CellID CA15 is set forth in SEQ ID NO: 33:

GGGGCGGGGTGGGGGGGGAAATCATCTCAACCACTCACATCCACTACCA
ACACTCTHHTCAACTACATHHHHHBAAAAAAAAAAAAAAAAAAAAAAAA
A;

The sequence CellID CA16 is set forth in SEQ ID NO: 34:

GGGGGGGGGTGGGGGGGGGAAATCATCTCAACCACTCACATCCACTACC
AACACTCTHHAACATATCTCHHHHHBAAAAAAAAAAAAAAAAAAAAAAA
AA;

The sequence CellID CA17 is set forth in SEQ ID NO: 35:

GGGGGGGGGTGGGGGGGGGAAATCATCTCAACCACTCACATCCACTACC
AACACTCTHHTACCATACATHHHHHBAAAAAAAAAAAAAAAAAAAAAAA
AA;

The sequence CellID CA18 is set forth in SEQ ID NO: 36:

GGGGGGGGGTGGGGGGGGGAAATCATCTCAACCACTCACATCCACTACC
AACACTCTHHACTCACTTAAHHHHHBAAAAAAAAAAAAAAAAAAAAAAA
AA.

FIG. 53 shows tSNE plots of one scRNA-seq experiment multiplexed with eight samples, including five human cell lines (293T, K562, HeLa, Jurkat, and A549) and three mouse cell lines (Hepa1-6, MC-38, and C2C12). Cells were clustered and annotated according to their gene expression patterns. In each panel, cells carrying a particular CellID were highlighted, and the name of the cell type was listed at the top of each panel.

FIG. 54 shows that mammalian cells can be labeled by oligonucleotides mediated by mgSrtA. FIG. 54A. Oligonucleotides localized at the surface of K562 cells after mgSrtA-mediated cell labeling. FIG. 54B. Flow cytometry quantifications of the K562 cells labeled with FITC-modified DNA oligos. FIG. 54C. A summary plot of the K562 cells labeled with FITC-modified DNA oligos at different concentrations. FIG. 54D. Flow cytometry quantifications of the K562 cells labeled with Cy5-modified RNA oligos. FIG. 54E. A summary plot of the K562 cells labeled with Cy5-modified RNA oligos at different concentrations.

FIG. 55 shows that oligonucleotide binds with mgSrtA in vitro. FIG. 55A. Western Blotting (WB) showed that the 4G DNA oligo and mgSrtA yielded stronger binding product band compared to the 4A, 4T, and 4C oligos (the DNA oligos were biotinylated at the 5′ end). FIG. 55B. The WB bands shifted accordingly with the increase of the length of DNA oligo (the 4G, 6G, 8G, 15G, and 20G oligos were modified by 5′ biotin and 3′ FITC). The 4G DNA oligo (FIG. 55C) and the AALPETG (SEQ ID NO: 23) peptide (FIG. 55D) and mgSrtA mutants showed respective binding product bands. The mgSrtA-triple represents the mgSrtA mutant with H120A, C184A, and R197A mutations. FIG. 55E. The addition of Cu²⁺ strengthened the product bands of mgSrtA and the 4G DNA oligo.

FIG. 56 shows that mgSrtA bridged oligonucleotide on cell surface. FIG. 56A. Representative confocal images showing colocalization of oligonucleotide (Oligo-FITC) and mgSrtA (anti-His PE). The inset at the top-right of the “Merged” image is a magnified view of the single cell along corresponding grey lines. The nucleus was stained with Hoechst 33342. Arrow-pointed dots in the merged image indicates the overlap of mgSrtA and oligonucleotide. Scale bar, 20 μm. Fluorescence intensity profiles along the grey line in the merged image was shown at the bottom. FIG. 56B. The signals of the labeled oligonucleotide are positively correlated with the signals of anchored mgSrtA and its mutants on cell surface. FIG. 56C. A summary plot of the K562 cells labeled with Cy5-modified DNA oligos mediated by mgSrtA and mutants. FIG. 56D. A schematic flowchart of CRISPR screening to identify the cellular proteins involved or contributed to the mgSrtA-mediated oligonucleotide cell labeling. FIG. 56E. The top hits of CRISPR screening. Genes were ranked (x-axis) by p value (y-axis).

FIG. 57 shows that oligonucleotide binding is a previously unknown characteristic of wild-type sortase A. FIG. 57A. The 4G DNA oligo and wild-type (WT) sortase A and its mutants showed binding product bands. FIG. 57B. The addition of Cu² strengthened the product bands of WT sortase A and the 4G DNA oligo. FIG. 57C. In the sortase-mediated cell labeling, the signals of the labeled oligonucleotide are positively correlated with the signals of anchored WT sortase and its mutants on cell surface. FIG. 57D. A summary plot of the K562 cells labeled with Cy5-modified DNA oligos mediated by mgSrtA and mutants.

FIG. 58 shows Gram-positive bacteria labels oligonucleotides at their surface. FIG. 58A. S. aureus labels the 4-mer DNA oligos. B. A summary plot of the S. aureus labeled with the 4-mer DNA oligos. FIG. 58C. The DNA oligos could be labeled on the S. aureus but not E. coli. FIG. 58D. A summary plot of the S. aureus and E. coli oligo labeling. FIG. 58E. A variety of wild-type sortase were used to label oligonucleotide to K562 cells. FIG. 58F. A summary plot of the K562 cells labeled with Cy5-modified DNA oligos mediated by various WT sortase. In FIG. 58C and FIG. 58D, the sequence of the 34 nt is SEQ ID NO: 1.

FIG. 59 shows CellID application of mgSrtA-mediated cell labeling in multiplexed scRNA-seq. CellIDs accurately distinguished cells derived from eight samples.

FIG. 60A shows a reported crystal structure of wild-type sortase A and a peptide. FIG. 60B shows a docking simulation of the 4G DNA oligo and mgSrtA.

FIG. 61 shows an orthogonal view of mgSrtA-mediated cell labeling. The oligonucleotides localized at the surface of K562 cells after mgSrtA-mediated cell labeling. DAPI: Nuclear staining with NucBlue; Membrane: staining with CellMask Green; Oligonucleotide: visualized with the modified TAMRA.

FIG. 62 shows fluorescence signals of the positively labeled cells were detectable 120 hours post-labeling. FIG. 62A. The FITC-modified DNA oligo was used to label cells, and FITC signals were quantified within 24 hours at time intervals of 0.5 h, lh, 1.5 h, 2 h, 4 h, 8 h, 12 h, and 24 h. FIG. 62B. Summary plot of the MFI and the percentage of positively labeled cells within 24 hours. S: mgSrtA; O: DNA oligo.

FIG. 63A shows that both double-stranded (ds) and single-stranded (ss) DNA were labeled to cells mediated by mgSrtA. Equal moles of dsDNA and ssDNA were used in this quantification, in which each dsDNA molecular carries double amount of biotin modification than ssDNA. FIG. 63B shows a summary plot of the MFI.

FIG. 64 shows that mgSrtA mediates the Jurkat cell labeling by Cy5-modified RNA oligos. FIG. 64A. Flow cytometry quantifications of labeled Cy5-modified RNA oligos in different concentrations. FIG. 64B. Summary plot of the MFI.

FIG. 65 shows cell labeling is applicable to a variety of cell lines. Oligonucleotides were labeled to multiple cell types in the presence of mgSrtA. FIG. 65A. Flow cytometry quantifications of twelve cultured cell types. FIG. 65B. Summary plot of the percentage of positively labeled cultured cells. FIG. 65C. Summary plot of the normalized MFI of the cultured cells. FIG. 65D. Flow cytometry quantifications of seven cultured cells. FIG. 65E. Summary plot of the percentage of positively labeled primary cells. FIG. 65F. Summary plot of the normalized MFI of the primary cells.

FIG. 66 shows binding product bands of the 4G DNA oligo (FIG. 66A) and the AALPETG (SEQ ID NO: 23) peptide (FIG. 66B) with mgSrtA mutants. The mgSrtA-mono represents the mgSrtA mutant with N132A, K137A, and Y143A mutations.

FIG. 67 (coupled with FIG. 56A) shows that the overlap of mgSrtA and oligonucleotide. Scale bar, 10 m. Fluorescence intensity profiles along the grey line in the FIG. 67A was shown in the FIG. 67B.

FIG. 68 shows that mgSrtA mutations H120A (SEQ ID NO: 45), C184A (SEQ ID NO: 46), R197A (SEQ ID NO: 47), and mgSrtA-triple (SEQ ID NO: 48) could not label the peptide (SEQ ID NO: 19) to the cell surface of K562 cells.

FIG. 69 shows that the wild-type (WT), Cas9 knock in (WT-Cas9), and B4GALT7 knockout Hela cells were used to label oligonucleotide and AALPETG (SEQ ID NO: 19) peptide.

FIG. 70 shows that mgSrtA and heparin could yield product bands in vitro with the presence of Cu²⁺.

FIG. 71 shows that the addition of Cu²⁺, but not other metal cations this study tested, strengthened the product bands of mgSrtA and heparin.

FIG. 72 shows that biotin-modified heparin could be labeled to K562 cells mediated by mgSrtA (SEQ ID NO: 2), mgSrtA-L200F (SEQ ID NO: 50), and mgSrtA-triple (SEQ ID NO: 48). FIG. 72A. Flow cytometry quantifications of the labeled bio-modified heparin and sortase. FIG. 72B. Summary plot of the MFI.

FIG. 73 shows the top hits of CRISPR screening of AALPETG (SEQ ID NO: 19) cell labeling. Genes were ranked (x-axis) by p value (y-axis).

FIG. 74 shows representative confocal images showing colocalization of peptide (FITC-ETG) and mgSrtA (anti-His PE). FIG. 74A. Representative confocal images showing colocalization of AALPETG (SEQ ID NO: 19) peptide and mgSrtA. The inset at the top-right of the merged image is a magnified view of the single cell along corresponding the lines. The nucleus was stained with Hoechst 33342. The arrow-pointed dots in merged image indicates the overlap of mgSrtA and oligonucleotide. Scale bar, 20 μm. Fluorescence intensity profiles along the grey line in the merged image was shown in the bottom. FIG. 74B. The signals of the labeled peptides (FITC-ETG) are positively correlated with the signals of anchored mgSrtA (anti-His PE) and its mutants on cell surface.

FIG. 75 shows that the addition of Ca²⁺ strengthened the product bands of mgSrtA and peptide.

FIG. 76 shows that in the sortase-mediated cell labeling, the signals of the labeled oligonucleotide are positively correlated with the signals of WT and engineered sortase on cell surface. FIG. 76A. Flow cytometry quantifications of K562 cells labeled with oligonucleotide and sortase. FIG. 76B. A summary plot of flow cytometry quantifications.

FIG. 77 shows the signals of oligonucleotide labeled on Bacillus subitilis, Enterococcu, and Lactobacillaceae. FIG. 77A. Flow cytometry quantifications of bacteria labeled with FITC-modified oligonucleotide. FIG. 77B. A summary plot of the MFI of the K562 cells labeled with FITC-modified 4-mer DNA oligos. FIG. 77C. A summary plot of the positively labeled K562 cells with FITC-modified 4-mer DNA oligos (A: 4A oligo; T: 4T oligo; C: 4C oligo; G: 4G oligo).

FIG. 78 shows various wild-type sortase were used to label oligonucleotides to the surface of K562 cells. FIG. 78A. Flow cytometry quantifications of cells labeled with FITC-modified oligonucleotides mediated by various wild-type sortases. FIG. 78B. A summary plot of the K562 cells labeled with FITC-modified 4-mer DNA oligos (A: 4A oligo; T: 4T oligo; C: 4C oligo; G: 4G oligo).

FIG. 79 shows that the efficiencies of mgSrtA-mediated cell labeling were measured at different pH. FIG. 79A. Flow cytometry quantifications of K562 cells labeled with FITC-modified oligonucleotide under different pH. FIG. 79B. A summary plot of flow cytometry quantifications.

### DETAILED DESCRIPTION

All publications, including but not limited to patents and patent applications, cited in this specification are herein incorporated by reference as though fully set forth. If certain content of a reference cited herein contradicts or is inconsistent with the present disclosure, the present disclosure controls.

Any one embodiment of the disclosure described herein, including those described only in one section of the specification describing a specific aspect of the disclosure, and those described only in the examples or drawings, can be combined with any other one or more embodiment(s), unless explicitly disclaimed or improper.

### Definitions

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology are used.

As used in this specification and the appended claims, the singular forms “a,” “an,” and “the” include plural referents unless the content clearly dictates otherwise. Thus, for example, reference to “a cell” includes a combination of two or more cells, and the like.

The terms “polynucleotide,” “oligonucleotide,” “oligo,” “nucleic acid” and “nucleic acid molecule” are used interchangeably herein to refer to a polymeric form of nucleotides of any length, either ribonucleotides or deoxyribonucleotides. This term refers only to the primary structure of the molecule. A polynucleotide disclosed herein may be modified, e.g., with a labeling group such as a fluorophore, with a biotin, and with phosphorothioate. Such a modified polynucleotide may be referred to as a polynucleotide derivative. A polynucleotide derivative may comprise a modified purine or pyrimidine base.

A polynucleotide derivative includes a peptide nucleic acid. The term “peptide nucleic acid,” “oligo PNA,” or “PNA” are used interchangeably herein to refer to a polymer similar to DNA or RNA in structure. A PNA's backbone is typically composed of repeating N-(2-aminoethyl)-glycine units linked by peptide bonds. Purine and pyrimidine bases or any modified forms thereof are linked to the backbone by a bridge such as a methylene bridge (—CH₂—) and a carbonyl group (—(C═O)—). A PNA is considered as a derivative of nucleic acid.

The term “CellID” refers to an oligonucleotide sequence that can be used to label a cell and thus the labeled cell can be identified by the identity of the oligonucleotide sequence attached to the cell and/or internalized in the cell. The term “CellID” may also refer to a method of using such an oligonucleotide sequence design to label a cell.

For example, a “CellID” can refer to an oligonucleotide sequence design comprising a barcode of random sequences. For another example, a “CellID” can refer to an oligonucleotide sequence design comprising a barcode that does not comprise a random sequence (i.e., an oligonucleotide sequence design comprising a barcode of non-degenerate sequence).

For example, a CellID oligonucleotide sequence comprises an anchor region, wherein the anchor region is preferably guanine enriched.

For example, from the most 5′ end to the most 3′ end, a CellID oligonucleotide sequence comprises an anchor region that can be attached to a cell membrane, a PCR handle for amplification, a programmable region to distinguish individual cells (e.g., a barcode region), and a capture sequence for oligo enrichment. This CellID design can be used to identify cells, e.g., by single cell RNA-seq. Preferably, a CellID oligonucleotide sequence comprises an anchor region enriched with guanine (e.g., guanine represents more than 25% of the nucleotides in the nucleotide sequence), a PCR handle that is guanine-depleted (e.g., guanine represents less than 25% of the nucleotides in the nucleotide sequence), a programmable region to distinguish individual cells (e.g., a barcode region), and a capture sequence. The “capture sequence” can be designed as a poly(A) sequence or other specific sequence (e.g., GCTTTAAGGCCG (SEQ ID NO: 20), a capture sequence used from the 10× Genomics single cell platform) that can be used to enrich the CellID sequences.

“Barcoding” refers to a process of using a unique nucleotide sequence to label an entity and thus identify the entity. For example, “barcoding” can refer to a process of using a nucleic acid library of known sequences (nucleic acid barcodes) to label unknown samples and matching the barcode sequence of an unknown sample against the barcode library for identification.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like. A peptide disclosed herein may be modified, e.g., with a labeling group such as a fluorophore, a biotin, His tag, or phosphorothioate.

Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

As used herein, “percent (%) amino acid sequence identity” with respect to a peptide, polypeptide or protein sequence is defined as the percentage of amino acid residues in a candidate sequence that are identical with the amino acid residues in another peptide or polypeptide sequence, after aligning the sequences and introducing gaps, if necessary, to achieve the maximum percent sequence identity, and not considering any conservative substitutions as part of the sequence identity. Percent amino acid sequence identity in the current disclosure is measured using BLAST software. Those skilled in the art can determine appropriate parameters for measuring alignment, including any algorithms needed to achieve maximal alignment over the full length of the sequences being compared.

The term “polysaccharide,” “oligopolysaccharide,” “polycarbohydrates,” or “glycan” are used interchangeably herein to refer to polymeric carbohydrates composed of monosaccharide units bound together by glycosidic linkages. Polysaccharide can range in structure from linear to highly branched. Examples of polysaccharide includes glycosaminoglycan (GAG), e.g., heparin, heparan sulfate proteoglycan (HSPG), chondroitin sulfate proteoglycans (CSPG), heparan sulfate, chondroitin sulfate, or dermatan sulfate. Examples of polysaccharide also include storage polysaccharides such as starch, glycogen, and galactogen and structural polysaccharides such as cellulose and chitin. The term “glycan” may also be used to refer to the carbohydrate portion of a glycoconjugate, such as a glycoprotein (e.g., a glycoprotein comprising GAG), glycolipid, or a proteoglycan. The term “polysaccharide” as used herein also includes modified forms such as a polysaccharide modified by another group, such as sulfation, carboxymethylation, acetylation, and phosphorylation.

The term “subject” includes all animals such humans and other mammals.

The term “sortase” as used herein can be any wild type sortase or a variant of a wild type sortase, such as a mutated form of a wild type sortase, a sortase in the form of a fusion protein, or a sortase that is attached to a label or a tag.

The term “labeling,” “labeled,” or “label” means that a detectable or identifiable group is attached to an entity, via covalent and/or non-covalent bond(s). For example, a protein, a nucleic acid, or a polysaccharide can be labeled with a group such as a fluorophore, biotin, His tag, or phosphorothioate. For another example, a cell may be labeled (also referred to as “conjugated,” “anchored,” “ligated,” or “attached” herein) by a nucleic acid mediated (e.g., catalyzed) by a sortase. The nucleic acid may be internalized into the cells subsequently.

The term “sortagging,” “sortagged,” or “sortag” refers to sortase (e.g., SrtA)-mediated labeling of a cell covalently and/or non-covalently. For example, a nucleic acid can be labeled on a cell, mediated by a sortase, covalently and/or non-covalently.

### Novel Conjugation Reaction Mediated by Sortase and Conjugates Thereof

The inventors surprisingly discovered a novel reaction mediated by a sortase, wherein a nucleic acid or derivative thereof serves as a substrate for the sortase, which facilitates the ligation of the nucleic acid to a cell. In presence of a sortase, a nucleic acid or derivative thereof may be attached to the plasma membrane of a cell. An amino saccharide associated with the plasma membrane such as glycosaminoglycan (GAG) or a glycoprotein comprising GAG may be involved in such a conjugation reaction,

Examples of GAG includes heparin, heparan sulfate proteoglycan (HSPG), chondroitin sulfate proteoglycans (CSPG), heparan sulfate, chondroitin sulfate, and/or dermatan sulfate. Not wishing to be bound by theory, one or more glycans associated with the plasma membrane of a cell may sever as an anchoring factor that increases the local concentration of a sortase as disclosed herein, e.g., mgSrtA, and/or oligonucleotides, and thus enhances the ligation of the oligonucleotides and the plasma membrane.

In one embodiment, the disclosure provides a conjugate of a nucleic acid or derivative thereof and a sortase.

In one embodiment, the disclosure provides a conjugate of GAG, e.g., heparin, and a sortase as disclosed herein. For example, one or more GAG molecules in a plasma membrane of a cell may form a conjugate with a sortase as disclosed herein.

In one embodiment, the disclosure provides a conjugate of a nucleic acid or derivative thereof and a cell. In one embodiment, the disclosure provides a conjugate of a nucleic acid or derivative thereof and a cell via a sortase. For example, the sortase bridges the nucleic acid or derivative thereof and the cell in the conjugate. In one embodiment, the nucleic acid or derivative thereof is conjugated to the plasma membrane of the cell via a sortase. In one embodiment, the nucleic acid or derivative thereof is conjugated to a GAG, e.g., heparin, in the plasma membrane of the cell via a sortase.

The conjugation reaction can occur at a temperature that is suitable for a sortase and/or the cells. In one embodiment, conjugation reaction occurs at 4° C. to 40° C., such as 4° C. to 37° C., 4° C. to 25° C., or 18° C. to 25° C. In one embodiment, the conjugation reaction occurs at 4° C., at room temperature, or at 37° C.

In one embodiment, the conjugation reaction occurs in presence of a metal ion, such as Cu²⁺, wherein the metal ion improves the reaction.

The conjugation reaction can occur at a pH that is suitable for a sortase and/or cells. In one embodiment, the conjugation reaction occurs at a pH from 4 to 8, e.g., 6 to 8, preferably 6.5 to 8.

In one embodiment, the conjugation reaction lasts for about 1 to 30 min, e.g., 5-10 min or 5 to 20 min.

The sortase used in the conjugation reaction or in the conjugate disclosed herein can be any sortase, such as any sortase disclosed herein. For example, the sortase can be sortase A, sortase B, sortase C, sortase D, sortase E, sortase F, or a variant of any of these sortases. In one embodiment, the sortase is mgSrtA. In one embodiment, the sortase is selected from a wild type sortase, a 5M sortase, a Chen2016 sortase, and mgSrtA.

In one embodiment, the sortase used in the conjugation reaction or in the conjugate disclosed herein is selected from SEQ ID NOs: 2, 18, 22, 27, 45-58, and 64-67, and a sortase having an amino acid sequence with at least 80%, 85%, 90%, 95%, 96%, 97%, 98%, 99%, or 100% identity to any one of SEQ ID NOs: 2, 18, 22, 27, 45-58, and 64-67.

In one embodiment, the sortase used in the conjugation reaction or in the conjugate disclosed herein is selected from SpySrtA, SrtE1, SrtE2, SrtF, SrtD, and mgSrtA and variants thereof.

The nucleic acid or derivative thereof suitable for the conjugation reaction or the conjugate can be DNA or RNA, or a derivative of DNA or RNA. For example, the derivative can be DNA or RNA modified with a labeling group, such as a fluorophore, a biotin, or phosphorothioate. The derivative can also be DNA or RNA comprising a modified purine or pyrimidine base. In another example, the derivative can be a PNA or a derivative of PNA.

The nucleic acid or derivative thereof suitable for the conjugation reaction or the conjugate may be double stranded or single stranded. The nucleic acid or derivative thereof can be of any length, such as 1 to 4000 nucleotides, 4-500 nucleotides, 10-200 nucleotides, etc.

In one embodiment, the polynucleotide used in the conjugation reaction or in the conjugate comprises a sequence that is a guanine-enriched. For example, the sequence comprises guanines that represent more than 25%, e.g., 30%, 40%, 50%, 60%, 70%, 80%, 90%, or 100%, of the nucleotides in the sequence.

Cells that can be used in a conjugation reaction or in the conjugate as disclosed herein can be any cells, such as bacterial cells, yeast cells, or any mammalian cells. The cells include any wild type cells or any genetically modified cells such as knock-out cells.

Cell types suitable for the conjugation reaction or the conjugate as disclosed herein can have a broad range of characteristics including both cultured cells and primary cells. For example, the cells can be primary cells or immortalized cells. The cells can be cancer cell lines, stem cells, mice spleen cells. Examples of primary cells include thymus cells, kidney cells, liver cells, lung cells, bone marrow cells, or the red blood cell cells. Examples of cells include K562 cells, Jurkat cells, 293T cells, Raji cells, Hela cells, MC-38, and BaF3.

In one embodiment, the cells suitable for the conjugation reaction or the conjugate as disclosed herein are cells in vivo, such as those in a subject.

The conjugation reaction as described herein can be carried out in vitro or in vivo.

In one embodiment, the conjugation reaction is carried out by incubating a mixture comprising three components, a nucleic acid or a derivative, a cell (or GAG), and a sortase, for a suitable period of time, such as about 1 to 30 min. Any two of the three components can be included first for a suitable period of time (such as 1 min to 15 min), and then the third component can be added and incubated with the mixture of the first two components for another suitable period of time (such as 1 min to 15 min).

In one embodiment, the conjugation reaction is carried out by incubating a mixture of a nucleic acid and cells for a suitable period of time (e.g., 5 to 10 mins) at a temperature ranging from 4° C. to 40° C., then a sortase is added to the mixture, and then the resulting mixture is included for another suitable period of time (e.g., 5 to 10 mins) at a temperature ranging from 4° C. to 40° C. This order of mixing the polynucleotide, sortase, and cell is referred to as the “Oligo-1st” or “Oligo-first” approach. For instance, in an “Oligo-1st” labeling experiment, 0.5 million cells are firstly incubated with oligos at 37° C. for 5 mins, followed by the addition of mgSrtA to a 20 μM final concentration and incubated at 37° C. for another 10 mins.

In one embodiment, the conjugation reaction is carried out by incubating a mixture of cells and a sortase for a suitable period of time (e.g., 5 to 10 mins) at a temperature ranging from 20° C. to 40° C., then a polynucleotide is added to the mixture, and then the resulting mixture is included for another suitable period of time (e.g., 5 to 10 mins) at a temperature ranging from 20° C. to 40° C. This order of mixing the cells, sortase, and polynucleotide is referred to as the “Enzyme-1st” or “Enzyme-first” approach. For instance, in an “Enzyme-1st” labeling experiment, 0.5 million cells were firstly incubated with 20 μM mgSrtA at 37° C. for 5 mins, followed by the addition of oligos and incubated at 37° C. for another 10 mins.

In one embodiment, the conjugation reaction is carried out by incubating a mixture of cells, a sortase, and a polynucleotide for a suitable period of time (e.g., 1 to 30 mins) at a temperature ranging from 4° C. to 40° C. This order of mixing the cells, sortase, and polynucleotide is referred to as the “Together” approach.

In one embodiment, the present disclosure provides a method of labeling cells with a programmable nucleic acid or derivative thereof such as DNA, RNA, or PNA. Such a method can be used to identify or barcode unique cells in a cell population or mixture of cells. For example, cells can be barcoded by CellID nucleic acids as disclosed herein and then identified subsequently by sequencing, e.g., single cell RNA-seq.

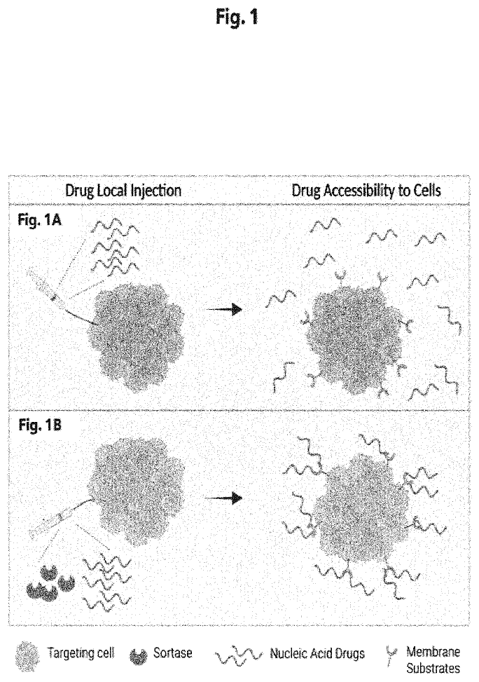

In one embodiment, a nucleic acid ligated to the cell membrane can subsequently enter the cells. Thus, the ability of anchoring a nucleic acid or derivative thereof to cell membranes can provide a method of delivering nucleic acid drugs of gene therapy or vaccines to a subject, such as a human patient. The nucleic drug or vaccine can be designed to comprise a suitable anchoring region (e.g., with a guanine enriched region) that can be anchored to cell membranes facilitated by a sortase. Such a nucleic drug or vaccine can subsequently enter the cells so as to exert therapeutic effect as illustrated in FIGS. 1-5.

### Sortases

The sortase used in the conjugation reaction or conjugate disclosed herein can be any naturally occurring sortase or functional variant thereof. Sortase was first discovered as a group of proteins that modify surface proteins by recognizing and cleaving a carboxyl-terminal sorting signal. For most substrates of sortase enzymes, the recognition signal consists of the motif LPXTG (Leu-Pro-any-Thr-Gly), then a highly hydrophobic transmembrane sequence, followed by a cluster of basic residues such as arginine. Cleavage occurs between the Thr and Gly, with transient attachment through the Thr residue to the active site Cys residue, followed by transpeptidation that attaches the protein covalently to cell wall components.

There are at least six classes of Sortases, including Sortase Class A, B, C, D, E, and F, as shown in the table below ¹¹.

TABLE 1
Sortase classes, substrates and substrate recognition motifs
with species specificity

| Sortase | | | |
|---|---|---|---|
| class | Motif | Substrates | Species |
| A | LPXTG | Surface | All low GC content Gram- |
| | | proteins | positive bacteria |
| B | NP(Q/K)TN | Haem | Low GC content Gram-positive |
| | | acquisititon | bacilli and cocci |
| | | proteins | |
| C | (I/L)(P/A)XTG | Pilin subunits | Both low and high GC content |
| | | | Gram-positive bacteria |
| D | LPNTA | Endospore | Bacillus species |
| | | envelope | |
| | | proteins | |
| E | LAXTG | Pili | High GC content |
| | | | Gram-positive bacteria |
| F | — | — | Actinobacteria |

As noted above, a diverse range of sortase variants have been developed, including a sortase variant (eSrtA, 5M) ⁷, Srt7M⁶, the Chen group's evolved variant based on the 5M variant ⁸, the Chen group's “promiscuous” SrtA variant, mgSrtA ⁹, and an LMVGG (SEQ ID NO: 69)-recognizing SrtA variant¹⁰.

In one embodiment, mgSrtA is used to ligate nucleic acids or derivatives thereof to the plasma membrane of live cells covalently and efficiently.

In one embodiment, the sortase used in the conjugation reaction disclosed herein is selected from SEQ ID NOs: 2, 18, 22, 27, 45-58, and 64-67, and a sortase having an amino acid sequence with at least 80%, 85%, 90%, 95%, 96%, 97%, 98%, 99%, or 100% identity to any one of SEQ ID NOs: 2, 18, 22, 27, 45-58, and 64-67.

In one embodiment, the sortase used in the conjugation reaction disclosed herein is selected from SpySrtA, SrtE1, SrtE2, SrtF, SrtD, and mgSrtA.

### Methods of Use

The discovery that a nucleic acid or derivative thereof can be ligated to a cell mediated by a sortase has broad range of uses, such as, as research tools (e.g., barcoding cells) or for disease diagnosis or medical treatment (e.g., drug delivery). Barcoding and drug delivery methods utilizing the conjugation reaction disclosed herein are exemplified below.

### Barcoding

A nucleic acid or derivative thereof can be ligated to a cell and provides an additional layer of information for identifying the labeled cell, wherein the ligated nucleic acid or derivative thereof can be characterized and quantified by DNA sequencing (e.g., by high throughput sequencing). This layer of information can be directly used as a cell identifier. Such a cell identifier is referred to as a CellID oligonucleotide or simply CellID. The term “CellID” may also refer to a method of using such an oligonucleotide sequence design to label a cell.

In one embodiment, a CellID oligonucleotide comprises a barcode sequence. For example, from the most 5′ end to the most 3′ end, the oligonucleotide sequence comprises an anchor region (e.g., ˜4 to ˜2000 nt, preferably 4-30 nt), a PCR handle (e.g., ˜18 to ˜40 nt), a barcode region (e.g., 1 to 50 nt, depending on the coding complexity (which can be calculated as 4ⁿ) needed), and a capture sequence. For example, the anchor region may be 22-nt enriched with guanine, the PCR handle may be 35-nt that is guanine-depleted, and the barcode region may be 17-nt. See FIG. 34. The “capture sequence” may be designed as poly(A) or other specific sequence (e.g., GCTTTAAGGCCG (SEQ ID NO: 20), a capture sequence used from the 10× Genomics single cell platform) that can be used to enrich the CellID sequences. The CellID information, together with the other molecular phenotypes of the cells, can be used to characterize cells. The other molecular phenotypes of the cells include the genome DNA sequences, the RNA expression levels, and the DNA methylation profiles, etc. The characterization of the cells can be at a bulk cell level or at a single cell level. For example, multiple samples representing different treatment conditions can be labeled by respective oligonucleotides and mixed as a single sample for single cell RNA-seq as illustrated by FIG. 51. This method can eliminate batch effects (e.g., variations) across samples and decrease costs.

The CellID oligonucleotides can also be used to label cells that participate in certain biological processes in an area in vivo. For example, by injecting a sortase (e.g., mgSrtA) and different oligonucleotides into a tumor at multiple time points, tumor infiltrated lymphocytes (TILs) can be labeled. The labeled TILs can be isolated by using a cell isolation technique, e.g., cell sorting, and analyzed for their presence at different timepoints.

### Drug Delivery

Sortase-mediated oligonucleotide labeling of cells can increase the local concentration of the oligonucleotide at or around the cells, by rapidly anchoring oligonucleotide to the cell membrane. Since the anchored oligonucleotides can subsequently be internalized by cells, external nucleic acids or derivatives (e.g., a nuclei acid drug, vaccine, or a bioconjugate comprising a nucleic acid and a treating modality such a small molecule or peptide) in various formats can be efficiently delivered into cells and participate in diverse downstream biological processes.

FIG. 1 illustrates a comparison of local distributions of a nucleic acid drug after local injection of the drug, without (up panel) or with (bottom panel) a sortase. As illustrated, the sortase rapidly mediates the conjugation between the nucleic acid drug and the cell membrane before diffusion of the nucleic acid drug molecules, resulting in concentration of the nucleic acid drug molecules on the cell. When no sortase is involved, the nucleic acid drug molecules diffuse away from the cell.

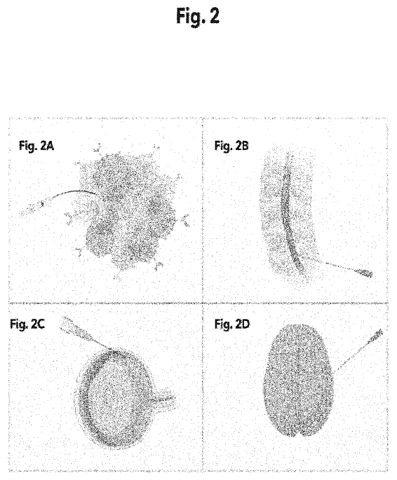

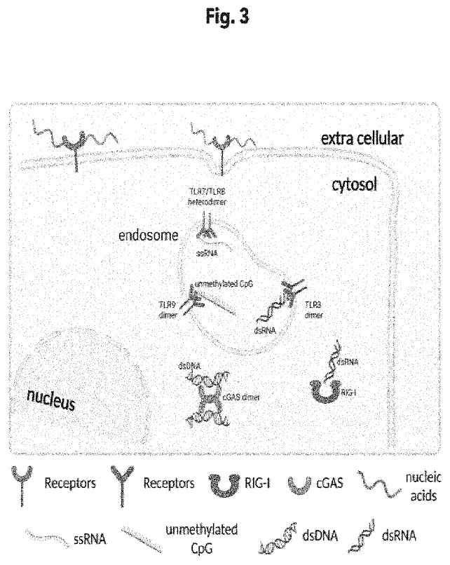

Injection locations that are suitable for gene therapy are applicable for injection of a nucleic acid drug with a sortase. As illustrated in FIG. 2, nucleic acid drugs or their derivatives can be locally injected with a sortase to various sites such as (A) tumor sites; (B) epidural sites; (C) intravitreal sites; or (D) intracerebral sites. Once a nucleic drug or its derivative enters the cells, it can exert therapeutic effect as illustrated in FIGS. 3-5.

Nucleic acid drugs function as ligands to bind with intracellular receptors and transduce downstream signals ¹²⁻¹⁵ The internalized nucleic acid drugs can result in downstream signaling transduction and be sensed by various intracellular receptors. For example, the receptors can be Toll-like receptors, cGAS, or RIG-I etc (FIG. 3).

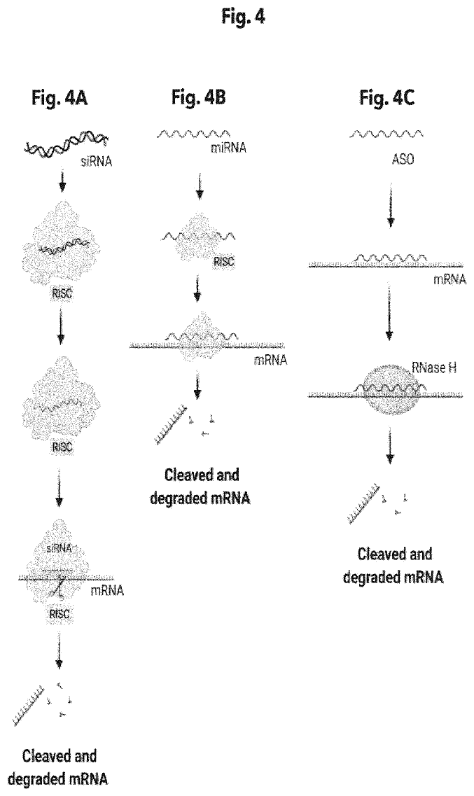

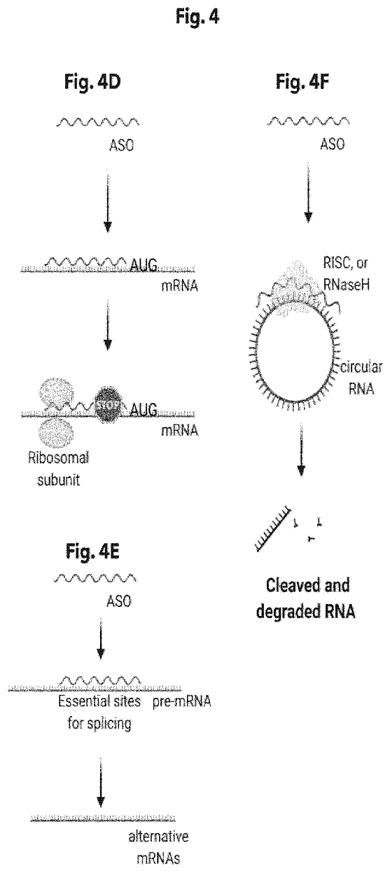

Nucleic acid drugs may function through sequence complement ^{16, 17} Nucleic acid drugs can exert their functions by sequence hybridization after internalized into cells to which they are conjugated. FIG. 4 illustrates several examples of nucleic acid drugs and how they function. FIG. 4A, FIG. 4B, and FIG. 4C illustrate that nucleic acid drugs hybridize with targeting mRNA, and result in degradation of the targeting mRNA. FIG. 4D and FIG. 4E illustrate that nucleic acid drugs serve as steric-blocking oligonucleotides to regulate the expression of targeting mRNA without degradation of the mRNA. FIG. 4F illustrates that nucleic acid drugs can also target circular RNA by sequence hybridization and cause circular RNA degradation.

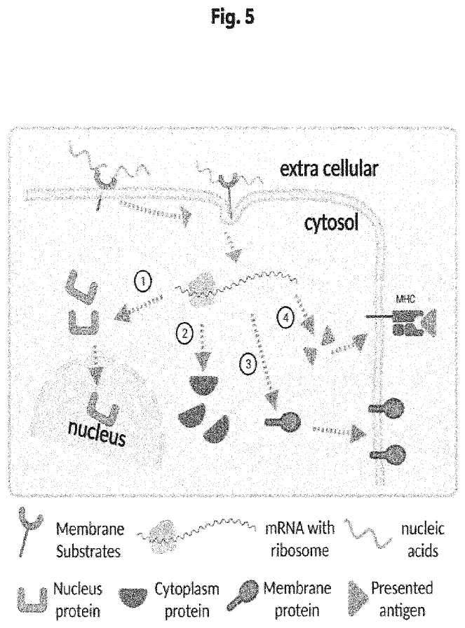

Nucleic acid drugs can serve as mRNA templates to produce functioning proteins ^{16, 18} (FIG. 5). As illustrated in FIG. 5, nucleic acid drug molecules are conjugated to the cell membrane of a cell facilitated by a sortase and then are internalized into cell. After released to the cytoplasm, the nucleic acid drug can serve as an mRNA template, and a corresponding protein is translated. The resulted protein can serve as a nucleus protein to orchestrate the transcriptional programs, stay in cytoplasm, be transported to the cytoplasm membrane, or be presented extracellularly by MHC complex.

Nucleic acids can also be conjugated with circulating cells. In these cases, circulating cells can serve as vehicles traveling through the body, and the conjugated oligonucleotides can serve as cargos for therapeutic purposes ¹⁹. The nucleic acids could be drugs by themselves or could be part of bioconjugates comprising a treating modality, and serve as delivery vehicles.

Nucleic acid drugs disclosed herein can also be modified, as other nucleic acid drugs, to enhance favorable drug properties for, e.g., delivery and durability. Common modifications include chemical modification, backbone modification, nucleobase modification, terminal modification, ribose sugar modification, bridged nucleic acids, and nucleic acid analogs (e.g., PNA) ¹⁶.

### EXAMPLES

The following examples are provided to describe the disclosure in greater detail. They are intended to illustrate, not to limit, the disclosure.

### Example 1: Cell Culture

K562 and Jurkat were cultured in RPMI1640 (Sigma R8758) supplemented with 10% fetal bovine serum, 1% penicillin/streptomycin. 293T, Hela, A549, MC-38, Hepa1-6 and C2C12 were cultured in DMEM (Sigma D6429) supplemented with 10% fetal bovine serum (Gemini 900-108) and 1% penicillin/streptomycin (Gibco 15140-122). H1 was cultured in mTeSR™1 Basal Medium (STEMCELL 85851) with 1×mTeSR™1 supplement (STEMCELL 85852).

### Example 2: Preparation of DNA Oligo, RNA Oligo, and Double-Stranded DNA

Oligonucleotides were ordered from General Biol (Anhui, China), Genscript (Nanjing, China) and Genewiz (Suzhou, China). Peptides were ordered from Scilight Biotechnology (Beijing, China). A powder of Cy5-modified RNA oligo was diluted with RNase free H₂O and aliquoted in −80° C. freezer.

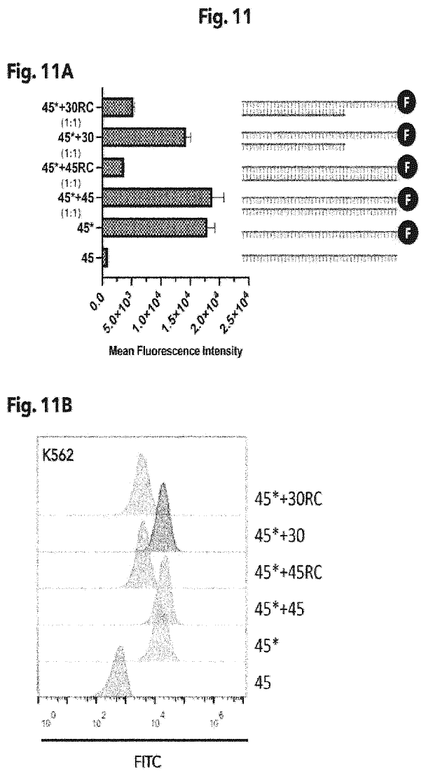

A FITC-modified 45-nt oligo (denoted as 45* in FIG. 11) was mixed with the equal molar of its complementary chain or itself without modification. Then the mixtures were heated at 95° C. for 5 mins and returned to room temperature. FITC-modified strands in ssDNA, dsDNA, partial dsDNA, and the mixtures of ssDNAs at a final concentration of 50 nM respectively were incubated with 0.5 million K562 in the presence of 20 uM mgSrtA at 37° C. for 10 mins.

The biotin-modified double-stranded DNA (denoted as dsDNA_18 bp dsDNA_207 bp, dsDNA_213 bp, and dsDNA_302 bp in FIG. 63B) were PCR products amplified from a plasmid.

The sequence of dsDNA_118 bp is set forth in SEQ ID NO: 59:

gacagatagcaccaggtcagactgggaagatagcggattacaactataa
gttgcccgatgattttacggggtgcgtaatcgcatggaactcaaacaac
ctcgactccaaagtaggtgg

The sequence of dsDNA_302 bp is set forth in SEQ ID NO: 60:

gtgttacggcgtttctccaacgaagctgaatgatctctgttttacgaac
gtgtatgctgactctttcgttatacggggggacgaagtgagacagatag
caccaggtcagactgggaagatagcggattacaactataagttgcccga
tgattttacggggtgcgtaatcgcatggaactcaaacaacctcgactcc
aaagtaggtggtaattataattacttgtatcgcctgtttcgaaagagca
atttgaagccttttgagcgggatatttcaaccgaaatttaccaagcagg
cagtacgc

The sequence of dsDNA_213 bp is set forth in SEQ ID NO: 61:

gacagatagcaccaggtcagactgggaagatagcggattacaactataa
gttgcccgatgattttacggggtgcgtaatcgcatggaactcaaacaac
ctcgactccaaagtaggtggtaattataattacttgtatcgcctgtttc
gaaagagcaatttgaagccttttgagcgggatatttcaaccgaaattta
ccaagcaggcagtacgc

The sequence of dsDNA_207 bp is set forth in SEQ ID NO: 62:

gtgttacggcgtttctccaacgaagctgaatgatctctgttttacgaac
gtgtatgctgactctttcgttatacggggggacgaagtgagacagatag
caccaggtcagactgggaagatagcggattacaactataagttgcccga
tgattttacggggtgcgtaatcgcatggaactcaaacaacctcgactcc
aaagtaggtgg

The sequence of ssDNA_86 nt is set forth in SEQ ID NO: 63:

GGGGGGGGGTGGGGGGGGGAAATCATCTCAACCACTCACATCCACTACC
AACACTCTHHCATCATCAATHHHHHGCTTTAAGGCCG

### Example 3: Sortase Protein Expression and Purification

The DNA sequences of a wild type sortase (SEQ ID NO: 18), mgSrtA (Ca²⁺-dependent, SEQ ID NO: 2), mgSrtA (Ca²⁺-independent, SEQ ID NO: 22), Chen2016 (SEQ ID NO: 27), mgSrtA-H120A (SEQ ID NO: 45), mgSrtA-C184A (SEQ ID NO: 46), mgSrtA-R197A (SEQ ID NO: 47), mgSrtA-triple (SEQ ID NO: 48), WT-F200L (SEQ ID NO: 49), 5M (SEQ ID NO: 50), mgSrtA-L200F (SEQ ID NO: 51), WT-mono (SEQ ID NO: 52), SpySrtA (SEQ ID NO: 53), SrtB (SEQ ID NO: 54), SrtC (SEQ ID NO: 55), SrtD (SEQ ID NO: 56), SrtE1 (SEQ ID NO: 57), SrtE2 (SEQ ID NO: 58), mgSrtA-DN59 (SEQ ID NO: 64), mgSrtA-K134A (SEQ ID NO: 65), mgSrtA-mono (SEQ ID NO: 66), SrtF (SEQ ID NO: 67) were cloned into pET-28a backbone with a N-terminal 6×His tag. The vector containing the DNA sequence 5M (SEQ ID NO: 26) was ordered from Addgene (Catalog No. 75144). The vector was transformed and expressed in E. coli BL21 (DE3). IPTG (0.2 mM) was added to each liter of E. coli when the OD600 reached 0.6. The cultures continued growing overnight at 18° C. before harvested by centrifugation. The cell pellet was resuspended in 40 mL lysis buffer (20 mM Tris-HCl, pH 7.8, 500 mM NaCl) supplemented with protease inhibitors. The lysate was sonicated for 4 s followed by 4 s resting and lasted 150 cycles at 35% vibration amplitude with one-half inch probe on Branson SFX550. The lysate after sonication was centrifuged and the supernatant was filtered using a 0.45 um filter (Millipore SLHVR33RB) before loaded into a gravity column with 2.5 mL Ni-NTA Agarose (Qiagen 1018244). The column was washed with 20 mL washing buffer (20 mM Tris-HCl, pH 7.8, 500 mM NaCl, 40 mM imidazole), and the target protein was eluted by 40 mL elution buffer (20 mM Tris-HCl, pH 7.8, 500 mM NaCl and 250 mM imidazole). The Amicon Ultra-15 Centrifugal Filters can be applied when a small volume is desired. The purified protein was then stored at −80° C. in 10% glycerol as stock.

The sequence of mutant mgSrtA-H120A is set forth in SEQ ID NO: 45:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGATFI
GRPNYQFTNLKAAKKGSMVYFKVGNETRKYKMTSIRNVKPTAVGVLDEQ
KGKDKQLTLITCDDLNRETGVWETRKILVATEVK

The sequence of mutant mgSrtA-C184A is set forth in SEQ ID NO: 46:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGHTFI
GRPNYQFTNLKAAKKGSMVYFKVGNETRKYKMTSIRNVKPTAVGVLDEQ
KGKDKQLTLITADDLNRETGVWETRKILVATEVK

The sequence of mutant mgSrtA-R197A is set forth in SEQ ID NO: 47:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGHTFI
GRPNYQFTNLKAAKKGSMVYFKVGNETRKYKMTSIRNVKPTAVGVLDEQ
KGKDKQLTLITCDDLNRETGVWETAKILVATEVK

The sequence of mutant mgSrtA-triple is set forth in SEQ ID NO: 48:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGATFI
GRPNYQFTNLKAAKKGSMVYFKVGNETRKYKMTSIRNVKPTAVGVLDEQ
KGKDKQLTLITADDLNRETGVWETAKILVATEVK

The sequence of mutant WT-F200L is set forth in SEQ ID NO: 49:

KPHIDNYLHDKDKDEKIEQYDKNVKEQASKDKKQQAKPQIPKDKSKVAG
YIEIPDADIKEPVYPGPATPEQLNRGVSFAEENESLDDQNISIAGHTFI
DRPNYQFTNLKAAKKGSMVYFKVGNETRKYKMTSIRDVKPTDVGVLDEQ
KGKDKQLTLITCDDYNEKTGVWEKRKILVATEVK

The sequence of mutant 5M is set forth in SEQ ID NO: 50:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGHTFI
DRPNYQFTNLKAAKKGSMVYFKVGNETRKYKMTSIRNVKPTAVGVLDEQ
KGKDKQLTLITCDDYNEETGVWETRKIFVATEVK

The sequence of mutant mgSrtA-L200F is set forth in SEQ ID NO: 51:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGHTFI
GRPNYQFTNLKAAKKGSMVYFKVGNETRKYKMTSIRNVKPTAVGVLDEQ
KGKDKQLTLITCDDLNRETGVWETRKIFVATEVK

The sequence of mutant WT-mono is set forth in SEQ ID NO: 52:

KPHIDNYLHDKDKDEKIEQYDKNVKEQASKDKKQQAKPQIPKDKSKVAG
YIEIPDADIKEPVYPGPATPEQLNRGVSFAEENESLDDQNISIAGHTFI
DRPNYQFTALKAAAKGSMVAFKVGNETRKYKMTSIRDVKPTDVGVLDEQ
KGKDKQLTLITCDDYNEKTGVWEKRKIFVATEVK

The sequence of SpySrtA is set forth in SEQ ID NO: 53:

SVLQAQMAAQQLPVIGGIAIPELGINLPIFKGLGNTELIYGAGTMKEEQ
VMGGENNYSLASHHIFGITGSSQMLFSPLERAQNGMSIYLTDKEKIYEY
IIKDVFTVAPERVDVIDDTAGLKEVTLVTCTDIEATERIIVKGELKTEY
DFDKAPADVLKAFNHSYNQVST

The sequence of SrtB is set forth in SEQ ID NO: 54:

EDKQERANYEKLQQKFQMLMSKHQEHVRPQFESLEKINKDIVGWIKLSG
TSLNYPVLQGKTNHDYLNLDFEREHRRKGSIFMDFRNELKNLNHNTILY
GHHVGDNTMFDVLEDYLKQSFYEKHKIIEFDNKYGKYQLQVFSAYKTTT
KDNYIRTDFENDQDYQQFLDETKRKSVINSDVNVTVKDRIMTLSTCEDA
YSETTKRIVVVAKIIKVS

The sequence of SrtC is set forth in SEQ ID NO: 55:

KTTIQKYTRNVETLEPAQAKHLKEEAALYNQYIYTKSQYQSWNKAVPEY
KKQLITDKDKVIAYLSIPQIKITNIPVYSGDGEETLAAGVGHIPQTSLP
IGGENTHAVLSAHSGHINNTLFSDLEDLKMKDVFYIHVLDQTLKYEIFE
RKIVNPEDTDAINVIPGKDLVTLVTCWPTGINNKRLLVTGRRVATTTMT
PQEHIQRNKYG

The sequence of SrtD is set forth in SEQ ID NO: 56:

KLIDTNTKTEQTLKEAKLAAKKPQEASGTKNSTDQAKNKASFKPETGQA
SGILEIPKINAELPIVEGTDADDLEKGVGHYKDSYYPDENGQIVLSGHR
DTVFRRTGELEKGDQLRLLLSYGEFTYEIVKTKIVDKDDTSIITLQHEK
EELILTTCYPFSYVGNAPKRYIIYGKRVT

The sequence of SrtE1 is set forth in SEQ ID NO: 57:

TNVRAHAQANQAASNLQDDWANGKRSPGSFEPGQGFALLHIPKLDVVVP
IAEGISSKKVLDRGMVGHYAEDGLKTAMPDAKAGNFGLAGHRNTHGEPF
RYINKLEPGDPIVVETQDKYFVYKMASILPVTSPSNVSVLDPVPKQSGF
KGPGRYITLTTCTPEFTSKYRMIVWGKMVEERPRSKGKPDALVS

The sequence of SrtE2 is set forth in SEQ ID NO: 58:

SLWWTNVVADRAADKQAEKVRDDWAQDRVGGSGQDGPGALDTKAGIGFL
HVPAMSEGDILVEKGTSMKILNDGVAGYYTDPVKATLPTSDEKGNFSLA
AHRDGHGARFHNIDKIEKGDPIVFETKDTWYVYKTYAVLPETSKYNVEV
LGGIPKESGKKKAGHYITLTTCTPVYTSRYRYVVWGELVRTEKVDGDRT
PPKELR

The sequence of mgSrtA−ΔN59 is set forth in SEQ ID NO: 64:

QAKPQIPKDKSKVAGYIEIPDADIKEPVYPGPATREQLNRGVSFAEENE
SLDDQNISIAGHTFIGRPNYQFTNLKAAKKGSMVYFKVGNETRKYKMTS
IRNVKPTAVGVLDEQKGKDKQLTLITCDDLNRETGVWETRKILVATEVK

The sequence of mgSrtA-K134A is set forth in SEQ ID NO: 65:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGHTFI
GRPNYQFTNLAAAKKGSMVYFKVGNETRKYKMTSIRNVKPTAVGVLDEQ
KGKDKQLTLITCDDLNRETGVWETRKILVATEVK

The sequence of mgSrtA-mono is set forth in SEQ ID NO: 66:

KPHIDNYLHDKDKDEKIEQYDKNVKEQASKDKKQQAKPQIPKDKSKVAG
YIEIPDADIKEPVYPGPATREQLNRGVSFAEENESLDDQNISIAGHTFI
GRPNYQFTALKAAAKGSMVAFKVGNETRKYKMTSIRNVKPTAVGVLDEQ
KGKDKQLTLITCDDLNRETGVWETRKILVATEVK

The sequence of SrtF is set forth in SEQ ID NO: 67:

AAKKGPVPAGCMKTPKPIVPVKYSIDGMKASAKVLSRGVDETGAAGAPP
KNDPSSMAWFNQGPKIGSDKGNAVLTAHTYHKGGALGNRLYDKNNGIKK
GDIIRLTDKTGQTVCYRYDHDTKVMVKDYNPNSNILYDNNGPAQAAIVI
CWDYVKKTGEFDSRVIFYTYPVA

### Example 4: Cell Labeling

DNA, RNA, or peptide was incubated with 0.5 million cells at the presence of mgSrtA (20 mM) in a 50 uL reaction at 37° C. for 10 mins. Concentrations of DNA, RNA, or peptide in a labeling reaction may vary as needed. An exemplary substrate concentration is 100 nM for DNA and RNA and 20 uM for peptide. Reactions were terminated with 50 mM EDTA.

In an “Oligo-1st” labeling experiment, 0.5 million cells were firstly incubated with oligos at 37° C. for 5 mins, followed by the addition of mgSrtA to 20 uM final concentration and incubated at 37° C. for another 10 mins.

In an “Enzyme-1st” labeling experiment, 0.5 million cells were firstly incubated with 20 uM mgSrtA at 37° C. for 5 mins, followed by the addition of oligos and incubated at 37° C. for another 10 mins.

### Example 5: Flow Cytometry Analysis

Before the flow cytometry analysis, 0.5 million cells were washed twice in 1 mL cold PBS supplemented with 1% BSA. After the wash, the cells were resuspended in 200 uL cold PBS and analyzed on BC CytoFLEX LX.

### Example 6: SMART-Seq Library Preparation

After a cell labeling reaction, the cells were washed with PBS for three times. Five hundred cells were counted for both the labeled sample and the un-labeled control sample for Smart-Seq library preparation.

A Smart-Seq (TAKARA 634889) workflow protocol was followed up until the purification of cDNA amplification. The supernatant from the 1×beads selection was collected for an additional 2×right-sided beads selection. The products were then eluted in 12 uL nuclease-free H₂O.

To generate the final library, 2 uL beads elution was amplified in a 50 uL PCR reaction, including 0.5 uL 10 uM “dT primer,” 0.5 uL 10 uM “P7 Primer,” 22 uL nuclease-free water, and 25 uL NEBNext Ultra II Q5 Master Mix (NEB M0544). Two rounds of PCR reactions were performed.

The 1^{st} round of PCR reaction was performed under the following conditions: 98° C. for 30 s, 10/12 cycles (10 cycles for the labeling sample and 12 cycles for un-labeled control sample) of 98° C. for 10 s, 53° C. for 30 s and 72° C. for 15 s, and a final extension step of 72° C. for 2 mins. A total of five PCR reactions in this round were combined and concentrated with an Amicon Ultra 0.5 ml 30 kDa MWCO centrifugal filter (Millipore UFC5030BK) and purified and size-selected with 1.8×AMPure XP beads (Beckman A63882). The amplification products were eluted in 30 uL nuclease-free H₂O.

In the 2^{nd} round of PCR, 2 uL template from the 1^{st} round of PCR reaction was used in each 50 uL reaction, including 25 uL NEBNext Ultra II Q5 Master Mix(NEB M0544), 0.5 uL 10 uM “P5 Primer,” 0.5 uL 10 uM “P7 Primer,” and 22 uL nuclease-free water. The PCR program was set as the follows: 98° C. for 30 s, 8 cycles of 98° C. for 10 s, 66° C. for 30 s and 72° C. for 20 s, and a final extension step of 72° C. for 2 min. A total of twelve reactions were combined in this round and concentrated with the Amicon Ultra 0.5 ml 30 kDa MWCO centrifugal filter (Millipore UFC5030BK). The products were purified and size-selected with 1.4×AMPure XP beads twice.

dT Primer:
(SEQ ID NO: 37)
5′-CTACACGACGCTCTTCCGATCTatggtgagcaagggcgNNNNNNN
NNNTTTTTTTTTTTTTTTTTTTTTTTTTTTTTTVN-3′
P5 Primer:
(SEQ ID NO: 38)
5′-AATGATACGGCGACCACCGAGATCTACACTCTTTCCCTACACGACG
CTCTTCCG-3′
P7 Primer:
(SEQ ID NO: 39)
5′-CAAGCAGAAGACGGCATACGAGATatatcagtGTGACTGGAGTTCA
GACGTGTGC-3′

### Example 7: Imaging

Cells were collected and washed twice with PBS, then split into aliquots of 0.5 million cells in 50 uL HBSS per tube. The cells were labeled by 100 nM oligonucleotide modified with FITC or TAMRA in the presence of 20 uM mgSrtA at 37° C. for 10 minutes. At the end of incubation, the cells were washed with HBSS twice and then transferred to the Nunc Lab-Tek Chambered Coverglass (Thermo Scientific 155411) at the density of 20,000 cells in 300 uL HBSS per well. Confocal images were taken under the FITC or TAMRA channel, laser power=0.5.

### Example 8: Western Blot

DNA oligos and mgSrtA were mixed and incubated at 37° C. for 30 min. At the end of incubation, the reaction was stopped by adding 1×loading dye, and the samples were denatured at 95° C. for 15 mins. The mixture in the samples was then separated in 4-20% Bis-Tris PAGE (GenScript M00656), and transferred onto nitrocellulose membranes (Merck HATF00010). The membranes were blocked by incubating with 5% BSA in 1×TBST (Sangon Biotech C520009-0500) and incubated 2 hours at RT or overnight at 4° C. with anti-biotin antibody (Abcam ab201341) at 1:500 dilution in 5% BSA TBST. Then, the membranes were washed three times with TBST and incubated 1 hour at RT with HRP-conjugated secondary antibodies (Invitrogen 31430) at 1:5000 dilution in 5% BSA TBST. After washing three times with TBST, the membranes were imaged using SuperSignal West Pico PLUS (Thermo 34580).

### Example 9: Enzyme Digestion and the Addition of GAGs

Cells were incubated with a proteinase or a glycosidase before cell labeling. Enzyme digestion was performed with 0.5 million cells in each 50 uL reaction.

### 1. Enzyme Digestion

A total of 0.5 million cells were counted and treated with a glycosidase or a proteinase at a suitable temperature for 1 hour. In some assays, more than one digestive enzyme (e.g., a heparinase I/II/III combination) was used. At the end of the enzymatic treatment, the cells were pelleted by spinning 3 mins at 500 g and washed twice with 1 mL PBS. The cells were then incubated with 20 uM mgSrtA at 37° C. for 5 mins in HBSS, then followed by the addition of an oligonucleotide to a 100 nM final concentration and incubated at 37° C. for another 10 mins.

### 2. Addition of GAG

A total of 0.5 million cells were incubated with 20 uM mgSrtA in the presence of 300 ng/uL glycosaminoglycan at 37° C. for 5 mins. After the incubation, 100 nM oligos or 20 uM peptides were added to the reaction and incubated for another 10 mins at 37° C.

### Example 10: Studies of Sortase-Mediated Nucleic Acid Reactions

### 1. Roles of Oligonucleotides

We conducted mgSrtA-mediated cell labeling with fluorescence-modified DNA oligo by incubating mgSrtA and DNA oligo for 10 mins at 37° C. Fluorescent signals were observed on the surface of K562 cells under confocal microscopy (FIG. 54A, FIG. 61). The labeled cells were then subjected to quantitative analysis by flow cytometry. We found that almost all cells were positively labeled when 100 nM DNA oligos were applied. The mean fluorescence intensity (MFI) was positively correlated with the oligo concentration and had not reached a plateau when 2000 nM oligo was applied (FIG. 54B-C, FIG. 41). The fluorescent signals of the positively labeled cells were detectable after 120 hours under standard cell culture conditions (FIGS. 45 and 46).

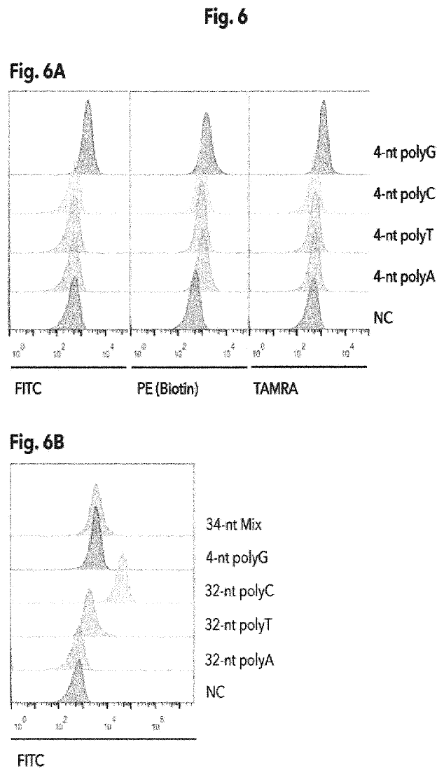

We discovered that mgSrtA facilitated oligonucleotides to be conjugated to cells. To investigate which types of nucleotides that could be more favorably anchored to cell membranes mediated by mgSrtA, we compared the labeling efficiency of four oligonucleotides, each of which contained only one type of the four nucleotides: 4-nt poly G, 4-nt poly C, 4-nt poly A, and 4-nt poly T, each of which was modified with FITC. We labeled K562 cells with the FITC-modified oligos in presence of mgSrtA, with a negative control (NC) without mgSrtA, and quantified the oligo signals using Flow Cytometry (FIG. 6A). The results indicated that the 4-nt guanine oligo (polyG) labeled the most cells and exhibited the highest intensity.

To exclude possible influence from the fluorescent modification group, we repeated the same experiments using biotin-modified and TAMARA-modified 4-nt oligonucleotides. The results indicated that the mgSrtA-dependent cell labeling favored guanine nucleotide (FIG. 6A).

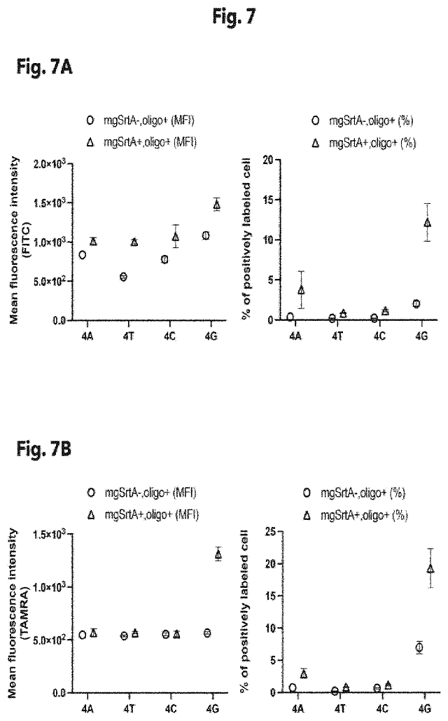

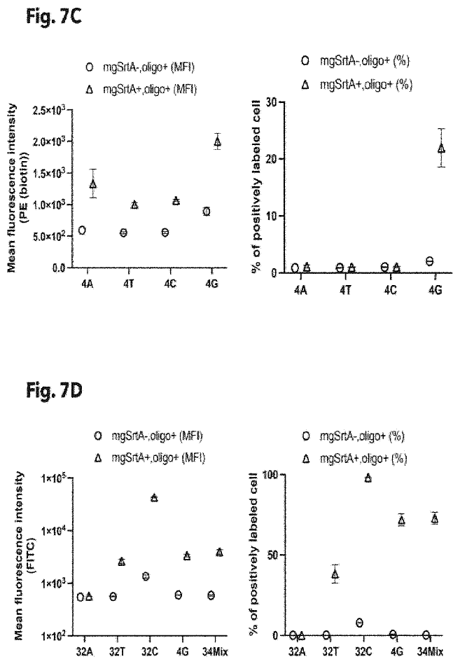

We then increased the number of consecutive nucleotides to 32 nt and found that the 32-nt polyadenine (polyA) was less reactive than other oligos tested (FIG. 6B). The 32-nt poly cytosine (polyC) showed good reactivity when we increased the oligo length. The 32-nt polythymine (polyT) had certain reactivity, but the labeling efficiency was lower than the 4-nt polyG (FIG. 6B). A 32-nt guanine oligo was not included for direct comparison due to limitations of nucleotide synthesis technology. Under each treatment condition, the non-enzyme controls (NC or mgSrtA−) indicated that the labeling reactions were mgSrtA-dependent (FIGS. 6-7). These testing results indicated that the labeling reaction was not non-specific binding between cells and oligonucleotides. Additionally, the distinct activities of polyG, polyC, polyA, and polyT indicated that it may be the nitrogenous base, instead of the carbon sugar or phosphate in the oligonucleotides, mainly contributed to the mgSrtA-mediated oligonucleotide labeling reaction.

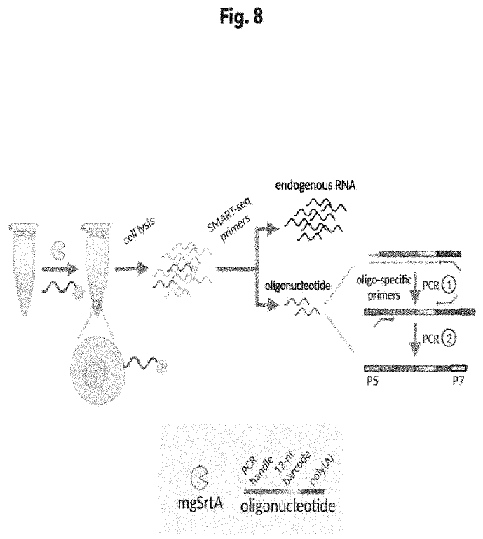

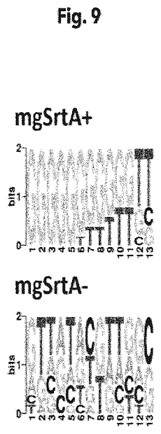

We further investigated nucleotide preferences through a library screen assay. The library included oligonucleotides composed of a 12-nt random sequence (12-nt barcode) for analyzing the nucleotide preferences of mgSrtA. We also included a PCR handle and a polyA sequence surrounding the random sequence to incorporate the SMART-seq library preparation strategy (FIG. 8). We incubated the oligonucleotide library with K562 cells with (mgSrtA+) or without (mgSrtA−) mgSrtA. The oligonucleotides that successfully labeled the K562 cells were enriched and analyzed by high throughput sequencing (HTS). In the sample of mgSrtA+, guanine was overwhelmingly dominant in the 12-nt random sequence region, especially in the first 11 nucleotide positions, while the nucleotides distribution was diverse across the 12-nt random sequence region in the sample of mgSrtA− (FIG. 9). The results (FIGS. 8-9) by library screen and HTS were aligned well with the examination results through the flow cytometry analysis (FIGS. 6-7). These results indicated that G-enriched oligos were preferred by mgSrtA.

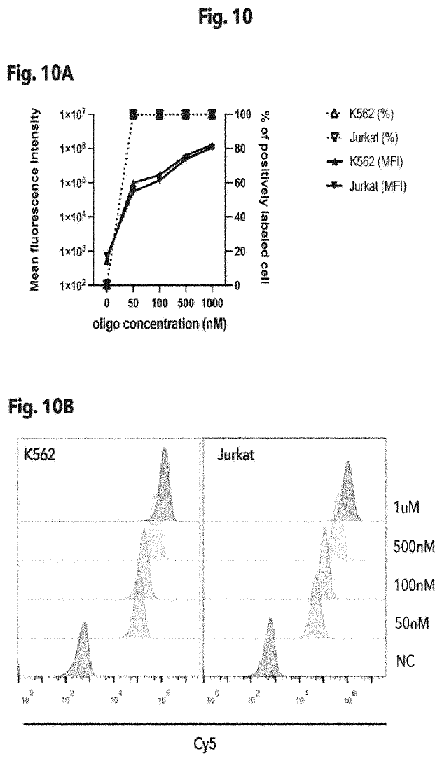

To investigate whether the nitrogenous bases are important in the mgSrtA-dependent cell labeling, we investigated RNA oligos in cell labeling experiments. We performed cell labeling experiments in K562 cells using Cy5-modified RNA oligo at different concentrations. The results show that the RNA oligo also successfully labeled cells in an mgSrtA-dependent manner and the labeling efficiencies are positively correlated with the RNA oligo concentrations (FIG. 10). We repeated the experiment in Jurkat cells and the labeling efficiencies follow similar patterns (FIG. 10).

To further investigate the involvement of nitrogenous base, we performed mgSrtA-dependent cell labeling using dsDNA, in which nitrogenous bases were paired and not readily exposed for reaction. We compared the labeling efficiencies of different sequence configurations, including single-stranded DNA oligo (ssDNA), double-stranded DNA (dsDNA), and partial double-stranded partial single-stranded DNA. We prepared a 45-nt oligonucleotide with 3′-FITC modification (referred as 45*, in which the * indicated fluorescence modification, SEQ ID NO: 4).

Another oligonucleotide with different sequence length and different complementary length were pre-mixed with the 45* DNA at 1:1 molar ratio. We included a 45-nt oligo (denoted as “45”), a 45-nt reverse complementary oligo (denoted as “45RC”), a 30-nt oligo (denoted as “30”), and a 30-nt reverse complementary oligo (denoted as “30RC”). The molarity of the fluorescence modified oligonucleotide across these samples were the same.

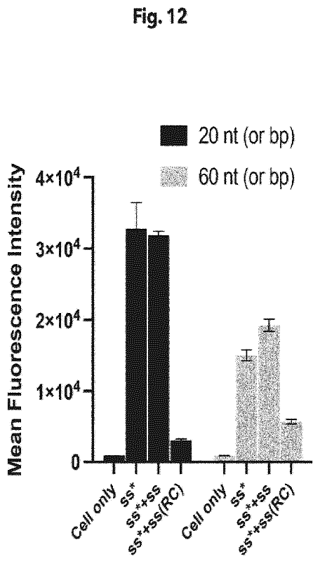

The cells incubated with the various oligos then underwent flow cytometry analysis, and the fluorescence was quantified to represent the labeling efficiencies of these different forms of sequences. The double-stranded form (45*+45RC) labeled cells much less efficiently compared to equal moles of single-stranded form 45*, with the mean fluorescence intensity decreased by 76.7% (FIG. 11). The same experiments were conducted on different sequence lengths of oligos (20 nt (SEQ ID NO: 8) and 60 nt (SEQ ID NO: 10)), and the results were consistent (FIG. 12).

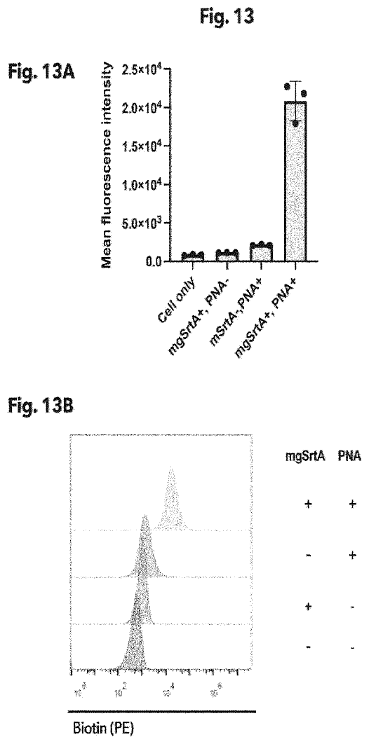

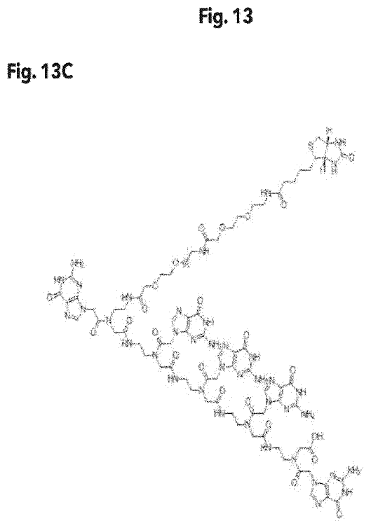

We also examined the labeling efficiency of PNA. A biotinylated PNA was used to label K562 cells. And the results indicated that with the presence of mgSrtA, cells could be efficiently labeled by PNA (FIG. 13). The PNA was ordered from NingBo Karebay Biochem, and the sequence was listed in FIG. 13C.

### 2. Cells Labeled by Oligonucleotides

We investigated the location of the anchored oligos on cells by imaging the labeled cells under confocal microscopy. Similar to the canonical transpeptidation in gram-positive bacteria, we found that the fluorescence signals of fluorescently labeled oligos were located on the cell membranes. These observations were consistent when assayed with different fluorescently-modified oligos and examined in different cell lines (FIGS. 14-16). Although the fluorescent signals were spread on the membranes, the distributions of the signals were aggregated, suggesting the oligos were not evenly distributed on the cell membranes.

### 3. Conjugates of Sortase and Oligonucleotides

mgSrtA Binds with Oligonucleotides

We demonstrated the intermediate products between mgSrtA and varieties of oligonucleotides in vitro. We conducted western blots to analyze the intermediate products of mgSrtA and two biotin-modified oligos (o1 (SEQ ID NO: 15) and o2 (SEQ ID NO: 16) in a cell-free condition (FIG. 17). The product bands also corresponded to the different length of the two oligonucleotides.

More specifically, to further dissect the mgSrtA-mediated cell labeling, we first examined whether mgSrtA binds oligonucleotide in vitro. We incubated biotin-modified 4-mer DNA oligos with mgSrtA and observed shifted bands in western blot (WB) (FIG. 55A). Consistent with the nucleotide preference of mgSrtA in cell labeling, the 4G oligo yielded stronger WB bands than the 4A, 4T, and 4C oligos. And applying a series of guanine oligos (4G, 6G, 8G, 15G, and 20G) generated bands with continuously increased size, which aligned with the increased length of the guanine oligos (FIG. 55B). These results indicated that mgSrtA could bind oligonucleotide in vitro and was independent of cell labeling.

We further investigated whether DNA oligo was covalently bound to mgSrtA. As mgSrtA should have been denatured in WB, the product bands with expected sizes would be present only when the mgSrtA is covalently bound with the 4G oligo. However, it is still possible that the bands resulted from a strong affinity between the 4G oligo and the incompletely denatured mgSrtA, even in a 2% SDS buffer. To rule out the possibility of an affinity-dependent product, we pre-treated mgSrtA in 2% SDS at 95° C. for 10 mins, the same as the sample preparation procedure of western blot. No product band was observed when the 4G oligo was incubated with the pre-treated mgSrtA (FIG. 55C). Based on the testing results, the reaction between the mgSrtA and DNA oligo appear to be covalent and non-covalent interactions may also contribute to the binding between the mgSrtA and DNA oligo.

The canonical function of sortase A is transpeptidase, by which bacteria proteins with LPXTG sorting motifs are cleaved between the thyronine and the glycine and displayed on the cell wall. To test whether the reaction between the mgSrtA and DNA oligo is related to the intrinsic transpeptidase activity, we introduced residues critical to the transpeptidation of wild-type sortase A ²⁵. These mgSrtA mutants (H120A, C184A, R197A and H120A+C184A+R197A) retained activity to react with the 4G oligo, but lost activity with the AALPETG (SEQ ID NO: 19) peptide, which is the substrate in the sortase-catalyzed transpeptidation (FIG. 55D-E). We also examined activities of other mgSrtA mutants. The mgSrtA-mono (N132A+K137A+Y143A), which carries mutations that abolished the dimerization activity of sortase A ²⁶, also yielded products with the 4G oligo but not with the AALPETG (SEQ ID NO: 23) peptide (FIG. 66).

We also screened multiple cations to see if any of them may strengthen the reaction between the mgSrtA and DNA oligo. We added 100 uM various metal cations into the in vitro reaction of the mgSrtA and the 4G oligo. The addition of Cu²⁺ primarily increased the amount of the product compared to no-cation control and other cations (FIG. 55F), suggesting the reaction between the mgSrtA and oligonucleotide could be enhanced by the cation Cu²⁺. Collectively, these lines of evidence support that the mgSrtA binds with oligonucleotides (with a preference for G), which appears to be a covalent binding and distinct from the formation of a thioacylenzyme intermediate in the transpeptidation reaction catalyzed by sortase A.

mgSrtA Bridges Oligonucleotide on the Cell Surface

After having identified the binding between oligonucleotide and mgSrtA, we next investigated how oligonucleotide was labeled to mammalian cell surface mediated by mgSrtA. We observed the mgSrtA, the labeled oligonucleotide, and the cells under confocal microscopy and found that the mgSrtA co-localized with oligonucleotides on the surface of the labeled cells (FIG. 56A). This is an interesting finding as it indicates that the mgSrtA itself is involved in the attachment of oligonucleotide on mammalian cell surface. Additionally, the merged image showed that the fluorescence intensity of the mgSrtA and oligonucleotide were correlated (FIG. 67).

We used flow cytometry to quantify the signals of labeled oligonucleotide and anchored mgSrtA, as well as the mgSrtA mutants known to bind with oligonucleotide in Western Blotting (FIG. 55D, FIG. 66A). Interestingly, the signals of anchored sortase were positively correlated with the corresponding signals of labeled oligonucleotide, which confirmed the participation of mgSrtA as part of the labeled molecules on the cell surface (FIG. 56B-C). However, LPXTG peptide was labeled to cell surface mediated by mgSrtA but not by mgSrtA-triple, mgSrtA-R197A, mgSrtA-C184A, and mgSrtA-H120A, which is probably because there are no bindings between the LPXTG peptide and these mgSrtA mutants (FIG. 68). The oligonucleotide signal on cell surface appears to be mgSrtA-dependent and that mgSrtA is required as part of the labeled moiety.

### Oligonucleotide Binding is a Previously Unknown Property of Wild-Type Sortase

mgSrtA was engineered from the wild-type sortase A, to allow more expansive substrates for transpeptidation. We determined whether the ability to bind oligonucleotide and mediate oligonucleotide cell labeling is previously unrevealed properties of the wild-type sortase A or emerged with the protein engineering of the sortase. First, we expressed and purified wild-type sortase A and three engineered sortase A (5M ⁶, mgSrtA-L200F ⁷, and mgSrtA ⁸). The 5M was named after five mutated residues (P94R, D160N, D165A, K190E, and K196T) in the WT sortase A, the mgSrtA-L200F mutated three further residues (D124G, Y187L, and E189R), and the mgSrtA carries an additional F200L mutation.

Strikingly, both the WT and the engineered sortase A bind to oligonucleotide (FIG. 57A), supporting that binding to oligonucleotide is a previously unrevealed property of the WT sortase A. And the binding between the WT sortase A and oligonucleotide could also be enhanced by metal ion Cu²⁺, the same to the mgSrtA (FIG. 57B, FIG. 75). Next, we applied both the wild-type and engineered sortase A to label oligonucleotide to cells and examined the signals of oligonucleotide and sortase. Flow cytometry data showed that the levels of the anchored sortase and the labeled oligonucleotide are relatively low for the WT sortase A and 5M compared to the other sortases quantified in this experiment, but higher than the no-sortase control (FIG. 57C-D, FIG. 76). We further expressed and purified WT-F200L, a mutant with F200L directly added into the WT sortase A, and observed both the signals of the WT-F200L and the oligonucleotides in cell labeling. Together, both the in vivo binding and cell labeling evidence suggested that the WT sortase A binds oligonucleotides, which was previously unrecognized by the art, and that mediating the oligonucleotide cell labeling is an emergent property of engineered sortase A resulting from the directed evolution, in which the F200L contributed.

We also used docking simulation to predict the possible binding configurations between oligonucleotide and mgSrtA. The resultant docking model was compared with the crystal structure of wild-type sortase A and LPXTG peptide complex (PDB ID: 2KID). The simulation indicated that a 4-mer poly guanine could bind to a separate active site but in the same binding pocket of peptide (FIG. 60), which allows the oligonucleotide accommodation in mgSrtA.

Gram-Positive Bacteria Labels Oligonucleotide at their Surface

Previous reports have demonstrated that the binding of extracellular DNA on the surface of Staphylococcus aureus (S. aureus) contributes to the formation of biofilm of bacteria, but the mechanism is unclear ^{23, 24}. Given the observation that both the mgSrtA and WT sortase A could bind with DNA oligos, we determined whether the surface sortase A of S. aureus could bind DNA oligos, which may contribute to the formation of biofilm. We incubated the FITC-modified 4G, 4C, 4T, and 4A DNA oligos with S. aureus as we did for the mammalian cells, except no exogenous sortase was added. We used flow cytometry to quantify the signals of S. aureus and found that the 4G oligo exhibited a 3-fold higher signal than the other three DNA oligos (FIG. 58A-B), which is consistent with the pattern of mgSrtA-mediated oligonucleotide labeling on mammalian cells.

To further determine whether surface sortase A contributed to the labeling of DNA oligos, we repeated the DNA oligo labeling on E. coli, a gram-negative bacterium with no surface sortase expression (FIG. 58C-D). Across various DNA oligos, the fluorescence signals detected from E. coli remained at the basal level as the “no DNA oligo” control, while signals detected from S. aureus were at least one magnitude higher except for the 32A oligo. Among the examined DNA oligos, the signals of the 32C oligo were 100-fold more elevated than the “no DNA oligo” control. We also demonstrated that the other gram-positive bacteria could also label oligonucleotide although the signal intensity and percentage of positively labeled bacteria are varied across bacteria, e.g. Bacillus subtilis, Enterococcu, and Lactobacillaceae (FIG. 77). Together, these results demonstrated that many gram-positive bacteria, but not E. coli, could directly label oligonucleotide at their surface.

Since multiple classes of sortase are expressed on bacteria surface, the ability to label oligonucleotide of endogenous sortase encouraged us to explore an expanded list of wild-type sortase that can be employed to enable oligonucleotide labeling in the surface of mammalian cell. We expressed sortase A and B from Streptococcus, sortase C from Lactococcus, sortase D from Bacillus, and sortase E1 and E2 from Streptomyces, which were used to label oligonucleotide to cell surface, and both the signal of oligonucleotide and sortase proteins were quantified by flow cytometry (FIG. 58E-F). Surprisingly, sortase E1 exhibited even stronger ability than mgSrtA when label oligonucleotide to cell surface. Sortase E2 and sortase C both show more than one magnitude higher of signals than no sortase control. Signals of sortase proteins also demonstrated that various wild-type sortase from different bacteria strains share the ability to bind cell surface, in which sortase A from S. aureus showed the weakest binding signal. But mgSrtA appears to have acquired its cell surface binding and heparin binding abilities (FIG. 78) through directed evolution.

### 4. Roles of Components in Cell Membrane

We also investigated the possible components on the cell membrane that were involved in the conjugation reaction with oligonucleotides mediated by mgSrtA. Lipids, proteins and carbohydrates are the three macromolecules composing the mammalian cell membrane. Given that the fluorescence signal of sortase and the labeled oligonucleotides on the cell surface appeared to be aggregated (FIG. 56A, FIG. 67), we focused on proteins and carbohydrates rather than the widely distributed membrane lipids.

To investigate whether proteins or carbohydrates in the cell membrane that might be involved in the bioconjugation with an oligonucleotide mediated by a sortase, we employed various proteinases and deglycosylases to disrupt the protein and/or carbohydrate components on the plasma membrane. Cells were pre-treated with digestion enzymes or enzyme combinations and then followed by oligonucleotide labeling in presence of mgSrtA. All proteinases we tested caused more than 50% decrease in labeling efficiency (FIG. 18). Among the proteinases, trypsin and proteinase K have the broadest range of digestive substrates, and these two proteinases caused more than 75% fluorescence intensity decrease.

We next investigated whether the diverse and abundant glycosylations on proteins in the cell membrane contributed to the oligonucleotide labeling reaction. Most transmembrane proteins in animal cells are glycosylated. We included glycosidases targeting O-linked and N-linked glycans, as well as enzymes specifically targeting glycosaminoglycans, including heparinase I/II/III, chondroitinase ABC, and hyaluronidase (FIGS. 19-25). The results indicated that K562 cells exposed to heparinase digestion showed ˜50% fluorescent signal loss. Some heparinase also impacted the labeling efficiency of Jurkat cells and 293T cells, but to a lesser extent compared to K562 cells. The chondroitinases ABC digestion resulted in similar decrease on labeling efficiency and at a similar range in the above three cell types. We also noticed that the combinatorial use of heparinase I/II/III and chondroitinase ABC dramatically impacted the labeling efficiency, of which only 32.5% fluorescence was retained.

We did not observe labeling efficiency decrease with hyaluronidase digestion, which might be because hyaluronic acid has no protein core and is not sulfated. Similarly, PNGase F, which cleaved the innermost GlcNac and asparagine residues from N-linked glycoproteins, and O-Glycosidase, which targeted the Core 1 and Core 3 O-linked disaccharides from glycoproteins, did not impact the labeling as much as heparinase and chondroitinase. Moreover, the use of the commercial NEB Deglycosidase enzyme mix II, which is composed of five different glycosidases, including PNGase F, O-Glycosidase, α2-3,6,8,9 Neuraminidase A, 31-4 Galactosidase S, and 3-N-acetylhexosaminidase, did not decrease the labeling efficiency much.

Additionally, we compared the digestion efficiencies between cell labelings mediated by wild type (WT) SrtA and mgSrtA, in connection with various enzymes. We found that the WT SrtA had lower labeling efficiencies than mgSrtA across the conditions illustrated in FIG. 26 and FIG. 44.

To confirm the involvement of glycosaminoglycan (GAG) in the SrtA-mediated oligonucleotide labeling on cell membranes, we tested several GAGs to investigate whether they could cause decrease of the cell labeling efficiency by oligos. The addition of heparin, heparan sulfate, and chondroitin sulfate significantly impacted the oligonucleotide labeling of cells, while the addition of polyethylene glycol (PEG) did not decrease the efficiency (FIGS. 27-28). These results were consistent across multiple cell types, including K562, Jurkat, Raji, HEK293T, and Hela cells, which indicated that GAG may be involved in oligonucleotide labeling of cells mediated by sortase.

Moreover, the addition of glucose and glycogen exhibited similar patterns as PEG, which indicated their lack of interference with the reactions mediated by mgSrtA (FIGS. 29-30). In addition, heparan sulfate impacted the efficiency of cell labeling stronger than heparin (FIG. 31). Together, these results indicated that a GAG contributed to the mgSrtA-mediated oligo labeling to cell membrane.

We further investigated whether heparin, heparan sulfate, and/or chondroitin were involved in the mgSrtA-mediated oligonucleotide labeling on cell membranes. We tested BaF3, which is a heparan sulfate-negative cell line, and compared the labeling efficiencies of BaF3 with other cell types. The results indicated that BaF3 show much lower labeling efficiencies compared to the other six cell lines (K562, Jurkat, Raji, 293T, Hela, and MC-38) (FIG. 32). The peptide labeling exhibited similar labeling deficiency in BaF3 but to a lesser extent.

The results discussed above indicated the involvement of glycoprotein in the mgSrtA-mediated oligonucleotide labeling on cell membranes. Next, we investigated whether interruptions on biosynthesis enzymes of heparan and chondroitin and proteoglycan core proteins would impact the conjugation between oligonucleotides and the cell membranes. We generated multiple knockout cell lines, in each of which one biosynthesis enzyme or one core protein was disrupted. We compared the labeling efficiencies between the wild-type cells and these knockout cells and found that the knockout of EXT1 (exostosin 1) decreased the labeling efficiency compared to knocking out of other genes (FIG. 33). These results supported the involvement of GAG in mgSrtA-mediated oligonucleotide cell labeling.

We then applied a whole-genome CRISPR screening experiment to look up critical genes affecting the labeling efficiency (FIG. 56D). We used the Brunello library to knockout genes in K562 cells, which were used for mgSrtA-mediated oligonucleotide cell labeling.

The lentivirus Brunello CRISPR screening library were transduced into the K562 cells with stable Cas9 expression at MOI=0.3. Seventy-two hours post-transduction, 2 μg/mL puromycin was added to eliminate the non-transduced cells. After seven days, the cells were labeled with 100 nM DNA oligo (Cy5- or FITC-modified) or 20 uM peptides (FITC- or biotin-modified) with the presence of 20 uM mgSrtA. The cells were washed three times in DPBS before subjected to cell sorting. Cell with the highest 10% MFI and the lowest 10% MFI (˜0.5 million) were sorted on BD FACAria Fusion. Genomic DNA (gDNA) was extracted from the sorted cells. The gRNA cassette was amplified from the gDNA for NGS library preparation. A parallel starting reference, without cell labeling and cell sorting, was included as control sample for the CRISPR screening.

The transduced K562 cells that fell into the bottom 10% MFI were sorted by FACS (Fluorescence-activated Cell sorting), and sgRNAs counts of these cells were compared with a group of control K562 cells transduced with the same CRISPR library without any further treatment. Among the top ten hits from the CRISPR screening, XYLT2 (xylosyltransferase 2) is known as a xylosyltransferase to initiate the tetrasaccharide linker between glycosaminoglycan and core protein, and B4GALT7 (Beta-1,4-Galactosyltransferase 7) and B3GAT3 (Beta-1,3-Glucuronyltrasferase 3) are two galactosyltransferases responsible for the linker elongation. PAPSS1 (3′-Phosphoadenosine 5′-Phosphosulfate Synthase 1) is one of the two synthases to form PAPS, which is a sulfate donor for GAG sulfation (FIG. 56E). To verify the screening results, we conducted mgSrtA-mediated oligonucleotide and peptide labeling using a B4GALT7 knockout cell line and observed 20% and 80% signal reduction of oligonucleotide and peptide, respectively (FIG. 69).

To further confirm the participation of GAG in the anchoring of mgSrtA on cell surface, we examined whether mgSrtA binds with heparin in vitro and in cellula. We used a biotin-modified heparin in Western Blotting and observed binding products when Cu²⁺ is present (FIGS. 70 and 71). The biotin-modified heparin was also applied in cell labeling mediated by mgSrtA, and was labeled to the cell surface, like oligonucleotide, mediated by mgSrtA (FIG. 72).

The screening for AALPETG (SEQ ID NO: 19) peptide cell labeling also identified B4GALT7 as the top hit, indicating the participation of GAG in mgSrtA-mediated peptide cell labeling (FIG. 73). And we observed the co-localization of the labeled AALPETG (SEQ ID NO: 19) peptide and anchored mgSrtA under confocal microscopy (FIG. 74), suggesting mgSrtA also serve as part of the moiety anchored at cell surface in AALPETG (SEQ ID NO: 19) peptide labeling.

Together, our data indicated that mgSrtA is anchored to cell surface to mediate the oligonucleotide and peptide labeling through glycosaminoglycan, e.g., heparin.

### Example 11: CellID Labeling with Oligonucleotides Mediated by mgSrtA

As noted above, sortase-dependent cell labeling by oligos can be used in many applications. For example, it can be used to establish a sequence identifier for each individual cell. This method of labeling cells with oligonucleotides is referred to as CellID herein. To better serve this purpose, we optimized the oligo sequence for better labeling efficiency and ease of characterization.

As with existing cell labeling approaches (e.g., hashtag)²⁰, a CellID oligo may comprise a PCR handle, a barcode region, and a capture sequence. The PCR handle and capture sequence can facilitate downstream molecular biology treatments for making an NGS (next generation sequencing) library. A CellID oligo may also further comprise an anchoring region, preferably enriched with guanine, to be anchored to a cell membrane. For example, an oligo sequence for CellID labeling preferably comprises a guanine-enriched region for high labeling efficiency, a PCR handle for amplification, a programmable region to distinguish individual cells and a capture sequence for oligo enrichment (e.g., poly(A) or the Capture Sequence from 10× genomics, FIG. 34).

We used 100 nM oligo as a starting point to test the labeling conditions, including reaction buffer types (FIG. 35), temperature (FIG. 36), and pH (FIG. 37, FIG. 79). We tested various conditions that were compatible with cell-based assays and observed that CellID labeling was effectively conducted at 37° C. in PBS or HBSS buffer around pH 6.5-8.0. We also noticed that addition of Ca²⁺ did not affect the labeling efficiencies of the Ca²⁺-dependent or Ca²⁺-independent mgSrtA (FIG. 38). Other commonly used cell culture media were used, with or without FBS, but the efficiencies were lower than that in PBS or HBSS buffers (FIG. 35). The labeling reaction also occurred at a relatively lower temperature, e.g., 4° C. or room temperature (RT), but took longer time (FIG. 36). Additionally, we also quantified the EDTA concentration for terminating the labeling reaction to make the CellID labeling more manageable. The results suggested that the labeling was effectively terminated with 30 mM EDTA, and the termination was more complete for the Ca²⁺ dependent mgSrtA (FIG. 39).

We also titrated oligonucleotide concentrations for optimal labeling efficiency. We applied gradient concentrations ranging from 10 nM to 2 uM in CellID labeling. In the first batch of concentration test, we focused on efficiency comparisons when oligonucleotide or peptide was used, respectively. The results indicated that an 86-nt oligonucleotide was more efficiently labeled to the cell membrane compared to a LPXTG peptide at the same molar concentration (FIG. 40).

Next, we conducted a second batch of concentration test on two different cell types. With the increase of oligonucleotide concentrations, more than 90% of cells were quickly labeled at 50 nM and the mean fluorescence intensity kept increasing even at 2 uM (FIG. 41). Interestingly, if cells were labeled with mgSrtA and oligonucleotide together (Together approach), rather than the cells being incubated with the mgSrtA first (enzyme-1^{st} approach), the percentage of positively labeled cells dropped when the oligo concentration exceeds 500 nM, suggesting inhibitory effects with a high concentration of oligonucleotides (FIG. 42). The flow cytometry results also suggested the inhibition was not due to the decrease of cell viability.

A concentration series experiment was also performed with no-sortase control at each concentration gradient. The results showed that the oligonucleotides did not label cells without mgSrtA. And starting from 50 nM of oligonucleotide, the labeling signal was one order of magnitude higher than the respective no-sortase control and was two orders of magnitude higher than the control when 1 uM oligonucleotide was applied (FIG. 43).

### Example 12: Cell Labeling with Oligonucleotides Mediated by Sortase Variants

We compared the labeling abilities of different sortases, including the wild type sortase and different mutants (FIG. 44). Among the enzymes we tested, the WT sortase, Chen2016 8, and mgSrtA all showed labeling efficiencies, although the extents were varied. Among them, the WT sortase showed relatively weak but detectable labeling efficiencies, which was about 1.5-fold compared to the matched no-sortase control. Both Chen2016 and mgSrtA showed strong signals from the labeled oligonucleotides, which were 9-fold and 59-fold of the matched no-sortase control, respectively.

Cell labeling abilities of additional wild-type sortase and sortase variants were tested: WT sortase A, WT sortase B, WT sortase C, WT sortase D, WT sortase E1, WT sortase E2, and WT sortase F as shown in FIG. 58; WT-mono and WT-F200L as shown in FIG. 57C-D; as well as mgSrtA-H120A, mgSrtA-C184A, mgSrtA-R197A, and mgSrtA-triple as shown in FIG. 56B-C.

### Example 13: Retention and Internalization of Oligonucleotides in Cells

We tested the retention time of labeled oligonucleotides on cell surfaces. We continuously cultured the cells for five days after the initial oligonucleotide labeling and measured the fluorescence at multiple timepoints. A 3′-Cy5-modified oligonucleotide was used to avoid degradation during the course of cell culture. At day 5 (120 h), almost all cells remain labeled by oligonucleotide, which were reflected by the 100% positively labeled cells (FIGS. 45-46). The mean fluorescence intensity dropped at a linear rate along culture time increasing, which was about 4.4% of that at the 4^{th} hour and 14.4% of that at the 24^{th} hour. However, even at the last time point, the mean fluorescence was still more than one order of magnitude higher compared to the no-enzyme control, which was sufficient to distinguish the labeled cells from negative control cells. The high signal-to-noise ratio (e.g., the MFI of cells that were labeled compared to those that were not labeled) was high even at 120 h. This observation indicated that even at latter time point, we could still distinguish the labeled cells from background. This could enable applications where the cell labeling by oligos requires longer time.

To visualize the distribution of oligonucleotides in the cells during the process of cell culture, we also imaged the labeled cells at several time points. Surprisingly, we found that some of the oligos had entered the cells at the time point of 12^{th} hr. And at the latter time points, almost all signals came from inside of the cells (FIG. 47). These observations indicated that the oligonucleotides entered cells in regular culture condition.

We also included a plasmid comprising a GFP sequence in a cell labeling and internalization test. Surprisingly, after 48 hrs, GFP fluorescence was observed inside 293T cells that were labeled with the GFP plasmid in the presence of mgSrtA (FIG. 48). These results indicated that cell labeling by oligos in presence of a sortase can provide a new method to deliver and express a plasmid or other external nucleic acids such as a drug or vaccine either in vitro or in a subject.

### Example 14: Diverse Cell Types for Oligonucleotide Labeling

To expand the applications, we also labeled with an oligonucleotide various types of cell lines including cancer cells and embryonic stem cells, as well as diverse types of primary cells (FIGS. 49-50). The cells tested were derived from diverse origins, including cancer cell lines, stem cells, mice spleen, thymus, kidney, liver, lung, bone marrow, as well as the red blood cell. These cells were efficiently labeled by an oligonucleotide with at least two orders of magnitude signal-to-noise ratio compared to the no-enzyme control. These results demonstrated that cell labeling by oligonucleotides can be applied to a variety range of cell types. For example, labeling by CellID can be used as a universal cell labeling method.

### Example 15: CellID-Enabled Sample Multiplexing for scRNA-Seq

The bioconjugation between oligonucleotide and the plasma membrane of cells can be used to connect cell identity with a nucleotide sequence, which can be easily characterized by a high throughput approach. We evaluated the performance of a CellID application in sample multiplexing of single cell RNA-seq (scRNA-seq). CellID labeling can be applied to multiple cell samples, and the cell samples can be simultaneously analyzed in a single experiment. This will eliminate the batch effects and reduce costs in library preparation of scRNA-seq. For example, we labeled different types of cell with distinct CellID oligonucleotides and mixed them for scRNA-seq on the 10× platform as illustrated in FIG. 51.

More specifically, to demonstrate the multiplexing capability, we used eight different oligos (CellIDs: CA11 to CA18), and each oligo was used to label one cell line (FIG. 52). In total, five types of human cells and three types of mouse cells were labeled by the respective CellID oligos and the labeled cells were then mixed. As these cells are derived from different cell types and represent distinct gene expression profiles, we investigated whether the CellID could echo their cell type classification inferred from single cell transcriptome clustering. We used Seurat to generate clusters for the 10,392 cells that passed 10× standard data processing pipeline and visualized the cells in tSNE plot (FIG. 53). Interestingly, each CellID projected to 1 to 2 clusters, and the projections were mutually exclusive among all CellIDs. We then annotated the tSNE clusters according to the marker gene expression, as well as the CellIDs. The annotations by these two methods greatly matched (FIG. 53), which suggested that the CellIDs echoed the cell type classifications precisely. Together, these results showed the robustness of a CellID method to distinguish samples from different species, as well as samples from different cell origin of the same species. CellID can enable simultaneous analysis of multiplexed samples in scRNA-seq experiments. More experimental details for the sample multiplexing are provided below.

### 1. Sample Preparation

Around 0.5 million cells in each sample were pelleted by centrifuging at 500 g for 3 minutes. The pellets were washed twice with PBS and resuspended in a 50 uL labeling buffer, containing 100 nM oligonucleotide and 20 uM mgSrtA. Cells were incubated in the labeling buffer at 37° C. for 10 minutes and then the labeling reaction was terminated by addition of 50 mM EDTA. Cell were then pelleted at 500 g for 3 min at 4° C. and washed with 1 mL cold PBS for three times. The PBS was supplemented with 1% BSA and 30 mM EDTA in the 1^{st} wash and then 0.04% BSA in the 2^{nd} and the 3^{rd} wash. Cells were resuspended in PBS with 0.04% BSA. Multiple samples were then combined in a desired ratio and subjected for 10× Genomics. During the sample preparation, each tube was pre-rinsed with 1 mL of PBS containing 1% BSA. After each round of wash, the supernatant was transferred to a new pre-rinsed tube.

2. scRNA-Seq Library Preparation

The 10× Genomics Single Cell 3′ v3 workflow protocol was followed until the cDNA amplification step. To amplify the labeling oligo together with the cDNA of the labeled cell, PCR reactions were conducted.

When a labeling oligo that does not comprise the 10× capture sequence at the 3′ end was used (e.g., a labeling oligo comprising a polyA sequence as a capture sequence, referred to as a polyA CellID), 0.5 uL 2 uM “2.0 1st nested PCR primer” was added to the cDNA PCR mix. When a labeling oligo comprising the 10× capture sequence at the 3′ end (referred to as CA CellID) was used, another 0.5 uL of 2 uM “Partial Read1N primer” was added.

2.0 1st nested PCR primer: 5′-CCACTCACATCCACTACCAACACT-3′ (SEQ ID NO: 40).

Partial Read1N primer: 5′-GCAGCGTCAGATGTGTATAAGAGACAG-3′ (SEQ ID NO: 41).

The cDNA amplification productions were size selected with 0.6×AMPure XP beads. The long fragments fraction was subjected to the cDNA library preparation following the manufacturer's instructions, which resulted in the mRNA libraries.

For the supernatant of the 0.6×beads selection, another 1.4×beads were added to enrich the short fragments originated from the labeling oligo. The beads were washed twice with 200 uL 80% ethanol and eluted in 40 uL Buffer EB (Qiagen 1014608). The polyA CellID library was amplified using the “P5 Sample index4 bp primer” and “2.0 P7 Read2 indx2 primer,” and the CA CellID library was amplified using the “P5 Read1N primer” and “2.0 P7 Read2 indx2 primer.” PCR was performed in 50 uL volume including 2.5 uL cDNA, 1.25 uL 10 uM forward primer, 1.25 uL of 10 uM reverse primer, 17.5 uL nuclease-free water, and 25 μL of NEBNext Ultra II Q5 Master Mix (NEB M0544). The PCR reactions were carried out under the following conditions: 98° C. for 30 s, 8˜16 cycles of 98° C. for 10 s, 55° C. (polyA CellID) or 66° C. (CS CellID) for 30 s and 72° C. for 15 s, and a final extension step of 72° C. for 2 mins. The nucleotide libraries were cleaned up with 1.2×SPRI beads. These procedures resulted in the CellID libraries for further analysis.

P5 Sample index4bp primer:
(SEQ ID NO: 42)
5′-AATGATACGGCGACCACCGAGATCTACACTAATCTTAACACTCTTT
CCCTACACGACGCTC-3′.
P5 Read1N primer:
(SEQ ID NO: 43)
5′-AATGATACGGCGACCACCGAGATCTACACTCGTCGGCAGCGTCAGA
TGTGTATAAGAGACAG-3′.
2.0 P7 Read2 indx2 primer:
(SEQ ID NO: 44)
5′-CAAGCAGAAGACGGCATACGAGATCTATCGCTGTGACTGGAGTTCA
GACGTGTGCTCTTCCGATCTTCACATCCACTACCAACACTCT-3′.

### 3. Computational Methods

### Screen

We trimmed adapters from the sequencing data using cutadapt software ²¹, and reads without appropriate adapter was removed. Then the random barcode sequence were extracted from the reads and the nucleotide frequency were summarized.

10× scRNA-seq

The 10× scRNA-seq data was processed using the Cell Ranger Single-Cell Software. The sequencing reads of the mRNA library were aligned to the reference genome with default parameters. The reads from CellID libraries were aligned to their own references. The processed data from the CellID libraries and the mRNA library were combined according to the 10× cell barcode.

### Example 16: Summary of Studies of Cell Labeling by Oligonucleotides Mediated by Sortase

The inventors surprisingly discovered that oligonucleotides were conjugated to cell membranes mediated by a sortase, e.g., mgSrtA, a SrtA mutant reported by the Chen's group 9. The mgSrtA enzyme, as well as its diverse variants, was considered to catalyze a transpeptidation reaction of peptides with a sorting motif (e.g., LPXTG) and a nucleophile substrate (e.g., N-oligoglycine). However, in our studies, both DNA and RNA can be catalyzed by a sortase to anchor to the membrane of a cell. This is the first time, to our knowledge, that highly programmable nucleic acids can be efficiently labeled to a cell membrane.

To improve labeling efficiency, we employed a screen assay and found that guanine is a favored base, compared to other bases, by mgSrtA. We implemented an oligonucleotide design based on this discovery, referred to as CellID, and utilized it in tests under various reaction conditions. The CellID technique can be used to label diverse cell types, e.g., both primary and immortalized, in a short time, such as less than five minutes, with more than two orders of magnitude fluorescence intensity compared to controls without presence of the sortase enzyme. The reaction conditions for efficient cell labeling can occur in regular cell culture and a living organism, at regular temperature, culture media, reaction buffer, and pH, etc. The gentle condition under which the oligo-labeling action occurs can facilitate wide-range applications of the labeling technique in biomedical studies, disease diagnosis, and medical treatments.

We applied enzyme digestions and added various external molecules to identify the moiety associated with the cell membrane that contributed to the conjugation of the oligonucleotides to the cells. Proteinase digestions negatively impacted the oligo labeling efficiencies to different extents. Not wishing to be bound by this theory, since both chondroitin sulfate and heparin/heparan sulfate significantly influenced the labeling efficiencies, we believe the abundant glycosaminoglycan (GAG), especially the heparin/heparan sulfate and chondroitin sulfate, in the cell membrane were involved in the labeling reaction. This explanation was supported by results of the glycosidase digestion and the addition of GAGs.

We also observed that 3′-Cy5-modified oligonucleotides entered cells during the process of cell culturing. Confocal images indicated that some oligos entered cells at 12 hrs and almost all oligos entered cells at latter time points, such as at 120 hrs. This enables an interesting application to deliver nucleic acids or derivatives into cells. For example, a nucleic acid drug or vaccine can be delivered to a subject mediated by a sortase. A nucleic acid anchor can also be conjugated with another treating modality (e.g., a peptide drug) and serve as a vehicle to deliver that modality into cells. Some somatic cells such as lymphocytes can be labeled by a nucleic acid drug or a drug with a nucleic acid anchor in vitro or in vivo. Such labeled somatic cells can be a carrier of the nucleic acid drug or the drug with a nucleic acid anchor, and deliver the drug to the various sites of a subject.

Previous studies reported that heparan sulfate proteoglycans (HSPG) and chondroitin sulfate proteoglycans (CSPG) could be receptors or co-receptors for temporary cell surface attachment to promote internalization for a variety of macromolecules including DNA and virus ²². In our study, we demonstrated the involvement of GAGs in oligo labeling of the cells based on the observation that heparinase and chondroitinase treatment decreased the oligo labeling efficiency, and the addition of heparin, heparan sulfate and chondroitin sulfate also hindered the oligo labeling. The data from flow cytometry analysis further indicated that the internalization of oligonucleotides was affected by HSPG and CSPG.

The barcode of a CellID oligonucleotide remained in a CellID-labeled cell for five days or more. CellID thus can be used as a robust cell labeling method. A higher initial concentration of an oligo or chemical modifications like 2′-OMe or phosphorothioate for labeling a cell may extend the retention time of the oligo in the cell to some extent. Both the sequences and length of the oligos can have a flexible design.

Also, the ease and stable labeling of oligonucleotides on cell membranes allows addition of programmable sequence information to a cell, which can be decoded in a latter step, for example, sequenced by a sequencer. The CellID labeling technique will enable diverse downstream applications in both the biological research and clinical uses.

Besides protein display, data from this study brought up another potential function of sortase, as a bacteria surface protein. It is known that sortase contributed to the formation of biofilm of bacteria, in which the environmental polysaccharides, protein, lipids and nucleic acids were utilized to build an external film to increase bacteria viability, e.g., guard the bacteria from antibiotic treatment ²⁴. The new discovery of sortase-DNA binding from this study suggested a previous unknown possibility that sortase may recruit environmental nucleic acids to contribute to the formation of biofilm.

Further embodiments are illustrated below.

Embodiment 1. A conjugate of a sortase and a nucleic acid or derivative thereof.

Embodiment 2. The conjugate of embodiment 1, wherein the sortase is selected from sortase A, sortase B, sortase C, sortase D, sortase E, sortase F, and variants thereof (e.g., a sortase selected from SEQ ID NOs: 2, 18, 22, 27, 45-58, and 64-67, or a sortase having an amino acid sequence with at least 80%, 85%, 90%, 95%, 96%, 97%, 98%, 99%, or 100% identity to any one of SEQ ID NOs: 2, 18, 22, 27, 45-58, and 64-67).

Embodiment 3. The conjugate of any one of embodiments 1-2, wherein the sortase is SpySrtA, SrtE1, SrtE2, SrtF, SrtD, or mgSrtA, or a variant thereof.

Embodiment 4. A conjugate of a cell and a nucleic acid or derivative thereof via (e.g., bridged by) a sortase (e.g., a sortase is selected from sortase A, sortase B, sortase C, sortase D, sortase E, sortase F, and variants thereof).

Embodiment 5. The conjugate of embodiment 4, wherein the nucleic acid or derivative thereof is conjugated to the plasma membrane of the cell via a sortase.

Embodiment 6. The conjugate of any one of embodiments 4-5, wherein the cell is selected from primary cells and immortalized cells.

Embodiment 7. The conjugate of any one of embodiments 1-6, wherein the nucleic acid or derivative thereof is selected from DNA, RNA, and PNA.

Embodiment 8. The conjugate of any one of embodiments 1-7, wherein the nucleic acid or derivative thereof is single stranded.

Embodiment 9. A nucleic acid or derivative thereof comprising an anchor region, wherein the anchor region is guanine enriched.

Embodiment 10. A nucleic acid or derivative thereof comprising an anchor region, a region for PCR amplification, a barcode region for identification, and a capture sequence for sequence enrichment.

Embodiment 11. The nucleic acid or derivative thereof of embodiment 10, wherein the anchor region is enriched with guanine, and the region for PCR amplification is guanine-depleted, and the capture sequence is a poly A sequence or a capture sequence suitable for high throughput sequencing.

Embodiment 12. The conjugate of any one of embodiments 1-8, wherein the nucleic acid or derivative thereof is the nucleic acid or derivative thereof of any one of embodiments 9-11.

Embodiment 13. A method of preparing a conjugate of a cell and a nucleic acid or derivative thereof, comprising contacting the nucleic acid or derivative thereof, the cell, and a sortase, optionally in presence of Cu²⁺, wherein the nucleic acid or derivative thereof is conjugated to the cell, and wherein the conjugation of the nucleic acid or derivative thereof and the cell is mediated by the sortase.

Embodiment 14. The method of embodiment 13, wherein the cell is selected from primary cells and immortalized cells.

Embodiment 15. The method of any one of embodiments 13-14, wherein the nucleic acid or derivative thereof is conjugated to the plasma membrane of the cell.

Embodiment 16. The method of any one of embodiments 13-15, wherein a glycosaminoglycan associated with the cell membrane is involved in the conjugation.

Embodiment 17. The method of embodiment 16, wherein the glycosaminoglycan is selected from heparin, heparan sulfate, chondroitin sulfate, and dermatan sulfate.

Embodiment 18. The method of any one of embodiments 13-17, wherein the sortase is selected from sortase A, sortase B, sortase C, sortase D, sortase E, sortase F, and variants thereof.

Embodiment 19. The method of any one of embodiments 13-18, wherein the sortase is SpySrtA, SrtE1, SrtE2, SrtF, SrtD, or mgSrtA, or derivative thereof.

Embodiment 20. The method of any one of embodiments 13-19, wherein the nucleic acid or derivative thereof is selected from DNA, RNA, and PNA.

Embodiment 21. The method of any one of embodiments 13-20, wherein the nucleic acid or derivative thereof is single stranded.

Embodiment 22. The method of any one of embodiments 13-21, wherein the nucleic acid or derivative thereof is the nucleic acid or derivative thereof of any one of embodiments 9-11.

Embodiment 23. The method of any one of embodiments 13-22, wherein the conjugation occurs in vitro or in vivo.

Embodiment 24. The method of any one of embodiments 13-23, wherein the cell is contacted with the nucleic acid or derivative thereof first and then contacted with the sortase.

Embodiment 25. The method of any one of embodiments 13-23, wherein the cell is contacted with sortase first and then contacted with the nucleic acid or derivative thereof.

Embodiment 26. The method of any one of embodiments 13-25, wherein the conjugation occurs in vitro in a reaction medium and wherein the nucleic acid or derivative thereof is present in a concentration ranging from about 1 nM to about 10 uM in the reaction medium.

Embodiment 27. The method of embodiment 26, wherein the contacting is carried out at from about 4° C. to about 40° C.

Embodiment 28. The method of any one of embodiments 26-27, wherein the contacting is carried out for about 1 min to 30 min.

Embodiment 29. The method of any one of embodiments 26-28, further comprising terminating the conjugation of the nucleic acid or derivative thereof and the cell after about 1 min to 30 min of the contacting.

Embodiment 30. A method of delivering a nucleic acid or derivative thereof to a cell, comprising providing the nucleic acid or derivative thereof and a sortase to the vicinity of the cell, optionally in presence of Cu²⁺, wherein the nucleic acid or derivative thereof is conjugated to the cell mediated by the sortase and wherein the nucleic acid or derivative thereof is subsequently internalized into the cell.

Embodiment 31. The method of embodiment 30, wherein the method is carried out in vivo or in vitro.

Embodiment 32. The method of any one of embodiment 30-31, wherein the nucleic acid or derivative thereof comprises a drug.

Embodiment 33. The method of any one of embodiments 31-32, wherein the nucleic acid or derivative thereof comprises a vaccine.

Embodiment 34. The method of any one of embodiments 30-33, wherein the sortase is selected from sortase A, sortase B, sortase C, sortase D, sortase E, sortase F, and variants thereof.

Embodiment 35. The method of any one of embodiments 30-34, wherein the sortase is SpySrtA, SrtE1, SrtE2, SrtF, SrtD, or mgSrtA, or derivative thereof.

Embodiment 36. A method of barcoding a cell, comprising:
contacting a nucleic acid or derivative thereof, the cell, and a sortase, optionally in presence of Cu²⁺, wherein the nucleic acid or derivative thereof is conjugated to the cell, wherein the conjugation of the nucleic acid or derivative thereof and the cell is mediated by the sortase, and wherein the nucleic acid or derivative thereof comprises the nucleic acid or derivative thereof of any one of embodiments 9-11; and
identifying the cell by determining the identity of the nucleic acid or derivative conjugated to the cell.

Embodiment 37. The method of embodiment 36, wherein the method is carried out in vivo or in vitro.

Embodiment 38. The method of any one of embodiments 36-37, wherein the cell is selected from primary cells and immortalized cells.

Embodiment 39. The method of any one of embodiments 36-38, wherein the sortase is selected from sortase A, sortase B, sortase C, sortase D, sortase E, sortase F, and variants thereof.

Embodiment 40. The method of any one of embodiments 36-39, wherein the sortase is SpySrtA, SrtE1, SrtE2, SrtF, SrtD, or mgSrtA, or derivative thereof.

Embodiment 41. The method of any one of embodiments 36-40, wherein the identity of the nucleic acid or derivative conjugated to the cell is determined by high throughput sequencing.

Embodiment 42. A kit comprising a sortase and a nucleic acid or derivative thereof.

Embodiment 43. The kit of embodiment 42, wherein the nucleic acid or derivative thereof is the nucleic acid or derivative thereof of any one of embodiments 9-11.

Embodiment 44. A conjugate of glycosaminoglycan, e.g., heparin, and a sortase.

Embodiment 45. The conjugate of Embodiment 44, wherein the sortase is selected from WT sortase A, WT sortase B, WT sortase C, WT sortase D, WT sortase E, WT sortase F, and variants thereof.

Embodiment 46. The conjugate of any one of Embodiments 44-45, wherein the sortase is Spyra, SrtE1, SrtE2, SrtF, SrtD, or mgSrtA or a variant thereof.

While the disclosure has been particularly shown and described with reference to specific embodiments, it should be understood by those having skill in the art that various changes in form and detail may be made therein without departing from the spirit and scope of the present disclosure as disclosed herein.

### REFERENCES

1. Jacobitz, A. W., Kattke, M. D., Wereszczynski, J. & Clubb, R. T. Sortase Transpeptidases: Structural Biology and Catalytic Mechanism. Adv Protein Chem Struct Biol 109, 223-264 (2017).
2. Pishesha, N., Ingram, J. R. & Ploegh, H. L. Sortase A: A Model for Transpeptidation and Its Biological Applications. Annu Rev Cell Dev Biol 34, 163-188 (2018).
3. Mazmanian, S. K., Liu, G., Jensen, E. R., Lenoy, E. & Schneewind, O. Staphylococcus aureus sortase mutants defective in the display of surface proteins and in the pathogenesis of animal infections. Proc Natl Acad Sci USA 97, 5510-5515 (2000).
4. Samantaray, S., Marathe, U., Dasgupta, S., Nandicoori, V. K. & Roy, R. P. Peptide-sugar ligation catalyzed by transpeptidase sortase: a facile approach to neoglycoconjugate synthesis. J Am Chem Soc 130, 2132-2133 (2008).
5. Bellucci, J. J., Bhattacharyya, J. & Chilkoti, A. A noncanonical function of sortase enables site-specific conjugation of small molecules to lysine residues in proteins. Angew Chem Int Ed Engl 54, 441-445 (2015).
6. Glasgow, J. E., Salit, M. L. & Cochran, J. R. In Vivo Site-Specific Protein Tagging with Diverse Amines Using an Engineered Sortase Variant. J Am Chem Soc 138, 7496-7499 (2016).
7. Chen, I., Dorr, B. M. & Liu, D. R. A general strategy for the evolution of bond-forming enzymes using yeast display. Proc Natl Acad Sci USA 108, 11399-11404 (2011).
8. Chen, L. et al. Improved variants of SrtA for site-specific conjugation on antibodies and proteins with high efficiency. Sci Rep 6, 31899 (2016).
9. Ge, Y. et al. Enzyme-Mediated Intercellular Proximity Labeling for Detecting Cell-Cell Interactions. J Am Chem Soc 141, 1833-1837 (2019).
10. Podracky, C. J. et al. Laboratory evolution of a sortase enzyme that modifies amyloid-beta protein. Nat Chem Biol 17, 317-325 (2021).
11. Bradshaw, W. J. et al. Molecular features of the sortase enzyme family. FEBS J 282, 2097-2114 (2015).
12. Li, Q., Ren, J., Liu, W., Jiang, G. & Hu, R. CpG Oligodeoxynucleotide Developed to Activate Primate Immune Responses Promotes Antitumoral Effects in Combination with a Neoantigen-Based mRNA Cancer Vaccine. Drug Des Devel Ther 15, 3953-3963 (2021).
13. Juliano, R. L. The delivery of therapeutic oligonucleotides. Nucleic Acids Res 44, 6518-6548 (2016).
14. Yang, H., Wang, H., Ren, J., Chen, Q. & Chen, Z. J. cGAS is essential for cellular senescence. Proc Nat Acad Sci USA 114, E4612-E4620 (2017).
15. Kell, A. M. & Gale, M., Jr. RIG-I in RNA virus recognition. Virology 479-480, 110-121 (2015).
16. Roberts, T. C., Langer, R. & Wood, M. J. A. Advances in oligonucleotide drug delivery. Nat Rev Drug Discov 19, 673-694 (2020).
17. Kole, R., Krainer, A. R. & Altman, S. RNA therapeutics: beyond RNA interference and antisense oligonucleotides. Nat Rev Drug Discov 11, 125-140 (2012).
18. Pardi, N., Hogan, M. J., Porter, F. W. & Weissman, D. mRNA vaccines—a new era in vaccinology. Nat Rev Drug Discov 17, 261-279 (2018).
19. Shi, J. et al. Engineered red blood cells as carriers for systemic delivery of a wide array of functional probes. Proc Natl Acad Sci USA 111, 10131-10136 (2014).
20. Stoeckius, M. et al. Cell Hashing with barcoded antibodies enables multiplexing and doublet detection for single cell genomics. Genome Biol 19, 224 (2018).
21. Martin, M. Cutadapt Removes Adapter Sequences From High-Throughput Sequencing Reads. EMBnet 17 (2011).
22. Park, H. et al. Heparan sulfate proteoglycans (HSPGs) and chondroitin sulfate proteoglycans (CSPGs) function as endocytic receptors for an internalizing anti-nucleic acid antibody. Sci Rep 7, 14373 (2017).

1. A conjugate of a sortase and a nucleic acid or derivative thereof.

2. The conjugate of claim 1, wherein the sortase is selected from WT sortase A, WT sortase B, WT sortase C, WT sortase D, WT sortase E, WT sortase F, and variants thereof.

3. The conjugate of claim 1, wherein the sortase is SpySrtA, SrtE1, SrtE2, SrtF, SrtD, or mgSrtA or a variant thereof.

4. A conjugate of a cell and a nucleic acid or derivative thereof via a sortase.

5. The conjugate of claim 4, wherein the nucleic acid or derivative thereof is conjugated to the plasma membrane of the cell via a sortase.

6. The conjugate of claim 4, wherein the cell is selected from primary cells and immortalized cells.

7. The conjugate of claim 4, wherein the nucleic acid or derivative thereof is selected from DNA, RNA, and PNA.

8. The conjugate of claim 4, wherein the nucleic acid or derivative thereof is single stranded.

9. A nucleic acid or derivative thereof comprising an anchor region, wherein the anchor region is guanine enriched.

10. The nucleic acid or derivative thereof of claim 9, further comprising a region for PCR amplification, a barcode region for identification, and a capture sequence for sequence enrichment.

11. The nucleic acid or derivative thereof of claim 10, wherein the anchor region is enriched with guanine, and the region for PCR amplification is guanine-depleted, and the capture sequence is a poly A sequence or a capture sequence suitable for high throughput sequencing.

12. The conjugate of claim 4, wherein the nucleic acid or derivative thereof is the nucleic acid or derivative thereof of claim 11.

13. A method of preparing a conjugate of a cell and a nucleic acid or derivative thereof, comprising contacting the nucleic acid or derivative thereof, the cell, and a sortase, optionally in presence of Cu²⁺, wherein the nucleic acid or derivative thereof is conjugated to the cell, and wherein the conjugation of the nucleic acid or derivative thereof and the cell is mediated by the sortase.

14. The method of claim 13, wherein the cell is selected from primary cells and immortalized cells.

15. The method of claim 13, wherein the nucleic acid or derivative thereof is conjugated to the plasma membrane of the cell via the sortase.

16. The method of claim 13, wherein a glycosaminoglycan associated with the cell membrane is involved in the conjugation.

17. The method of claim 16, wherein the glycosaminoglycan is selected from heparin, heparan sulfate, chondroitin sulfate, and dermatan sulfate.

18. The method of claim 13, wherein the sortase is selected from sortase A, sortase B, sortase C, sortase D, sortase E, sortase F, and variants thereof.

19. The method of claim 13, wherein the sortase is SpySrtA, SrtE1, SrtE2, SrtF, SrtD, or mgSrtA, or a variant thereof.

20. The method of claim 13, wherein the nucleic acid or derivative thereof is selected from DNA, RNA, and PNA.

21. The method of claim 13, wherein the nucleic acid or derivative thereof is single stranded.

22. The method of claim 13, wherein the nucleic acid or derivative thereof is the nucleic acid or derivative thereof of claim 11.

23. The method of claim 13, wherein the conjugation occurs in vitro or in vivo.

24. The method of claim 13, wherein the cell is contacted with the nucleic acid or derivative thereof first and then contacted with the sortase.

25. The method of claim 13, wherein the cell is contacted with sortase first and then contacted with the nucleic acid or derivative thereof.

26. The method of claim 13, wherein the conjugation occurs in vitro in a reaction medium and wherein the nucleic acid or derivative thereof is present in a concentration ranging from about 1 nM to about 10 uM in the reaction medium.

27. The method of claim 26, wherein the contacting is carried out at from about 4° C. to about 40° C.

28. The method of claim 26, wherein the contacting is carried out for about 1 min to 30 min.

29. The method of claim 26, further comprising terminating the conjugation of the nucleic acid or derivative thereof and the cell after about 1 min to 30 min of the contacting.

30. A method of delivering a nucleic acid or derivative thereof to a cell, comprising providing the nucleic acid or derivative thereof and a sortase to the vicinity of the cell, optionally in presence of Cu²⁺, wherein the nucleic acid or derivative thereof is conjugated to the cell mediated by the sortase and wherein the nucleic acid or derivative thereof is subsequently internalized into the cell.

31. The method of claim 30, wherein the method is carried out in vivo or in vitro.

32. The method of claim 30, wherein the nucleic acid or derivative thereof comprises a drug.

33. The method of claim 30, wherein the nucleic acid or derivative thereof comprises a vaccine.

34. The method of claim 30, wherein the sortase is selected from sortase A, sortase B, sortase C, sortase D, sortase E, sortase F, and variants thereof.

35. The method of claim 30, wherein the sortase is SpySrtA, SrtE1, SrtE2, SrtF, SrtD, or mgSrtA, or variant thereof.

36. A method of barcoding a cell, comprising:
contacting a nucleic acid or derivative thereof, the cell, and a sortase, optionally in presence of Cu²⁺, wherein the nucleic acid or derivative thereof is conjugated to the cell, wherein the conjugation of the nucleic acid or derivative thereof and the cell is mediated by the sortase, and wherein the nucleic acid or derivative thereof comprises the nucleic acid or derivative thereof of claim 11; and
identifying the cell by determining the identity of the nucleic acid or derivative conjugated to the cell.

37. The method of claim 36, wherein the method is carried out in vivo or in vitro.

38. The method of claim 36, wherein the cell is selected from primary cells and immortalized cells.

39. The method of claim 36, wherein the sortase is selected from sortase A, sortase B, sortase C, sortase D, sortase E, sortase F, and variants thereof.

40. The method of claim 36, wherein the sortase is SpySrtA, SrtE1, SrtE2, SrtF, SrtD, or mgSrtA, or variant.

41. The method of claim 36, wherein the identity of the nucleic acid or derivative conjugated to the cell is determined by high throughput sequencing.

42. A kit comprising a sortase and a nucleic acid or derivative thereof of claim 9.

43. The kit of claim 42, wherein the nucleic acid or derivative thereof is the nucleic acid or derivative thereof of claim 11.

44. A conjugate of glycosaminoglycan and a sortase.


SEQUENCE LISTING (69 sequences)

SEQ ID NO: 1 - DNA, 34 bp, synthetic construct
  misc_feature 1..34: note = Artificial Sequence
  source 1..34: mol_type = other DNA
gtgactggagttcagacgtgtgctcttccgatct

SEQ ID NO: 2 - protein, 181 aa, synthetic construct
  REGION 1..181: note = mgSrtA
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 3 - RNA, 50 nt, synthetic construct
  misc_feature 1..50: note = Artificial Sequence
  source 1..50: mol_type = other RNA
ggggtggggcggggaaacacatccactaccaacactctgctttaaggccg

SEQ ID NO: 4 - DNA, 45 bp, synthetic construct
  misc_feature 1..45: note = Artificial Sequence
  source 1..45: mol_type = other DNA
atcgatcgatgctagctagcgttcagacgtgtgctcttccgatct

SEQ ID NO: 5 - DNA, 30 bp, synthetic construct
  misc_feature 1..30: note = Artificial Sequence
  source 1..30: mol_type = other DNA
acgtctgaacgctagctagcatcgatcgat

SEQ ID NO: 6 - DNA, 30 bp, synthetic construct
  misc_feature 1..30: note = Artificial Sequence
  source 1..30: mol_type = other DNA
atcgatcgatgctagctagcgttcagacgt

SEQ ID NO: 7 - DNA, 45 bp, synthetic construct
  misc_feature 1..45: note = Artificial Sequence
  source 1..45: mol_type = other DNA
agatcggaagagcacacgtctgaacgctagctagcatcgatcgat

SEQ ID NO: 8 - DNA, 20 bp, synthetic construct
  misc_feature 1..20: note = Artificial Sequence
  source 1..20: mol_type = other DNA
atcgatcgatgctagctagc

SEQ ID NO: 9 - DNA, 20 bp, synthetic construct
  misc_feature 1..20: note = Artificial Sequence
  source 1..20: mol_type = other DNA
gctagctagcatcgatcgat

SEQ ID NO: 10 - DNA, 60 bp, synthetic construct
  misc_feature 1..60: note = Artificial Sequence
  source 1..60: mol_type = other DNA
atcgatcgatgctagctagcgttcagacgtgtgctcttccgatctgtgactggagttcag

SEQ ID NO: 11 - DNA, 60 bp, synthetic construct
  misc_feature 1..60: note = Artificial Sequence
  source 1..60: mol_type = other DNA
ctgaactccagtcacagatcggaagagcacacgtctgaacgctagctagcatcgatcgat

SEQ ID NO: 12 - DNA, 100 bp, synthetic construct
  misc_feature 1..100: note = Artificial Sequence
  source 1..100: mol_type = other DNA
ggggcggggtggggcggggaaatcatctcaaccactcacatccactaccaacactcthhactcacttaahhhhhbaaaaaaaaaaaaaaaaaaaaaaaaa

SEQ ID NO: 13 - DNA, 100 bp, synthetic construct
  misc_feature 1..100: note = Artificial Sequence
  source 1..100: mol_type = other DNA
ggggcggggtggggcggggaaatcatctcaaccactcacatccactaccaacactcthhaacatatctchhhhhbaaaaaaaaaaaaaaaaaaaaaaaaa

SEQ ID NO: 14 - DNA, 100 bp, synthetic construct; identical to SEQ ID NO: 13
  misc_feature 1..100: note = Artificial Sequence
  source 1..100: mol_type = other DNA

SEQ ID NO: 15 - skipped

SEQ ID NO: 16 - DNA, 20 bp, synthetic construct
  misc_feature 1..20: note = Artificial Sequence
  source 1..20: mol_type = other DNA
ggggcggggtggggcggggt

SEQ ID NO: 17 - DNA, 79 bp, synthetic construct
  misc_feature 1..79: note = Artificial Sequence
  misc_feature 35..46: note = n is a, c, g, or t
  source 1..79: mol_type = other DNA
gtgactggagttcagacgtgtgctcttccgatctnnnnnnnnnnnnbaaaaaaaaaaaaaaaaaaaaaaaaaaaaaaaa

SEQ ID NO: 18 - protein, 181 aa, Staphylococcus aureus
KPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 19 - protein, 7 aa, synthetic construct
  REGION 1..7: note = Artificial Sequence
AALPETG

SEQ ID NO: 20 - DNA, 12 bp, synthetic construct
  misc_feature 1..12: note = Artificial Sequence
  source 1..12: mol_type = other DNA
gctttaaggccg

SEQ ID NO: 21 - DNA, 14 bp, synthetic construct
  misc_feature 1..14: note = Artificial Sequence
  source 1..14: mol_type = other DNA
gctcacctattagc

SEQ ID NO: 22 - protein, 181 aa, synthetic construct
  REGION 1..181: note = Artificial Sequence
KPHIDNYLHDKDKDEKIEQYDKNVKEQASKDKKQQAKPQIPKDKSKVAGYIEIPDADIKEPVYPGPATREQLNRGVSFAKENQSLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 23 - protein, 7 aa, synthetic construct
  REGION 1..7: note = Artificial Sequence
AALPETG

SEQ ID NO: 24 - DNA, 86 bp, synthetic construct
  misc_feature 1..86: note = Artificial Sequence
  source 1..86: mol_type = other DNA
ggggcggggtggggcggggaaatcatctcaaccactcacatccactaccaacactcthhcatcatcaathhhhhgctttaaggccg

SEQ ID NO: 25 - DNA, 95 bp, synthetic construct
  misc_feature 1..95: note = Artificial Sequence
  source 1..95: mol_type = other DNA
ggggcggggtggggaaagtgactggagttcagacgtgtgctcttccgatcthhhaagttcaagttagachhhagctttaaggccggtcctagcaa

SEQ ID NO: 26 - protein, 147 aa, synthetic construct
  REGION 1..147: note = Artificial Sequence
QAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVK

SEQ ID NO: 27 - protein, 181 aa, synthetic construct
  REGION 1..181: note = Artificial Sequence
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKIFVATEVK

SEQ ID NO: 28 - DNA, 3713 bp, synthetic construct
  misc_feature 1..3713: note = Artificial Sequence
  source 1..3713: mol_type = other DNA
caactttgtatagaaaagttgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgcctcgagggtacttatataagggggtgggggcgcgttcgtcctcagtcgcgatcgaacactcgagccgagcagacgtgcctacggaccgtctagacaagtttgtacaaaaaagcaggctgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgacgcaccaaggaagccctcgaggacgcgtaaaggtaccaaaggatcccgacctaccgacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

SEQ ID NO: 29 - DNA, 100 bp, synthetic construct
  misc_feature 1..100: note = Artificial Sequence
  source 1..100: mol_type = other DNA
ggggcggggtggggcggggaaatcatctcaaccactcacatccactaccaacactcthhcatatcactahhhhhbaaaaaaaaaaaaaaaaaaaaaaaaa

SEQ ID NO: 30 - DNA, 100 bp, synthetic construct
  misc_feature 1..100: note = Artificial Sequence
  source 1..100: mol_type = other DNA
ggggcggggtggggcggggaaatcatctcaaccactcacatccactaccaacactcthhcatcatcaathhhhhbaaaaaaaaaaaaaaaaaaaaaaaaa

SEQ ID NO: 31 - DNA, 100 bp, synthetic construct
  misc_feature 1..100: note = Artificial Sequence
  source 1..100: mol_type = other DNA
ggggcggggtggggcggggaaatcatctcaaccactcacatccactaccaacactcthhtatacaccathhhhhbaaaaaaaaaaaaaaaaaaaaaaaaa

SEQ ID NO: 32 - DNA, 100 bp, synthetic construct
  misc_feature 1..100: note = Artificial Sequence
  source 1..100: mol_type = other DNA
ggggcggggtggggcggggaaatcatctcaaccactcacatccactaccaacactcthhacattactachhhhhbaaaaaaaaaaaaaaaaaaaaaaaaa

SEQ ID NO: 33 - DNA, 100 bp, synthetic construct
  misc_feature 1..100: note = Artificial Sequence
  source 1..100: mol_type = other DNA
ggggcggggtggggcggggaaatcatctcaaccactcacatccactaccaacactcthhtcaactacathhhhhbaaaaaaaaaaaaaaaaaaaaaaaaa

SEQ ID NO: 34 - DNA, 100 bp, synthetic construct; identical to SEQ ID NO: 13
  misc_feature 1..100: note = Artificial Sequence
  source 1..100: mol_type = other DNA

SEQ ID NO: 35 - DNA, 100 bp, synthetic construct
  misc_feature 1..100: note = Artificial Sequence
  source 1..100: mol_type = other DNA
ggggcggggtggggcggggaaatcatctcaaccactcacatccactaccaacactcthhtaccatacathhhhhbaaaaaaaaaaaaaaaaaaaaaaaaa

SEQ ID NO: 36 - DNA, 100 bp, synthetic construct; identical to SEQ ID NO: 12
  misc_feature 1..100: note = Artificial Sequence
  source 1..100: mol_type = other DNA

SEQ ID NO: 37 - DNA, 80 bp, synthetic construct
  misc_feature 1..80: note = Artificial Sequence
  misc_feature 39..48: note = n is a, c, g, or t
  misc_feature 80: note = n is a, c, g, or t
  source 1..80: mol_type = other DNA
ctacacgacgctcttccgatctatggtgagcaagggcgnnnnnnnnnnttttttttttttttttttttttttttttttvn

SEQ ID NO: 38 - DNA, 54 bp, synthetic construct
  misc_feature 1..54: note = Artificial Sequence
  source 1..54: mol_type = other DNA
aatgatacggcgaccaccgagatctacactctttccctacacgacgctcttccg

SEQ ID NO: 39 - DNA, 55 bp, synthetic construct
  misc_feature 1..55: note = Artificial Sequence
  source 1..55: mol_type = other DNA
caagcagaagacggcatacgagatatatcagtgtgactggagttcagacgtgtgc

SEQ ID NO: 40 - DNA, 24 bp, synthetic construct
  misc_feature 1..24: note = Artificial Sequence
  source 1..24: mol_type = other DNA
ccactcacatccactaccaacact

SEQ ID NO: 41 - DNA, 27 bp, synthetic construct
  misc_feature 1..27: note = Artificial Sequence
  source 1..27: mol_type = other DNA
gcagcgtcagatgtgtataagagacag

SEQ ID NO: 42 - DNA, 61 bp, synthetic construct
  misc_feature 1..61: note = Artificial Sequence
  source 1..61: mol_type = other DNA
aatgatacggcgaccaccgagatctacactaatcttaacactctttccctacacgacgctc

SEQ ID NO: 43 - DNA, 62 bp, synthetic construct
  misc_feature 1..62: note = Artificial Sequence
  source 1..62: mol_type = other DNA
aatgatacggcgaccaccgagatctacactcgtcggcagcgtcagatgtgtataagagacag

SEQ ID NO: 44 - DNA, 88 bp, synthetic construct
  misc_feature 1..88: note = Artificial Sequence
  source 1..88: mol_type = other DNA
caagcagaagacggcatacgagatctatcgctgtgactggagttcagacgtgtgctcttccgatcttcacatccactaccaacactct

SEQ ID NO: 45 - protein, 181 aa, synthetic construct
KPHIDNYLHDKDKDEKIEQYDKNVKEQASKDKKQQAKPQIPKDKSKVAGYIEIPDADIKEPVYPGPATREQLNRGVSFAEENESLDDQNISIAGATFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 46 - protein, 181 aa, synthetic construct
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITADDLNRETGVWETRKILVATEVK

SEQ ID NO: 47 - protein, 181 aa, synthetic construct
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETAKILVATEVK

SEQ ID NO: 48 - protein, 181 aa, synthetic construct
KPHIDNYLHDKDKDEKIEQYDKNVKEQASKDKKQQAKPQIPKDKSKVAGYIEIPDADIKEPVYPGPATREQLNRGVSFAEENESLDDQNISIAGATFIGRPNYQFTNLKAAKKGSMVYFKVGNETRKYKMTSIRNVKPTAVGVLDEQKGKDKQLTLITADDLNRETGVWETAKILVATEVK

SEQ ID NO: 49 - protein, 181 aa, synthetic construct
KPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKILVATEVK

SEQ ID NO: 50 - protein, 181 aa, synthetic construct
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVGVLDEQKGKDKQLTLITCDDYNEETGVWETRKIFVATEVK

SEQ ID NO: 51 - protein, 181 aa, synthetic construct; identical to SEQ ID NO: 27

SEQ ID NO: 52 - protein, 181 aa, synthetic construct
KPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTALKAAAKGSMVAFKVGNETRKYKMTSIRDVKPTDVGVLDEQKGKDKQLTLITCDDYNEKTGVWEKRKIFVATEVK

SEQ ID NO: 53 - protein, 169 aa, synthetic construct
SVLQAQMAAQQLPVIGGIAIPELGINLPIFKGLGNTELIYGAGTMKEEQVMGGENNYSLASHHIFGITGSSQMLFSPLERAQNGMSIYLTDKEKIYEYIIKDVFTVAPERVDVIDDTAGLKEVTLVTCTDIEATERIIVKGELKTEYDFDKAPADVLKAFNHSYNQVST

SEQ ID NO: 54 - protein, 214 aa, synthetic construct
EDKQERANYEKLQQKFQMLMSKHQEHVRPQFESLEKINKDIVGWIKLSGTSLNYPVLQGKTNHDYLNLDFEREHRRKGSIFMDFRNELKNLNHNTILYGHHVGDNTMFDVLEDYLKQSFYEKHKIIEFDNKYGKYQLQVFSAYKTTTKDNYIRTDFENDQDYQQFLDETKRKSVINSDVNVTVKDRIMTLSTCEDAYSETTKRIVVVAKIIKVS

SEQ ID NO: 55 - protein, 207 aa, synthetic construct
KTTIQKYTRNVETLEPAQAKHLKEEAALYNQYIYTKSQYQSWNKAVPEYKKQLITDKDKVIAYLSIPQIKITNIPVYSGDGEETLAAGVGHIPQTSLPIGGENTHAVLSAHSGHINNTLFSDLEDLKMKDVFYIHVLDQTLKYEIFERKIVNPEDTDAINVIPGKDLVTLVTCWPTGINNKRLLVTGRRVATTTMTPQEHIQRNKYG

SEQ ID NO: 56 - protein, 176 aa, synthetic construct
KLIDTNTKTEQTLKEAKLAAKKPQEASGTKNSTDQAKNKASFKPETGQASGILEIPKINAELPIVEGTDADDLEKGVGHYKDSYYPDENGQIVLSGHRDTVFRRTGELEKGDQLRLLLSYGEFTYEIVKTKIVDKDDTSIITLQHEKEELILTTCYPFSYVGNAPKRYIIYGKRVT

SEQ ID NO: 57 - protein, 191 aa, synthetic construct
TNVRAHAQANQAASNLQDDWANGKRSPGSFEPGQGFALLHIPKLDVVVPIAEGISSKKVLDRGMVGHYAEDGLKTAMPDAKAGNFGLAGHRNTHGEPFRYINKLEPGDPIVVETQDKYFVYKMASILPVTSPSNVSVLDPVPKQSGFKGPGRYITLTTCTPEFTSKYRMIVWGKMVEERPRSKGKPDALVS

SEQ ID NO: 58 - protein, 202 aa, synthetic construct
SLWWTNVVADRAADKQAEKVRDDWAQDRVGGSGQDGPGALDTKAGIGFLHVPAMSEGDILVEKGTSMKILNDGVAGYYTDPVKATLPTSDEKGNFSLAAHRDGHGARFHNIDKIEKGDPIVFETKDTWYVYKTYAVLPETSKYNVEVLGGIPKESGKKKAGHYITLTTCTPVYTSRYRYVVWGELVRTEKVDGDRTPPKELR

SEQ ID NO: 59 - DNA, 118 bp, synthetic construct
  source 1..118: mol_type = other DNA
gacagatagcaccaggtcagactgggaagatagcggattacaactataagttgcccgatgattttacggggtgcgtaatcgcatggaactcaaacaacctcgactccaaagtaggtgg

SEQ ID NO: 60 - DNA, 302 bp, synthetic construct
  source 1..302: mol_type = other DNA
gtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtggtaattataattacttgtatcgcctgtttcgaaagagcaatttgaagccttttgagcgggatatttcaaccgaaatttaccaagcaggcagtacgc

SEQ ID NO: 61 - DNA, 213 bp, synthetic construct
  source 1..213: mol_type = other DNA
gacagatagcaccaggtcagactgggaagatagcggattacaactataagttgcccgatgattttacggggtgcgtaatcgcatggaactcaaacaacctcgactccaaagtaggtggtaattataattacttgtatcgcctgtttcgaaagagcaatttgaagccttttgagcgggatatttcaaccgaaatttaccaagcaggcagtacgc

SEQ ID NO: 62 - DNA, 207 bp, synthetic construct
  source 1..207: mol_type = other DNA
gtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtgg

SEQ ID NO: 63 - DNA, 86 bp, synthetic construct; identical to SEQ ID NO: 24
  source 1..86: mol_type = other DNA

SEQ ID NO: 64 - protein, 147 aa, synthetic construct
QAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 65 - protein, 181 aa, synthetic construct
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTNLAAAKKGSMVYFKVGNETRKYKMTSIRNVKPTAVGVLDEQKGKDKQLTLITCDDLNRETGVWETRKILVATEVK

SEQ ID NO: 66 - protein, 181 aa, synthetic construct
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTALKAAAKGSMVAFKVGNETRKYKMTSIRNVKPTAVGVLDEQKGKDKQLTLITCDDLNRETGVWETRKILVATEVK

SEQ ID NO: 67 - protein, 170 aa, synthetic construct
AAKKGPVPAGCMKTPKPIVPVKYSIDGMKASAKVLSRGVDETGAAGAPPKNDPSSMAWFNQGPKIGSDKGNAVLTAHTYHKGGALGNRLYDKNNGIKKGDIIRLTDKTGQTVCYRYDHDTKVMVKDYNPNSNILYDNNGPAQAAIVICWDYVKKTGEFDSRVIFYTYPVA

SEQ ID NO: 68 - protein, 5 aa, synthetic construct
LPETG

SEQ ID NO: 69 - protein, 5 aa, synthetic construct
LMVGG